(19) United States
(12) Patent Application Publication
(10) Pub. No.: US 2008/0214619 A1
(43) Pub. Date: Sep. 4, 2008

(54) Business method to treat and/or prevent a gastric acid disorder with a proton pump inhibitor (PPI) and a cholinergic agonist to induce rapid onset of PPI action with or without food

(76) Inventors: M. Michael Wolfe, Newton, MA (US); Larry R. Brown, Newton, MA (US); Peter J. Manso, Parkland, FL (US)

Correspondence Address: EDWARDS ANGELL PALMER & DODGE LLP, P.O. BOX 55874, BOSTON, MA, 02205, US

(21) Appl. No.: 11/830,787

(22) Filed: Jul. 30, 2007

Related U.S. Application Data
(60) Provisional application No. 60/834,068, filed on Jul. 29, 2006.

(51) Int. Cl. A61K 31/435; A61K 31/27; A61K 31/4178; A61P 43/00

(52) U.S. Cl. 514/338; 514/478; 514/397; 514/506

(57) ABSTRACT

Pharmaceutical proton pump inhibitor (PPI) medications and methods are disclosed for preventing and/or treating gastrointestinal disorders characterized by abnormalities in gastric acid secretion at anytime of the day or night without the need for food effect or to be taken with food. The medications comprise a PPI and a cholinergic agonist for inducing rapid onset of PPI action, for increasing the duration of PPI efficacy and for optimizing clinical PPI effectiveness that may be administered at any time of the day or night without food or on an empty stomach, and possibly on an as-needed or on demand basis. In carrying out the methods, a PPI and cholinergic agonist may be administered together as a single unitary dose in the form of a liquid or solid, or administered together, but separately as either liquids or solids or a combination thereof. Preferably, an oral solid dosage form of the present invention allows for release of a proton pump inhibitor at a pH of about 5 or higher, e.g., pH about 5.5, 6, 6.5 or 7, followed by release of a cholinergic agonist within between about 10 minutes and about 60 minutes, preferably within about 15 minutes and about 30 minutes, after release of the proton pump inhibitor from the dosage form, so that it can be administered at any time of the day or night independent of food or food effect. It is believed that the methods and compositions of the present invention will increase the duration of PPI efficacy by between at least about 5 fold and about 10 fold or even about 20 fold, as compared to the duration of PPI efficacy derived from current PPI dosage forms administered alone and without food or a food effect. Kits comprising a PPI, a cholinergic agonist and optionally an antacid are disclosed, such as kits containing each drug in conventional and commercially available dry or liquid dosage forms for simultaneous or concomitant administration or in dry dosage forms to provide for the easy preparation of a liquid composition from the dry dosage forms. These new medications and methods will simplify the traditional continuous PPI regimen and improve patient compliance.

### CROSS-REFERENCE TO RELATED APPLICATIONS

The present application claims priority to U.S. Provisional Patent Application Ser. No. 60/834,068 entitled “Business method to treat and/or prevent a gastric acid disorder with a proton pump inhibitor (PPI) and a cholinergic agonist to induce rapid onset of PPI action with or without food,” filed Jul. 29, 2006.

### FIELD OF THE INVENTION

The present invention relates to the use of a proton pump inhibitor (hereinafter, a “PPI”) and a cholinergic agonist at any time of the day or night to induce rapid PPI onset of action, to increase the duration of PPI efficacy and/or to optimize clinical PPI effectiveness, without the need of food effect or to be taken with food, to treat and/or prevent gastrointestinal disorders characterized by abnormalities in gastric acid secretion. The present invention also relates to the use of a PPI and a cholinergic agonist at night, without food or food effect, to treat and/or prevent nocturnal heartburn and nocturnal acid breakthrough.

### BACKGROUND OF THE INVENTION

Today, millions of people suffer from a wide variety of gastrointestinal disorders involving gastric acid secretion. Such disorders or conditions include, but are not limited to, gastroesophageal reflux disease (GERD), poorly responsive symptomatic GERD, peptic ulcer disease, duodenal ulcers, gastric ulcers, atrophic gastritis, esophagitis, severe erosive esophagitis, mild distal esophagitis, heartburn, nocturnal heartburn, episodic heartburn, acid break-through, pathological gastrointestinal hypersecretory disease such as Zollinger-Ellison syndrome(ZES), no nucler dyspepsia, sour stomach, abdominal pain, abdominal discomfort, gastroparesis, acid-related asthma, cough or apnea, gastroesophageal reflux with pyrosis and the like. These conditions are thought to be caused by an imbalance between aggressive factors, namely acid and pepsin production, and defensive factors referred to as mucus, bicarbonate and prostaglandin production. These above-listed conditions commonly arise in both healthy and critically ill patients, and may be accompanied by significant complications, such as upper gastrointestinal bleeding and other symptoms.

While conditions such as ZES and peptic ulcers, in particular, can have serious complications and represent some of the most prevalent diseases in industrialized nations, GERD is by far the most common disorder seen by gastroenterologists and primary care physicians. The wide diversity of symptoms and disease severity produced by acid reflux has led to the need for more individualized and effective treatment strategies.

Various methods and agents have been used to treat and/or attempt to eradicate these gastrointestinal disorders. They include special diets, refraining from ingestion of certain foods, exercise, meditation, and the administration of various pharmaceutical agents such as antacids, histamine H₂-receptor antagonists, PPIs and antimicrobials.

Therapeutic agents believed to be effective in the treatment of GERD include gastric acid suppressing agents, such as histamine H₂-receptor antagonists and proton pump inhibitors. In addition, other that may be used agents include antacids/alginates, sucralfates and prokinetic agents. These agents can be distinguished by their mechanisms of action, safety profile, pharmacokinetics and indications. Antacids and alginates are still widely used. Even though antacids and alginates have short durations of action, they are inexpensive, easy to use and safe. Unfortunately, antacids and alginates do not provide long-term symptom resolution of GERD.

In the past, histamine H₂-receptor antagonists have been the drugs of choice to treat such conditions, especially GERD. Their higher costs, as compared to antacids, are tolerated because of the clinical results obtained both in terms of symptom relief and healing. These advantages are believed to be attributable to their mode of action, which provide more potent and longer duration of effect on gastric acidity. Although histamine H₂-receptor antagonists have been used successfully in the past, the erratic and diminishing responses at times with these antagonists, as well as the progressive occurrence of side effects associated with the use of larger doses in some cases, has led to the widespread use of a class of antisecretory compounds called proton pump inhibitors (“PPIs”) in humans to treat gastric acid related diseases. Proton pump inhibitors have and continue to take market share from histamine H₂-receptor antagonists, particularly in the treatment of GERD. Proton pump inhibitors are known to offer certain advantages over histamine H₂-receptor antagonists in terms of symptom resolution, healing and prevention of relapse for reflux esophagitis.

Patients with severe symptoms, severe mucosal damage or both are generally treated with proton pump inhibitors for profound and long-term control of gastric acid secretion. Patients with mild symptoms and limited mucosal damage are believed to respond best to antacids, histamine H₂-receptor antagonists, prokinetic agents or proton pump inhibitors.

While different doses of PPIs are recommended, it is believed that PPIs are generally similar at equivalent doses when used in the treatment of acid-related disorders, including GERD. However, because PPIs are most effective when the parietal cells are stimulated to secrete acid, such as in response to a meal, patients are instructed to take PPIs daily and then only before or with the first meal of the day for optimal efficacy.

Thus, currently, there is a proper way and sub-optimal way to use PPIs. It is believed that the proper way to use PPIs is to instruct patients to take PPIs 15 to 30 minutes before their first meal, preferably before breakfast and, for those patients who do not eat breakfast, they should be instructed to take PPIs 15 to 30 minutes before lunch. This is because it takes about 15 minutes for the PPIs to be absorbed into patients' systems, and it is necessary to wake up the acid-secreting pumps with food, the time at which the greatest recruitment of the H⁺, K⁺ATPase occurs. However, and quite often, when PPIs are prescribed, physicians make the mistake of not giving patients enough instructions on how and when to properly take PPIs. In addition, the physicians often fail to emphasize to patients just how important it is to take PPIs before the breakfast meal to stimulate acid secretion. Unfortunately, if these instructions are not given or followed and a patient's stomach remains empty after taking a PPI, the duration of PPI efficacy can be diminished by 5 to 20 fold and clinical effectiveness can be seriously compromised, in many cases by more than 50%. See Barrison A F, Jarboe L A, Weinberg B M, Nimmagadda K, Sullivan L M, Wolfe M M.: Use patterns of proton pump inhibitors in clinical practice. Am J Med, 111:469-73 (2001), which is incorporated herein by reference in its entirety.

Patients are also instructed that PPIs should not be used concomitantly with H₂-receptor antagonists, prostaglandins, or other antisecretory agents, such as somatostatin and its analogs, and drugs that possess anticholinergic properties, because it is believed that such concomitant use will markedly reduce the acid inhibitory effects of the PPIs.

Proton pump inhibitors are drugs which suppress gastric acid secretion by specific irreversible inhibition of the H+,K+ adenosinetriphosphatase (H+,K+-ATPase) enzyme (proton pump) at, in or near the secretory surface of the gastric parietal cells. The H+,K+-ATPase enzyme is involved in the final step of hydrogen ion secretion by the parietal cells. The gastric enzyme H+,K+-ATPase is a member of the family of ATPase enzymes. It is believed that the H+,K+ ATPase is co-expressed tightly bound to a β-subunit that is smaller than its catalytic or α-subunit and consists of eight transmembrane segments for the catalytic α-subunit and one transmembrane segment for the β-subunit.

All PPIs share a common structural motif, a substituted pyridylmethylsulfinyl benzimidazole, as well as common inhibitory mechanisms. In addition, all PPIs are weak protonatable pyridines. Once PPIs accumulate selectively in the acid spaces of the secreting parietal cells, they undergo an acid catalyzed conversion within those spaces or on the surface of the enzymes to a reactive species, the thiophilic sulfenamide or sulfenic acid. The reactive species to which the PPIs have been converted are permanent cations. The rate of conversion varies among the PPIs and is a function of their pKa and other structural features. Once the PPIs are converted to the permanent cations, these cations react with cysteine residues available from the external surfaces of the gastric enzyme that faces the lumen of the secretory space of the parietal cells to form disulfide bonds. It is believed that this covalent reaction between the gastric enzyme and the permanent cations that is responsible for the inhibition of the gastric enzyme.

Omeprazole and lansoprazole are examples of proton pump inhibitors. Omeprazole is a substituted benzimidazole, 5-methoxy-2-[(4-methoxy—3,5-dimethyl-2-pyridinyl)methyl]sulfinyl]-1H-benzimidazole. Omeprazole, like other proton pump inhibitors, does not exhibit anti-cholinergic or histamine H₂— receptor antagonist properties. Drugs of this class suppress gastric acid secretion by the specific inhibition of the H⁺,K⁺ ATPase enzyme system (proton pump) at, in or near the secretory surface of the gastric parietal cells.

Generally speaking, omeprazole, lansoprazole, rabeprazole, pantoprazole, esomeprazole and other proton pump inhibitors are formulated as (a) an enteric-coated solid dosage form, either a delayed-, modified- or sustained-release capsule or tablet, (b) an intravenous solution, or (c) a product for reconstitution, and are prescribed for daily, continuous treatment of, for example, active duodenal ulcers, gastric ulcers, gastroesophageal reflux disease (GERD), severe erosive esophagitis, poorly responsive symptomatic GERD, and pathological hypersecretory conditions such as Zollinger Ellison syndrome.

Even though PPIs are without question the most potent inhibitors of gastric acid secretion available today when directions for administration are strictly followed, PPIs are not without drawback. In one disadvantage, PPI onset of action, i.e., inhibition of H⁺,K⁺ ATPase, at the start of PPI therapy is generally delayed from about 24 to about 96 hours following oral consumption. This delay in onset of action results because PPIs generally require accumulation and acid activation in the parietal cells before onset of action can occur.

In another disadvantage, once PPI therapy has been initiated, PPIs should be administered daily with food to initiate parietal cell activation and acid secretion to maximize onset an duration of action and clinical efficacy. As indicated, PPIs require acid secretion stimulation for maximum efficacy and therefore require strict administration compliance for maximum clinical effectiveness. Consequently, it is recommended that PPIs be administered with a meal and, preferably, before the first meal of the day, to initiate sufficient acid secretion. When taken optimally before breakfast, PPI inhibition of acid secretion is significantly inhibited; however, it becomes clinically significant only after several hours. Also, once-daily PPI dosing results in about 66% steady-state inhibition of necessarily occur immediately and can be delayed in some patients for quite some time due to the slow onset of action associated with PPI therapy.

In yet another disadvantage, because of their unique pharmacokinetic properties, the occasional use of a PPI taken on an “as needed” basis or the use at times of a day other than before or with meals (no food effect) may not provide adequate acid inhibition or produce a consistent or satisfactory clinical response. In contrast to antacids or H₂-receptor antagonists, PPIs are limited to chronic daily use at meal time, preferably the first meal of the day time, to stimulate and maximize onset of action. Consequently, there is less flexibility or convenience with PPI administration and therefore PPIs are not necessarily suitable for “on demand” or “as needed” (prn) use to treat excess gastric acid secretion disorders, especially when compared to antacids or H₂-receptor antagonists, both of which can be taken on demand or as needed and used at any time during the day or night.

In a further disadvantage, it is well known that proton pump inhibitors are susceptible to degradation/activation in acid reacting and neutral media. With respect to the stability properties, acid susceptible proton pump inhibitors should be protected from contact with acidic gastric juice by an enteric coating layer or other means when the PPI is delivered orally. There are different enteric coating layered preparations of proton pump inhibitors described in the literature. See, for example, U.S. Pat. No. 4,786,505, which describes an enteric-coated preparation comprising omeprazole, which patent is incorporated herein by reference in its entirety.

There are also problems with producing a fixed unit dosage form comprising a rather high amount of active substance. Different active substances with differing physical properties in the same preparation give further problems. Preparation of a multiple unit tableted dosage form encounters specific problems when enteric coating layered pellets or granules containing acid susceptible proton pump inhibitors are compressed into tablets or other oral solid dosage forms. If the enteric coating layer does not withstand the compression of the pellets or granules into a tablet, the susceptible PPI will be activated prematurely by penetrating acidic gastric juices, i.e., the PPI will undergo acid catalyzed conversion within the stomach to the reactive species, the thiophilic sulfenamide or sulfenic acid, which will not be absorbed and will thus not inhibit acid secretion.

Despite their widespread use, there is a need to improve PPI clinical efficacy and duration of action, especially without food effect, and the speed at which PPI onset of action occurs, so that PPIs can be used effectively at any time of the day or night, even possibly on demand or an “as needed” or prn basis, to treat gastrointestinal disorders characterized by excess gastric acid secretion, namely, ZES, peptic ulcer disease, duodenal ulcers, atrophic gastritis, GERD (esophagitis, heartburn, nocturnal heartburn, episodic heartburn, regurgitation and other manifestations of GERD), abdominal or esophageal pain and dyspepsia.

### SUMMARY OF THE INVENTION

In brief, the present invention alleviates and overcomes certain of the above-identified problems and shortcomings of the present state of proton pump inhibitor therapy through the discovery of novel combination pharmaceutical compositions comprised of proton pump inhibitor and a cholinergic agonist for oral administration, novel methods of treatment and/or prevention of gastrointestinal disorders with such pharmaceutical compositions and novel instruction.

In accordance with the present invention, a combination pharmaceutical composition for administration is provided to induce rapid PPI onset of action, without food effect, to treat and/or prevent gastrointestinal disorders in individuals at any time of the day without a food effect requirement. Quite remarkably and surprisingly, the present invention eliminates the food effect requirement currently associated with PPI therapy and now makes it possible to use PPI therapy conveniently to effectively treat or prevent gastrointestinal disorders at any time of the day or night, even when treatment is initiated in a fasted state and without food. Thus, it is now thought possible to take PPI therapy during the evening or night or at bedtime without food and on an empty stomach, rather than in the morning before or with breakfast as currently instructed, to effectively treat and/or prevent the symptoms associated with gastrointestinal disorders, especially nocturnal heart burn and nocturnal acid breakthrough commonly experienced by individuals undergoing PPI therapy. This is believed to be a significant improvement overcoming the disadvantages of current PPI therapy, i.e., the requirement of food activation and PPI use with the first meal of the day thereby preventing PPI use during the evening or night or at bedtime.

Generally speaking, the combination pharmaceutical compositions of the present invention comprise a proton pump inhibitor and a cholinergic agonist. The combination pharmaceutical compositions are administered in amounts that are safe and effective to induce rapid PPI onset of action within between about 15 minutes to about 120 minutes or less, preferably within between about 15 minutes and 45 minutes, without food effect, following administration. Quite remarkably, this is accomplished through the discovery that, when a PPI is co-administered with a cholinergic agonist in an effective amount, sufficient parietal cell recruitment and activation and gastric acid production can be generated by the cholinergic agonist within a short period of time to induce rapid PPI onset of action following PPI absorption, without the need to take the PPI before or with meals or on a full stomach. Even more remarkable, it is believed that the use of a cholinergic agonist in combination with a PPI can effectively induce PPI onset of action within about 15 minutes to about 120 minutes or more, preferably within about 15 minutes to about 45 minutes, following oral administration, even when PPI therapy is first initiated. As a result, it is now believed to be possible to initiate and use PPI therapy at any time of the day or night, even possibly “on demand” or “as needed”, without the need to take the PPI before or with meals, to treat a wide variety of gastrointestinal disorders and symptoms associated therewith, including heartburn, whether the heartburn is acute, chronic, episodic or nocturnal or due to acid-breakthrough. Moreover, the present invention eliminates the food effect requirement to maximize PPI clinical effectiveness and duration of action and now allows individuals to take PPIs in a more convenient and less rigid manner. Thus, it is believed that the present invention will simplify traditional PPI administration regimens and provide flexible administration, improve patient compliance, enhance the speed at which PPI onset of action and relief occurs, maximize PPI clinical effectiveness and increase duration of action, regardless of what time of the day or night the pharmaceutical compositions of the present invention are used and whether or not the pharmaceutical compositions of the present invention are taken on a full or empty stomach, especially as compared to when PPIs are taken alone and without food or in a fasted state. In other words, while the pharmaceutical compositions of the present invention may be taken with food, the present invention affords use, unlike current PPI therapy, at any time, day or night, without food to treat or prevent gastrointestinal disorders.

In accordance with the present invention, the combination pharmaceutical compositions are administered in safe and effective amounts at least once or twice a day preferably as an oral dose, at any time of the day or night, when convenient. Preferably, the PPI and the cholinergic agonist are administered simultaneously as individual separate doses or as a single unitary combination dose in an oral dosage form. Nevertheless, the PPI and the cholinergic agonist may be co-administered at separate times of one other in individual dosage forms. When concomitantly administered, in contrast to simultaneous co-administration, it is preferred to first take the PPI first followed by the cholinergic agonist and that the PPI and the cholinergic agonist should be administered within at least about two hours or less of one another and preferably within at least about 60 minutes of one another and more preferably within at least about 30 minutes of one another and more preferably within at least about 5 minutes to about 15 minutes of one another and most preferably, they should be taken simultaneous or one right after the other. While the PPI or the cholinergic agonist may be taken or administered first in accordance with the present invention, it is preferable as indicated above, but not essential, to take or administer the PPI first or in advance of the cholinergic agonist whenever they are co-administered as individual or separate doses. Nevertheless, to maximize convenience and improve compliance, it is most preferable to take or administer each drug simultaneously or at least within about 5 to about 15 minutes or less of one another and more preferably within about 1 to 60 seconds of each other when each drug is co-administered as individual or separate doses. It should be understood by those versed in this art that, while oral administration is the preferred route of administration, the pharmaceutical combinations, including the combination compositions, of the present invention may be administered in any suitable and effective dosage form, including injection, inhalation, intranasal, transdermal, topical and rectal dosage forms. Nonetheless, it should be understood that when the PPI and the cholinergic agonist are orally co-administered at the same or different times, regardless of which drug is administered first, it is preferred for the PPI to be released from the oral dosage form at a pH of about 5 or greater in the gastroinstestinal tract and for the cholinergic agonist to be released from the same or a different oral dosage form within between about 10 minutes and 60 minutes after release of the PPI, and more preferably within between about 15 minutes and 45 minutes after release of the PPI, and even more preferably within between about 20 minutes and 40 minutes after release of the PPI, and most preferably within about 30 minutes after release of the PPI.

It also is found that, when a pharmaceutical combination of the present invention is administered at least once or twice a day as an oral dose, the oral PPI dose provides a much faster onset of action in acid reduction effect over that which is obtained when using the PPI alone and without food or on an empty stomach. In fact, it is believed that the pharmaceutical compositions of the present invention, when administered as an oral dose, will reduce acid output levels to basal acid output levels within about 45 minutes to about 120 minutes or less, without a food effect, following consumption, as compared to the much slower onset of action when the PPI is administered alone on an empty stomach. It is also believed that, when the pharmaceutical compositions of the present invention are administered at least once or twice a day as an oral dose, the oral dose (1) is at least as effective as, if not more effective than, when the PPI is administered alone, (2) does not require food effect for onset of action, and (3) induces rapid onset of action even when administered (a) daily without food or (b) at the start of PPI therapy, as compared to the delay or slower speed at which onset of action occurs when a PPI is administered or used alone without food or on an empty stomach.

Quite surprisingly, it is believed that the pharmaceutical combinations of the present invention may possibly be used periodically (other than daily) and at anytime of the day or night “on demand” or “as needed” without food, much like the H₂-receptor antagonists, or in step-down therapy, when treating and/or preventing certain gastrointestinal disorders, such as GERD, episodic or nocturnal heartburn, nocturnal acid breakthrough, sour stomach, upset stomach and the like. Of course, it should be understood by those versed in this art, that the pharmaceutical compositions of the present invention are best suited for use on traditional continuous daily regimens, like those regimens currently prescribed for PPIs, but with or without food or on an empty stomach or in a fasted state, to treat gastrointestinal issues. Thus, in accordance with the present invention, PPI therapy can be administered either possibly on demand or as needed or as a traditional regimen without food, while achieving rapid onset of action and relief without a food effect requirement.

While the pharmaceutical combinations of the present invention contemplate the combination of (a) proton pump inhibitor and (b) a cholinergic agonist in any form, the use of the pharmaceutical combinations in accordance with the present invention will depend upon the form of delivery chosen. For example, preferred pharmaceutical compositions for oral administration when swallowed are those comprised of a proton pump inhibitor in a modified release or enteric coated form, and a cholinergic agonist in an immediate or modified release form to permit release of the PPI in the small intestine to avoid premature luminal acid activation followed by release of the cholinergic agonist. On the other hand, preferred compositions for oral buccal administration are those comprised of a proton pump inhibitor and a cholinergic agonist in which (a) both drugs are in immediate release form to permit early or rapid release of the PPI for absorption in the oral cavity release of the cholinergic agonist in the gastrointestinal tract, or (b) the proton pump inhibitor is in a modified release or enteric coated form layered around the cholinergic agonist is in an immediate or modified release form to permit initial release of the PPI in the small intestine at a pH of about 5 or greater followed by release of the cholinergic agonist once swallowed. Alternatively, the pharmaceutical compositions for oral administration (swallowing) may contain the PPI and the cholinergic agonist in immediate release forms, so long as the PPI following entry into the stomach can be effectively absorbed from the small intestine, without undergoing premature luminal acid activation, and the cholinergic agonist can be absorbed after the PPI is effectively absorbed, to accomplish the objectives of the present invention.

In carrying out the present invention, the pharmaceutical compositions can be administered to humans and other animal species, such as bovines, canines, felines, porcines, equines, sheep, rabbits, mice, rats, rodents, monkeys, etc. Thus, the terms “patient(s)”, “subject(s)”, and “individual(s)” are used herein interchangeably and in a broad sense and each is meant to include all appropriate animal species. The inventive pharmaceutical compositions can be formulated as powders, tablets, suspension tablets, chewable tablets, rapid melt/quick dissolve tablets, troches, capsules, tablets inside of capsules, caplets, caplets inside of capsules, effervescent powders, effervescent tablets, pellets, granules and liquids/solutions/suspensions by way of example.

Optionally, and in accordance with the present invention, other excipients or agents, such as binders, fillers, lubricants, swellers, glidants, disintegrants, taste masking agents, diluents, neutralizing and buffering agents, permeabilizers, sweeteners, flavors, anti-flatulents, parietal cell activators, etc. may be included in the pharmaceutical compositions or administered concomitantly with the pharmaceutical compositions in appropriate regimens which complement the beneficial effects of the pharmaceutical compositions of the present invention, so long as such additives do not defeat the objectives of the present invention.

Kits are also disclosed herein to provide a PPI and a cholinergic agonist in separate oral dosage forms for concomitant administration or to utilize dry dosage forms to provide for the easy preparation of a liquid composition from the dry forms.

In accordance with the present invention, there is further provided a method of treating and/or preventing a gastric acid disorder in a patient in need of such therapy by administering to a patient a pharmaceutical composition comprising a proton pump inhibitor in a pharmaceutically acceptable carrier and at least one cholinergic agonist, wherein the administering step comprises providing a patient with a single unitary combination dose of the composition without requiring separate administration of the cholinergic agonist.

In accordance with the present invention, there is further provided a method of treating and/or preventing a gastric acid disorder in a patient in need of such treatment by administering to a patient a pharmaceutical composition comprising a proton pump inhibitor and at least one cholinergic agonist, wherein the administering step comprises providing a patient with an individual PPI dose and an individual cholinergic agonist dose, and wherein both doses may be taken together or simultaneously by the patient or each dose may be consumed separately in either order by the patient. In addition, the present invention contemplates instructing a patient in need of gastric acid disorder therapy to take an individual PPI dose and an individual cholinergic agonist dose together or simultaneously or concomitantly or separately in either order, preferably the PPI first, to treat or prevent a gastric acid disorder.

Also in accordance with the present invention, there is provided a method of treating and/or preventing a gastric acid disorder in a patient in need of treatment by administering to a patient a pharmaceutical composition, including a combination composition, comprising a proton pump inhibitor and at least one cholinergic agonist and, optionally, an antacid, each in a pharmaceutically acceptable carrier wherein the pharmaceutical composition or combination can be administered at any time of the day or night as a traditional continuous, daily regimen, without food or on an empty stomach, while achieving rapid onset of action and effective relief. It should be understood that the pharmaceutical compositions and combinations may be administered “on demand” or “as needed” to possibly achieve rapid onset of action and effective relief, as compared to when a proton pump inhibitor is administered alone without food or on an empty stomach. The present invention also contemplates instructing a patient in need of gastric acid disorder therapy to take a pharmaceutical composition comprising a proton pump inhibitor and a cholinergic agonist and, optionally, an antacid, at any time of the day or night possibly “on demand” or “as needed” or as a traditional continuous, daily regimen, with or without food or on an empty stomach, to treat or prevent a gastric acid disorder. It is believed that, when a PPI and a cholinergic agonist are administered with an antacid in accordance with this present invention, the PPI may be effective on demand relative to when a proton pump inhibitor is administered alone without food or on an empty stomach.

The present invention also contemplates methods of doing business to treat or prevent a gastric acid disorder in an individual with a proton pump inhibitor, a cholinergic agonist and, optionally, an antacid, which include manufacture, distribution, repackaging, dispensing and/or instruction of these active ingredients individually or as unitary and single pharmaceutical compositions, in numerous dosage forms and strengths, so that once the proton pump inhibitor, the cholinergic agonist and, optionally, the antacid have been dispensed by a retailer, such as a pharmacy, to an individual, the individual may take the proton pump inhibitor, the cholinergic agonist and, optionally, an antacid at any time of the day or night as a strict daily regimen, with or without food or a meal or on an empty stomach, to treat or prevent the gastric acid disorder.

Thus, it is believed that the present invention now makes possible to treat and/or prevent acute or periodic gastric acid disorders, such as episodic or nocturnal heartburn or nocturnal acid break through, effectively and quickly with PPIs, even possibly on an “on demand” or “as needed” basis or during step-down PPI therapy.

Additionally, the present invention relates to a method for further enhancing the pharmacological activity of an administered proton pump inhibitor in which at least one parietal cell activator, other than the cholinergic agonist, is orally administered to the patient before, during and/or after the oral administration of the proton pump inhibitor and the cholinergic agonist.

The present invention also relates to a method of using a proton pump inhibitor in the treatment of a patient suffering from a gastric acid disorder that comprises providing the patient with an effective gastric acid suppressing amount of a proton pump inhibitor and an effective parietal cell activation amount of a cholinergic agonist and informing the patient that the administration of a proton pump inhibitor with the cholinergic agonist results in an increase in at least one of C(max) and AUC(last) of the proton pump inhibitor at the time of parietal cell activation as compared to administration of the proton pump inhibitor without food.

The present invention also relates to a method of improving the duration of action of a proton pump inhibitor by, for example, up to about 5 fold to about 10 fold or even 20 fold without food effect in a patient receiving proton pump inhibitor therapy comprising administering to the patient at any time of the day or night independent of food, an effective gastric acid suppressing amount of a proton pump inhibitor and an effective parietal cell activation amount cholinergic agonist each in the same or separate pharmaceutical compositions.

The present invention also relates to a method of improving bioavailability of a proton pump inhibitor at the time of parietal cell activation in a patient receiving proton pump inhibitor therapy comprising administering to the patient an effective gastric acid suppressing amount of a proton pump inhibitor and an effective parietal cell activation amount cholinergic agonist each in the same or separate pharmaceutical compositions without food or the patient being in a fasted or unfed condition.

In addition, the present invention contemplates a method of using a proton pump inhibitor in the treatment of a gastric acid disorder which comprises altering the oral bioavailability of a proton pump inhibitor at the time of parietal cell activation without food effect by obtaining a proton pump inhibitor from a container providing information that administration of the proton pump inhibitor with a cholinergic agonist increases at least one of C(max) and AUC(last) of the proton pump inhibitor at the time of parietal cell activation, as compared to administration of the proton pump inhibitor without food or when the patient is in a fasted state.

In addition, the present invention contemplates a method of using a proton pump inhibitor in the treatment of a gastric acid disorder which comprises altering the oral bioavailability of a proton pump inhibitor at the time of parietal cell activation without food effect by obtaining a proton pump inhibitor from a container providing information that administration of the proton pump inhibitor with a cholinergic agonist increases the duration efficacy of the proton pump inhibitor by, for example, up to between about 5 fold and about 10 fold or even about 20 fold at the time of parietal cell activation, as compared to administration of the proton pump inhibitor without food or when the patient is in a fasted state.

The present invention further relates to a method of using a proton pump inhibitor in the treatment of a gastric acid disorder which comprises administering to a patient in need of treatment an effective gastric acid suppressing amount of a proton pump inhibitor with an effective parietal cell activation amount of a cholinergic agonist, wherein the administration of the proton pump inhibitor with the cholinergic agonist results in an increase in duration of PPI efficacy and at least one of C(max) and AUC(last) of the proton pump inhibitor at the time of parietal cell activation without food effect, as compared to administration of a proton pump inhibitor alone and without food or in a fasted state and informing the patient that the administration of the proton pump inhibitor with the cholinergic agonist results in an increase in the duration of PPI efficacy and at least one of C(max) and AUC(last) of the proton pump inhibitor at the time of parietal cell activation without food effect, as compared to administration of the proton pump inhibitor alone and without food or when the patient is in a fasted state.

The present invention further relates to method of increasing the duration of efficacy of a PPI following oral administration without food effect in a patient in need of gastric acid disorder treatment which comprises orally ingesting an effective gastric acid suppressing amount of a proton pump inhibitor in a pharmaceutical composition and mimicking a meal effect on parietal cells in the patient induced orally without food to activate the parietal cells within between about 10 minutes and about 60 minutes following release of the proton pump inhibitor from the pharmaceutical composition, so that the duration of efficacy of the proton pump inhibitor is increased by at least between about 5 and about 20 fold, as compared to the duration of efficacy of the proton pump inhibitor when orally ingested by the patient without food effect or when in a fasted state.

The present invention further relates to a method of increasing the duration of efficacy of a proton pump inhibitor following oral administration without food effect in a patient in need of treatment which comprises orally ingesting an effective gastric acid suppressing amount of a proton pump inhibitor in a pharmaceutical composition and orally inducing parietal cell activation without food in the patient within between about 10 minutes and about 60 minutes following release of the proton pump inhibitor at a pH of at least about 5 from the pharmaceutical composition, so that the duration of efficacy of the proton pump inhibitor is increased by at least between about 5 and about 20 fold, as compared to the duration of efficacy of the proton pump inhibitor when orally ingested by the patient without meal effect or when in a fasted state.

The present invention further relates to a method of optimizing clinical effectiveness of a proton pump inhibitor following oral administration in a patient in need of gastric acid disorder treatment which comprises orally ingesting an effective gastric acid suppressing amount of a proton pump inhibitor to the patient at any time of the day or night without food or when the patient is in a fasted state and mimicking food effect on parietal cells in the patient induced orally without food to activate parietal cells in the patient at a pharmacological time that will optimize clinical effectiveness of the proton pump inhibitor, so that the clinical effectiveness of the proton pump inhibitor, when ingested by the patient, is optimized in the patient, as compared to sub-optimal effectiveness of the proton pump inhibitor when ingested by the patient without food effect or when the patient is in a fasted state.

The present invention also relates to a method of increasing the duration of action and optimizing clinical effectiveness of a proton pump inhibitor following oral administration in a patient in need of gastric acid disorder treatment which comprises orally administering to the patient an effective gastric acid suppressing amount of a proton pump inhibitor at any time of the day or night without food or when the patient is in an unfed condition and orally inducing parietal cell activation without food in the patient at a pharmacological time that will increase the duration of action and optimize clinical effectiveness of the proton pump inhibitor, so that the duration of efficacy and clinical effectiveness of the proton pump inhibitor, when orally administered to the patient, are increased and optimized, as compared to reduced duration of action and sub-optimal effectiveness of the proton pump inhibitor when the PPI is administered to the patient without food effect or when the patient is in an unfed condition.

It is therefore an object of the present invention to provide a pharmaceutical composition for oral administration comprising (a) a proton pump inhibitor, and (b) a cholinergic agonist, in any suitable form for oral administration, without a food effect requirement, to effectively suppress or reduce gastric acid secretion to treat subjects, e.g., subjects diagnosed with, suffering from or predisposed to gastrointestinal disorders. While it is believed that the novel pharmaceutical compositions and methods of the present invention may be administered “on demand” or “as needed” without food to treat or prevent a gastric acid disorder, such as episodic or nocturnal heartburn, it is believed that the novel pharmaceutical compositions and methods are best suited for traditional continuous or daily PPI therapy to treat or prevent gastric acid disorders, but with unique added convenience while maximizing PPI efficacy. Quite remarkably, because the present invention uniquely mimics the effects of a meal, that is, it wakes up the acid secreting pumps without food, it is now possible for patients in need of PPI therapy to take PPIs daily at any time of the day or night without current food limitation regarding administration to effectively treat or prevent a gastric acid disorder. Thus, it is believed that the novel pharmaceutical compositions and methods of the present invention have now overcome one of the major drawbacks or limitations currently associated with traditional continuous PPI therapy, i.e., the food effect requirement and administration of PPIs 15 to 30 minutes before the first meal of the day, and now makes PPI administration convenient and simple for all patients.

Thus, and quite remarkably, it is believed that the novel pharmaceutical compositions and methods of the present invention have eliminated the food effect requirement currently required with PPI therapy by uniquely mimicking the effects of a meal as when PPIs are taken in conjunction with a meal

It is another object of the present invention to provide a pharmaceutical composition for oral administration comprising a proton pump inhibitor and a cholinergic agonist, which provides for the release of the PPI, preferably at a pH of about 5 or higher, e.g., a pH of about 6 or 7, for PPI absorption in the duodenum to avoid premature PPI luminal acid activation, and the release of the cholinergic agonist, preferably within between about 10 minutes and 60 minutes after PPI release, for absorption of the cholinergic agonist in the small intestine at the same or distal to the site of PPI absorption.

Another object of the present invention is to provide a novel dosage for, such as a multiparticulate tablet, capsule or caplet or other oral dosage form, e.g., a chewable, orally disintegratable, sublingual or buccal tablet, containing a proton pump inhibitor and a cholinergic agonist and, optionally, an antacid, that disintegrates following oral administration and provides for effective absorption of both the PPI and the cholinergic agonist.

Another object of the present invention is to ensure the stability of the enteric coating film within the oral disintegratable tablet containing a cholinergic agonist together with enteric coated proton pump inhibitor microgranules during storage.

It is also an object of the present invention to ensure integrity of the enteric film coating of the proton pump inhibitor or individual proton pump inhibitor particles or microgranules during use.

It is another object of the present invention to provide a dosage form having at least one layer, wherein a proton pump inhibitor that has been enterically coated and a cholinergic agonist have been combined in the same layer to provide release of the PPI, preferably at a pH of about 5 or higher, e.g., pH of about 6 or 7, for PPI absorption in the duodenum and release of the cholinergic agonist, preferably within between about 10 minutes and 60 minutes after PPI release for absorption of the cholinergic agonist in the small intestine distal to the site of PPI absorption.

Another object of the present invention is to provide a dosage form combining a proton pump inhibitor that has been enterically coated in one distinct layer, with a cholinergic agonist in a second separate and distinct layer, which provides preferably release of the PPI at a pH of about 5 or higher, e.g., pH of about 6 or 7, for PPI absorption in the duodenum followed by release of the cholinergic agonist within between about 10 minutes and 60 minutes after PPI release for absorption in the small intestine distal to the site of PPI absorption and which prevents premature PPI luminal acid activation.

It is another object of the present invention to provide a novel dosage form, such as a multiparticulate tablet or oral dosage form, containing a proton pump inhibitor and a cholinergic agonist, which disintegrates in the mouth and provides a good mouth feeling and taste.

Another object of the present invention is to provide a dosage form comprising a proton pump inhibitor and a cholinergic agonist either in one layer or in separate and distinct layers that is easy to swallow.

In yet another object of the present invention is to provide a novel dosage form, such as a multiparticulate tablet or caplet or oral dosage form, comprising a proton pump inhibitor and a cholinergic agonist wherein (a) the dosage form comprises at least one layer or filling and the proton pump inhibitor and the cholinergic agonist are in the same layer or filling, (b) the dosage form comprises at least two layers or fillings and the proton pump inhibitor and the cholinergic agonist are each in distinct layers or fillings.

In yet another object of the present invention is to provide a novel dosage form, such as a capsule comprising proton pump inhibitor particles or beads and cholinergic agonist particles or beads, which preferably provides for the release of the proton pump inhibitor in the small intestine at a pH of about 5 or higher followed by release of the cholinergic agonist within between about 10 minutes and 60 minutes, preferably between about 15 minutes and about 45 minutes, even more preferably within between about 20 minutes and about 40 minutes, and most preferably within about 30 minutes, after proton pump release for absorption of each drug in the small intestine and prevents premature luminal acid activation.

In yet another object of the present invention is to provide a novel single or multi-layer dosage form comprising a proton pump inhibitor, a cholinergic agonist and an antacid, e.g., a calcium, magnesium and/or aluminum antacid, in a dosage form comprising at least one, two or three layers, wherein the proton pump inhibitor, cholinergic agonist and antacid are in a single layer or the antacid and cholinergic agonist are in one layer and the proton pump inhibitor is in a second layer or various combinations thereof to form two layers or each active ingredient is in a distinct layer when a three layer dosage form is provided.

It is an object of the present invention to provide a multi-layer dosage form combining a proton pump inhibitor, free of enteric coating, in one distinct layer, with a cholinergic agonist and an antacid either together in a second distinct layer or in second and third distinct layers, respectively, which provides antacid in sufficient amount to neutralize the gastric environment so as not to cause degradation or premature luminal acid activation of the non-enterically coated proton pump inhibitor.

It is another object of the present invention to provide a dosage form combining a proton pump inhibitor free of enteric coating, in one distinct layer, with a cholinergic agonist and an antacid either together in a second distinct layer or in second and third distinct layers, respectively, which provides immediate release of the proton pump inhibitor followed by release of the cholinergic agonist.

It is another object of the present invention to provide a dosage form comprising a proton pump inhibitor and a cholinergic agonist and an antacid either in one layer, in two layers wherein two drugs are combined together in one of the two layers, or in separate and distinct layers that is easy to swallow after chewing.

It is another object of the present invention to provide a dosage form combining a proton pump inhibitor free of enteric coating and a cholinergic agonist in one distinct layer, with an aluminum, magnesium or calcium salt of an antacid in a second separate and distinct layer, that is chewable.

It is another object of the present invention to provide a dosage form combining a proton pump inhibitor free of enteric coating, in one distinct layer and a cholinergic agonist, with an aluminum, magnesium or calcium salt of an antacid in a second separate and distinct layer, or all actives in a single layer, that rapidly dissolves in the oral cavity or mouth.

It is another object of the present invention to provide a dosage form combining a proton pump inhibitor free of enteric coating in one distinct layer and a cholinergic agonist, with an aluminum, magnesium or calcium salt of an antacid in a second separate and distinct layer, or all actives in a single layer, that has taste masking ingredients to provide a chewable or rapidly dissolving dosage form substantially free of bitterness.

It is still another object of the present invention to provide a dosage form combining a proton pump inhibitor with a cholinergic agonist in which there is an effective gastric acid suppressing amount of a proton pump inhibitor to treat gastric acid disorders with rapid onset of action within about 15 minutes to about 120 minutes and preferably within about 15 minutes to about 60 minutes following oral administration without a food effect requirement.

It is another object of the present invention to provide a dosage form combining a proton pump inhibitor with a cholinergic agonist and an antacid, like an aluminum, magnesium or calcium salt of an antacid, in which there is an effective gastric acid suppressing amount of proton pump inhibitor to treat and/or prevent gastric acid disorders with rapid onset of action within about 15 minutes to about 120 minutes and preferably within about 15 minutes to about 60 minutes following oral administration without a food effect requirement, and an effective amount of antacid to provide rapid, immediate, temporary and sustained relief from common or episodic heartburn, including pain, discomfort and other symptoms associated therewith, for about 24 to about 72 hours or more. It is also an object of the present invention to instruct a patient in need of gastric acid disorder therapy to take, without food or on an empty stomach, a dosage form comprised of a proton pump inhibitor, a cholinergic agonist and an antacid, like an aluminum, magnesium or calcium salt of an antacid at any time of the day or night, possibly even on demand or as needed, to provide rapid, immediate, temporary and sustained relief from symptoms associated with the gastric acid disorder. Finally, it is an object of the present invention to instruct a patient in need of gastric acid disorder therapy to take, without food or on an empty stomach, a dosage form comprised of a proton pump inhibitor and a cholinergic agonist with an antacid, like an aluminum, magnesium or calcium salt of an antacid, or individual dosages of a proton pump inhibitor, a cholinergic agonist and an antacid, on demand or as needed at any time of the day or night to provide rapid, immediate, temporary and sustained relief from symptoms associated with the gastric acid disorder.

In accordance with this object, a PPI, a cholinergic agonist and an antacid may be administered together as a single unitary dose in the form of a liquid or solid, or administered together, but separately as either liquids or solids or a combination thereof, and may be administered on an as-needed basis. For example, in one dosage form contemplated by the present invention, the antacid and cholinergic agonist may be formulated into a separate chewable tablet taken concomitantly with a non-chewable PPI tablet or capsule formulated to prevent premature luminal acid activation. The oral medications when formulated as a single unitary dose may include other additives, such as, for example, parietal cell activators, antiflatulents, flavorings, sweeteners and the like.

These and other objects, features, and advantages of the present invention may be better understood and appreciated from the following detailed description of the embodiments thereof, selected for purposes of illustration and shown in the accompanying figures and examples. It should therefore be understood that the particular embodiments illustrating the present invention are exemplary only and not to be regarded as limitations of the present invention.

### BRIEF DESCRIPTION OF THE DRAWINGS

The foregoing and other objects, advantages and features of the invention, and the manner in which the same are accomplished, will become more readily apparent upon consideration of the following detailed description of the invention taken in conjunction with the accompanying Figures and Examples, which illustrate exemplary embodiments, wherein:

FIG. 1 illustrates drug distribution models;

FIG. 2 shows the rapid onset of a PPI, i.e., omeprazole, when concomitantly administered with a cholinergic agonist, i.e., carbachol, without food; and

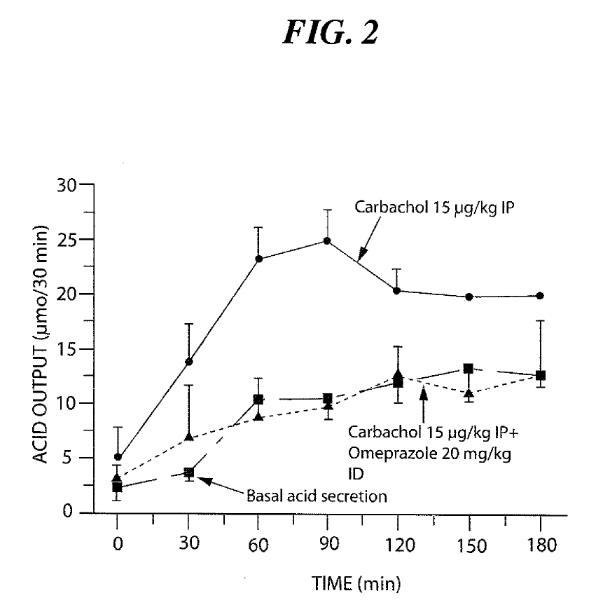

FIG. 3 depicts a cross-section of an exemplary single unitary oral dosage form, i.e., a tablet, formulated with a proton pump inhibitor and a cholinergic agonist, in accordance with the present invention

### DETAILED DESCRIPTION OF THE INVENTION

By way of illustrating and providing a more complete appreciation of the present invention and many of the attendant advantages thereof, the following detailed description and examples are given concerning the novel methods, instructions, combinations and compositions to prevent and/or treat gastrointestinal disorders.

In general, the present invention employs a pharmaceutical combination comprising a safe and effective amount of a proton pump inhibitor and a cholinergic agonist to treat or prevent a gastrointestinal disorder. While the present invention may be embodied in many different forms, several embodiments are discussed herein with the understanding that the present disclosure is to be considered only as an exemplification of the principles and teachings of the present invention, and it is not intended to limit the present invention to the embodiments described or illustrated.

The phrase “safe and effective amount(s)”, as used herein, means an amount of a proton pump inhibitor and an amount of a cholinergic agonist, when used in combination with one another according to the teachings of the present invention, that is sufficient to significantly and positively or therapeutically modify or prevent the condition to be treated consistent with the objectives of the present invention, without causing serious or otherwise treatment-limiting side effects (at a reasonable benefit/risk ratio), within the scope of sound medical judgment. The safe and effective amount of the drugs utilized in accordance with the present invention will vary with the particular gastrointestinal disorders, conditions and/or symptoms being treated, the age, weight and physical conditions of the patients being treated, the severity of the gastrointestinal disorders, conditions and/or symptoms, the duration of treatments, the nature of concurrent therapies, the specific drugs and dosage forms employed, the particular pharmaceutically-acceptable carriers utilized, and like factors within the knowledge and expertise of the attending physicians.

The terms “gastrointestinal disorder(s)”, “GI disorder(s)”, “gastric acid disorder(s)” and “gastrointestinal issue(s)” are used herein broadly and interchangeably and encompass any disease, illness, sickness, disorder, condition, symptom or issue involving or concerning any part or portion of the upper gastrointestinal tract of an animal, including a human, that is characterized by, relates to, arises out of or results from abnormalities in gastric acid secretion. The term “upper gastrointestinal tract”, as used herein, is defined to include the esophagus, the stomach, the duodenum, and the jejunum. Such gastrointestinal disorders include, for example: disorders not necessarily manifested by the presence of ulcerations in the gastric mucosa (herein, “non-ulcerative gastrointestinal disorders”), including chronic or atrophic gastritis, non-ulcer dyspepsia, gastroesophageal reflux disease (GERD), poorly responsive systematic GERD, esophagitis, mild distal esophagitis, heartburn, nocturnal heartburn, episodic heartburn, nocturnal acid breakthrough, and gastric motility disorders; “peptic ulcer diseases”, e.g., gastric, duodenal and jejunal ulcers; and other disorders, such as abnormal gastric acid secretion, severe erosive esophagitis, pathological gastrointestinal hypersecretory disease such as Zollinger Ellison syndrome, ulcerated dyspepsia, abdominal pain, abdominal discomfort, esophageal pain, esophageal discomfort, gastroesophageal reflux, gastroparesis, acid-related asthma, cough or apnea, gastroesophageal reflux with pyrosis and the like. Gastrointestinal disorders especially refer to and include disorders of the upper gastrointestinal tract that are conventionally treated with antacids, PPIs and H₂ receptor-antagonist anti-secretory agents alone or in combination. It is currently believed that these conditions are caused by an imbalance between aggressive factors, namely acid and pepsin production, and defensive factors referred to as mucus, bicarbonate and prostaglandin production. These above-listed conditions commonly arise in healthy, pre-disposed or critically ill patients, and may be accompanied by significant upper gastrointestinal bleeding and other symptoms.

The term “episodic heartburn,” as used herein, means pain or the sensation of burning under the sternum (breastbone) generally caused by regurgitation or backflow (reflux) of acidic juices from the stomach into the esophagus thereby irritating the esophagus, and usually, but not necessarily, it is associated with the ingestion of different foods. Also included in this definition of “episodic heartburn” is sour stomach, indigestion and water brash/regurgitation. The term “nocturnal heartburn” is reflux symptoms at night. The term “nocturnal acid breakthrough” (NAB) is a physiologic phenomenon that generally occurs in the stomach. It is not necessarily associated with esophageal symptoms, but rather is generally defined as a drop in intragastric pH to below 4 for at least 1 hour overnight in patients taking proton pump inhibitor therapy and it is believed to occur in over 70% of patients with all the proton pump inhibitors currently available. It typically occurs about 6 to 7 hours after the second dose of the proton pump inhibitor, and this drop in pH to below 4 can last for as many as 3 to 4 hours in some patients.

The terms “treat(s)”, “treated”, “treating” or “treatment” are used herein interchangeably and refer to any treatment of a disorder in an animal diagnosed or inflicted with such disorder and includes, but is not limited to: (a) caring for an animal diagnosed or inflicted with a disorder; (b) curing or healing an animal diagnosed or inflicted with a disorder; (c) causing regression of a disorder in an animal; (d) arresting further development or progression of a disorder in an animal; (e) slowing the course of a disorder in an animal; (f) relieving, improving, decreasing or stopping the conditions of a disorder in a animal; (g) relieving, decreasing or stopping the symptoms caused by or associated with a disorder in an animal; or (h) reducing the frequency, number or severity of episodes caused by or associated with a disorder in an animal.

The terms “prevent(s)”, “prevented”, “preventing” or “prevention” are used herein interchangeably and refer to any prevention or any contribution to the prevention of a disorder in an animal or the development of a disorder if none has occurred in an animal which may be predisposed to such disorder but has not yet been inflicted with or diagnosed as having such disorder.

It should be understood that the term “about” as used herein means approximately or near or around. For example, when the term “about” is used in relation to a specified dosage amount or range, the term “about” indicates that the dosage amount or range specified is an approximate dosage amount or range and that it includes not only the amount or range actually specified, but those amounts or ranges that may also be safe and effective that are somewhat outside the cited amount or range.

As used herein, the terms “comprising,” “comprises”, “comprised of,” “Including,” and “such as” are used in their open, non-limiting sense.

As previously indicated herein, the novel pharmaceutical compositions of the present invention comprise, in safe and effective amounts, at least one proton pump inhibitor and at least one cholinergic agonist to effectively stimulate or activate the parietal cells following oral administration to induce rapid onset of proton pump inhibitor action, without a need for a food effect or delay in onset of proton pump inhibitor action usually experienced when proton pump inhibitor therapy is first initiated.

The terms “proton pump inhibitor(s)” and “PPI(s)” are used herein interchangeably and refer to any agent, drug, compound or substance which suppresses or interferes with gastric acid secretion by inhibition of the H+/K+ ATPase enzyme system of the parietal cells. Without wishing to be bound by any specific mechanism of action or theory, it is thought that PPIs block the final step of gastric acid production in the H+/K+ ATPase enzyme system with regard to both basal and stimulated acid secretion irrespective of the stimulus.

Examples of proton pump inhibitors contemplated for use in the present invention include, but are not limited to, dontoprazole, esomeprazole, habeprazole, hydroxyomeprazole, lansoprazole, leminoprazole, pantoprazole, pariprazole, perprazole, (s-omeprazole magnesium) omeprazole, omneirazole, rabeprazole, ransoprazole, tenooprazole, TU-199 and mixtures thereof in neutral form, as well as the pharmaceutically acceptable salt, prodrug, derivative, R- or S-enantiomer, isomer, free base, anhydrate, hydrate, solvate, polymorph or combinations thereof, whether in crystalline form, amorphous form or a combination thereof, of such proton pump inhibitor. Further examples of proton pump inhibitors, related compounds and analogs thereof, that are contemplated by the present invention, are described in Remington: The Science and Practice of Pharmacy, Lippincott Williams & Wilkins, Philadelphia, 20th Edition, Chapter 66, pp. 1225-1226 (Mack Publishing Company, 2003), Sjostrom, J -E et al.: Antimicrobial Agents and Chemotherapy, 41(8):1797-1801 (August, 1997), Massoomi, F et al.: Omeprazole: a comprehensive review. Pharmacotherapy, 13(1):46-59 (1993), Maton, P N: Omeprazole. N Engl J Med., 324(14):965-975 (1991), Lampkin T A et al.: Omeprazole: a novel antisecretory agent for the treatment of acid-peptic disorders. DICP Ann Pharmacother., 24:393-402 (1990), WO 2004/062695, WO 2004/060372, WO 2004/004719, WO 2004/004690, WO 2001/91748, WO 2001/51050, WO 2000/50038, U.S. Pat. Nos. 4,628,098, 4,689,333, 4,738,974, 4,758,579, 4,786,505, 4,853,230, 5,013,743, 5,026,560, 5,035,899, 5,045,321, 5,093,132, 5,433,959, 5,690,960, 5,714,504, 5,877,192, 5,900,424, 6,147,103, 6,150,380, 6,166,213, 6,191,148, 6,369,085 and 6,428,810, JP-A-52-62275, JP-A-54-141783 JP-A-57-53406, JP-A-58-135881, JP-A-58-192880, JP-A-59-181277, JP-A-61-50978, JP-A-62-116576, JP-A-62-277322, JP-A-62-258320, JP-A-62-258316, JP-A-64-6270, JP-A-64-79177, JP-A-5-59043, JP-A-62-111980, JP-A-5-117268, EP-A-166287 and EP-A-519365, all of which are incorporated herein by reference in their entireties.

The term “derivative” as used herein means a chemically modified compound wherein the chemical modification takes place at one or more functional groups of the compound and/or on an aromatic ring, when present. The derivative however, is expected to retain the pharmacological activity of the compound from which it is derived.

The term “pharmaceutically acceptable salt” refers to a non-toxic salt that retains or does not interfere with the biological effectiveness or properties of the free acid and/or base of the specified compound. Examples of pharmaceutically acceptable salts include sulfates, hydrosulfates, bisulfate's, sulfites, bisulfate's, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phylacetates, phenylpropionates, phenylbutyrates, citrates, lactates, gamma-hydroxybutyrates, glycollates, tartarates, methane-sulfonates, propanesulfonates, naphthalene-1-sulfonates, naphthalene-2-sulfonates, and mandelates. Several of the officially approved salts are listed in Remington: The Science and Practice of Pharmacy, Lippincott Williams & Wilkins, Philadelphia, Easton, 21st Edition, Chapters 17 and 18, pp. 231-265 (Mack Publishing Company, 2005).

If a compound of the invention is a base, a desired salt may be prepared by any suitable method known to the art, including treatment of the free base with an inorganic acid such as hydrochloric acid, hydrobromic acid, sulfuric acid, nitric acid, phosphoric acid, and the like; or with an organic acid such as acetic acid, maleic acid, succinic acid, mandelic acid, fumaric acid, malonic acid, pyruvic acid, oxalic acid, glycolic acid, salicylic acid, a pyranosidyl acid such as glucuronic acid or galacturonic acid, an alpha-hydroxy acid such as citric acid or tartaric acid, an amino acid such as aspartic acid or glutamic acid, an aromatic acid such as benzoic acid or cinnamic acid, a sulfonic acid such as p-toluenesulfonic acid or ethanesulfonic acid; or the like.

If a compound of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative examples of suitable salts include organic salts derived from amino acids such as glycine and arginine; ammonia; primary amines; secondary amines; tertiary amines; and cyclic amines such as piperidine, morpholine, and piperazine; as well as inorganic salts derived from sodium, calcium, potassium, magnesium, manganese, iron, copper, zinc, aluminum, and lithium.

In the case of compounds, salts or solvates that are solids, it is to be understood by those versed in this art that the compounds, salts and solvates may exist in different crystal forms, all of which are intended to be within the scope of the present invention and specified formulas. Pharmaceutical compounds may exist as single geometric isomers, stereoisomers, racemates, and/or mixtures of enantiomers and/or diastereomers. All such single geometric isomers, stereoisomers, racemates and mixtures thereof are contemplated by the present invention.

It should be understood by those skilled in this field that the proton pump inhibitors may be used in any satisfactory form as indicated above, e.g., in the form of its racemate or a single enantiomer or polymorph, in the non-salt form or in the form of an alkaline salt of the racemate or one of its single enantiomers or polymorphs. However, because proton pump inhibitors are susceptible to degradation and/or transformation in acid reacting and neutral media, it is believed that they should be protected from contact with acid gastric juice by any suitable means, such as an enteric coating, a modified release coating, a protective layer and/or a buffering agent, to ensure that the objectives of the present invention are accomplished. While any effective PPI is contemplated by the present invention, it is believed that omeprazole, esomeprazole, rabeprazole, pantoprazole and lansoprazole and, in particular, the salts thereof or the (s)-isomers of omeprazole, esomeprazole, rabeprazole, pantoprazole and lansoprazole in the form of salts, are preferred PPIs.

The term “cholinergic agonist(s)”, as used herein, refers to any compound, agent, drug or substance that includes but is not limited to: (1) has the ability to activate the parietal cells to produce hydrogen ion or gastric acid to a level sufficient to induce rapid PPI onset of action, especially when simultaneously administered or co-administered with a PPI; and (2) (a) can release, liberate, resemble, mimic, mediate or be activated by acetylcholine or a related compound, (b) has physiological effects similar to those of acetylcholine or a related compound, (c) can stimulate nerve systems activated by acetylcholine or a related compound, (d) is a cholinomimetic substance with direct action, (e) is an anticholinesterase agent, (f) is a muscarinic agonist, and/or (g) is a cholinergic releasing agent. It should therefore be understood by those who are versed in this art that the term cholinergic agonist, as used herein, is to be broadly construed and includes muscarinic agonists, cholinesterase inhibitors and acetylcholine releasing agents.

A cholinergic agonist in accordance with the present invention may be used in any suitable chemical form, including salts thereof, and in any suitable dosage form, including granules, beads, powders, particles, liquids, tablets, capsules, caplets, injectables, etc. Thus, like the PPIs, it should be understood by those skilled in this field that the cholinergic agonists may be used, e.g., in the form of its racemate or a single enantiomer or polymorph, in the non-salt form or in the form of a salt of the racemate or one of its single enantiomers or polymorphs.

Examples of cholinergic agonists contemplated by the present invention include but are not limited to choline esters, such as methacholine, carbachol, bethanecol and their salts, such as methacholine chloride, carbachol chloride and bethanecol chloride; natural and synthetic alkaloids, such as arecholine, aceclidine, acetylcholine, pilocarpine, muscarine, acyclidine, oxotremorine and their salts, such as arecoline HBr, acetylcholine chloride, pilocarpine HCl,(+)-, muscarine chloride(+)- and oxotremorine sesquifimarate; reversible anticholinesterase agents, such as physostigmine, neostigmine, edrophonium, pyridostigmine, demecarium, ambenonium and their salts, such as neostigmine bromide, neostigmine methylsulfate, pyridostigmine bromide, physostigmine salicylate, physostigmine sulfate and demecarium bromide; and other cholinergic agonists, such as cis-2-methyl-5-trimethylammoniummethyl-1,3-oxathiolane iodide (OXA-22), McN-A-343, CL-1017, RS-86 (2-ethyl-8-methyl-2,8-diazospiro-4,5-decan-1,3-dianhydrobromide), AF102B [±cis-2-methyl-spiro(1,3-oxathiolane-5,3′) quinuelidine], azaspirodecanes (2-methyl-1,3-dioxaazaspiro[4,5]-decanes), tetrahydroaminoacridine, HP 029, galanthamine, 9-Amino-1,2,3,4-tetrahydroaminoacridine (THA), linopirdine [DuP 996; 3,3-bis(4-pyrindinylmethyl)-1-phenylindolin-2-one], HP 749 [N-(n-propyl)-N-(4-pyridinyl)-1H-indol-1-amine], dexpanthenol; echothiophate iodide, isofluorophate, cis-dioxolane,(+)-, (4-hydroxy-2-butynyl)-1-trimethylammonium m-chlorocarbonilate chloride, talsaclidine, cevimeline, McN-A343, nicotine tartrate, S(−)-, isofluorophate, demecarium, echothiophate and their salts. While any effective cholinergic agonist is contemplated by the present invention, it is believed that bethanecol, pilocarpine and carbachol are preferred cholinergic agonists. See Remington: The Science and Practice of Pharmacy, Lippincott Williams & Wilkins, Philadelphia, 21st Edition, Chapter 71, pp. 1389-1398 (Mack Publishing Company, 2005), which is incorporated herein by reference in its entirety.

Further examples of cholinergic agonists contemplated by the present invention are described in, for example, EP Patent No. EP0629399, WO 2004/052365 and WO 2001/89526, all of which are incorporated herein by reference in their entireties.

The term “muscarinic agonist”, as used herein, refers to any compound, agent, drug or substance that binds to and/or activates muscarinic cholinergic receptors. Examples of muscarinic agonists include muscarine, bethanecol, milameline, xanomeline, sabcomeline, arecoline, oxotremorine, pilocarpine, CL-1017, RS-86 (2-ethyl-8-methyl-2,8-diazospiro-4,5-decan-1,3-dianhydrobromide), AF102B [±cis-2-methyl-spiro(1,3-oxathiolane-5,3′)quinuclidine], talsaclidine, cevimeline, McN-A343, azaspirodecanes (2-methyl-1,3-dioxaazaspiro[4,5]-decanes) and their salts. See, for example, Experimental Therapeutics, Deborah B. Marin and Kenneth L. Davis 2000 at http://www.acnp.org/g4/GN401000136/CH133.html, Bikadi, Z. and Simonyi, M.: Current Medicinal Chemistry, 10:1241-1253 (2003); U.S. Pat. Nos. 4,855,290; 5,340,821, 5,580,880, 5,908,866, 5,679,713, 6,602,891; 6,369,081; 6,376,675; 6,211,204; 6,096,767; US Patent Publication Nos. US2003032658; US2001036953, WO 2002/060444, WO 2002/064124, WO 1993/03718, WO 2002/003684, WO 1998/46231, WO 1998/46232, WO 1998/30243, WO 1999/47171, WO 2001/85145, all of which are incorporated herein by reference in their entireties.

The term “cholinesterase inhibitor”, as used herein, refers to any compound, agent, drug or substance which acts to inhibit acetylcholinesterase or butyrylcholinesterase, an enzyme which breaks down acetylcholine or butrylcholine, respectively, and thus enhances and subsequently prevents transmission of nerve impulses from one nerve cell to another or to a muscle Examples of cholinesterase inhibitors contemplated by the present invention include, but are not limited to, donepizil (Aricept), tacrine (Cognex), rivastigmine (Exelon), galantamine (Reminyl), physostigmine (Synapton), metrifonate (Promem), quilostigmine, tolserine, thiatolserine, cymserine, thiacymserine, neostigmine, eseroline, zifrosilone, mestinon, huperzine A, icopezil, pyridostigmine, ambenonium, neostigmine, tacrine, tetrahydroaminoacridine, HP 029, 9-Amino-1,2,3,4-tetrahydroaminoacridine (THA) and pharmaceutically acceptable salts thereof. Further examples of cholinesterase inhibitors contemplated by the present invention are described in U.S. Pat. Nos. 5,104,880, 5,171,750, U.S. Pat. Nos. 5,574,046, 5,750,542, 6,759,552 and 6,706,741, U.S. patent application Ser. No. 07/639,614 filed Jan. 10, 1991 and U.S. patent application Ser. No. 07/676,918 filed Mar. 28, 1991, WO 98/30243, WO 93/06105, WO 93/05779, WO 00/30446, WO 01/84141 and WO 00/07600, and US Patent Publication No. US2004087658, all of which are incorporated herein by reference in their entireties.

The term “cholinergic releasing agent”, as used herein, refers to any compound, agent, drug or substance that acts to release acetylcholine and/or enhance stimulus-induced acetylcholine delivery into the synapse. Examples of cholinergic releasing agents contemplated by the present invention include, but are not limited to, linopirdine [DuP 996; 3,3-bis(4-pyrindinylmethyl)-1-phenylindolin-2-one] and HP 749 [N-(n-propyl)-N-(4-pyridinyl)-1H-indol-1-amine] and pharmaceutically acceptable salts thereof. Further examples of cholinergic releasing agents contemplated by the present invention are described in WO9630372 and WO9640054, which are incorporated herein by reference in their entireties.

As indicated above, safe and effective amounts of a proton pump inhibitor and a cholinergic agonist that are administered to a subject will be dependent on, inter alia, the body weight of the subject and the therapeutic effect to be achieved. Illustratively, when the drug is a PPI such as, for example, esomeprazole, lansoprazole, leminoprazole, omeprazole, pantoprazole, pariprazole or rabeprazole, and the subject is an infant or a small animal, such as a cat, rabbit, dog or sheep, for example, a relatively low amount of the drug in the dose range of from about 1 mg to about 20 milligrams (hereinafter “mg”) is likely to provide blood serum concentrations consistent with therapeutic effectiveness. However, when the subject is a human adult or a larger animal, such as a cow, a horse and equine animals and livestock, achievement of therapeutic blood serum concentrations of the drug are likely to require dosages containing a relatively greater amount of the drug. For example, for a human adult, the methods, kits, combinations, instructions and compositions of the present invention comprise a PPI, for example, omeprazole, lansoprazole, pantoprazole, rabeprazole, esomeprazole, pariprazole or leminoprazole, in a dosage range of from about 1 mg to about 1000 mg or more, and a cholinergic agonist, such as bethanecol, carbachol, pilocarpine, pyridostigmine or ambenonium, in a dosage range of from about 1 mg to about 250 mg or greater.

The pharmaceutical compositions of the present invention can be administered by any suitable means to subjects, preferably orally or enterally to the subjects. This can be accomplished, for example, by administering to the subject a solid or liquid oral dosage form by mouth or via a gastric feeding tube, a duodenal feeding tube, a nasogastric (ng) tube, a gastrostomy, or other indwelling tubes placed in the GI tract. The oral pharmaceutical compositions of the present invention are generally in the form of individualized or multi unit doses, such as tablets, powders, suspension tablets, chewable tablets, rapid melt tablets, capsules, effervescent powders, effervescent tablets, pellets, granules, liquids, solutions, or suspensions, respectively. The oral pharmaceutical compositions may contain a PPI in an amount of from about 1 mg or less to about 1000 mg or more, or preferably from about 5 mg to about 300 mg, or preferably from about 10 mg to about 120 mg, or preferably from about 15 mg to about 80 mg, and a cholinergic agonist in a dosage amount of from about 0.1 mg to about 500 mg or greater, or preferably from about 1 mg to about 250 mg, or more preferably from about 1 mg to about 100 mg, or even more preferably from about 2.5 mg to about 50 mg, or even more preferably from about 5 mg to about 50 mg, or even more preferably from about 10 mg to about 50 mg. By way of example, an oral unit dose or composition of the present invention may contain a PPI in a dosage amount of about 5 mg, about 10 mg, about 15 mg, about 20 mg, about 25 mg, about 30 mg, about 40 mg, about 50 mg, about 60 mg, about 75 mg, about 80 mg, about 100 mg, about 250 mg or about 300 mg, and a cholinergic agonist in a dosage amount of about 1 mg, about 2.5 mg, about 3 mg, about 5 mg, about 7.5 mg, about 10 mg, about 15 mg, about 25 mg, about 30 mg, about 40 mg, about 50 mg, about 60 mg, about 75 mg, about 100 mg, about 150 mg, about 180 mg, about 200 mg or about 250 mg. Of course, it should be appreciated that a particular unit dosage form and amount can be selected to accommodate the desired frequency of administration used to achieve a specified daily dosage and therapeutic effect as indicated above.

As stated hereinabove, proton pump inhibitors have been employed in the past, in the form of modified release, immediate release or enteric coated, for the treatment of gastric acid disorders, which condition is characterized by abnormal acid production or excess gastric acid. According to one aspect of the present invention, a modified release or enteric coated proton pump inhibitor further coated or blended with an immediate release coating of a cholinergic agonist. By “modified release” or “enteric coated”, it is understood to mean a composition which when orally administered to a patient to be treated, the active ingredient like a PPI will be released for absorption into the blood stream in the intestine, preferably the small intestine, following oral administration. For example, it is preferred that approximately 100 percent of the PPI, when in modified release or enteric coated form, will be released for absorption into the blood stream within about 5 minutes to about 90 minutes or later following ingestion.

While the proton pump inhibitor can be released from the pharmaceutical composition in a sustained release or enteric coated manner, the cholinergic agonist can be formulated for immediate or modified release following ingestion. By “immediate release,” it is understood to mean that the drug, which when orally administered to a subject to be treated, will be released immediately and completely from the composition for absorption into the blood stream within about 1-60 minutes following ingestion.

As indicated above, pharmaceutical compositions according to the present invention will employ an effective gastric acid suppressing amount of a proton pump inhibitor and an effective parietal cell activation amount of a cholinergic agonist. By “effective gastric acid suppressing amount”, it is understood to mean any safe and effective amount which when administered to a subject to be treated, will achieve a beneficial pharmacological effect or therapeutic improvement without undue adverse or treatment limiting side effects. In the case of proton pump inhibitors, an effective gastric acid suppressing amount may be, for example, an amount that provides some level of inhibition of the H+/K+ ATPase enzyme system, e.g., at the secretory surface of the parietal cells, that is recognized in the art to be therapeutically effective. The beneficial effect will also include some decrease in gastric acid secretion or output by the parietal cells for an extended period of time. Exemplary effective gastric acid suppressing amounts of a PPI include those amounts mentioned herein above, administered one or more times per day according to the present invention, as will be more fully describe herein below.

By “effective parietal cell activation amount”, it is understood to mean any safe and effective amount which when administered to a subject to be treated, will achieve a beneficial pharmacological effect or therapeutic improvement without undue adverse or treatment limiting side effects. In the case of a cholinergic agonist, an effective parietal cell activation amount may be, for example, an amount that provides some level of parietal cell activation that is therapeutically effective, i.e., to produce hydrogen ion or gastric acid to a level sufficient to induce rapid PPI onset of action following PPI absorption and accumulation without a food effect, especially when simultaneously administered or co-administered with a PPI. The beneficial effect will also include some initial increase in gastric acid secretion or output by the parietal cells to induce PPI onset of action at anytime of the day without a food effect requirement. Exemplary effective parietal cell activating amounts of a cholinergic agonist include those amounts mentioned herein above. Alternatively, an exemplary effective parietal cell activating amount of a cholinergic agonist may be from about 10 μg/kg to about 1000 μg/kg administered one or more times a day. These amounts will of course vary, dependent upon a number of variables, including the psychological needs of the patient to be treated, the dosage form selected and the particular PPI and cholinergic agonist used.

Consistent with the present invention, these and other dosage forms discussed herein may be administered to individuals on a regimen of one, two or more doses per day, at any time of the day on demand or whenever needed.

In order to more finely regulate the dosage schedule, the active substances may be administered separately in individual dosage units at the same time or carefully coordinated times within about 60 minutes or less of another. Thus, the respective substances can be individually formulated in separate unit dosage forms in a manner similar to that described above or herein.

Combinations of a proton pump inhibitor and a cholinergic agonist in the same pharmaceutical are more convenient and are therefore preferred, especially in the coated tablet or caplet form for oral administration. Alternatively, however, the pharmaceutical combinations of the present invention may comprise two distinct oral dosage forms that may be administered concomitantly, where each oral dosage form is formulated as an enteric coated dosage form or for modified or immediate release.

Optionally, the oral pharmaceutical combinations of the present invention may include other active ingredients. In addition, the present invention contemplates that other active ingredients may be administered concurrently with the pharmaceutical combinations of the present invention. Examples of other active ingredients include parietal cell activators, antacids and antiflatulents (e.g., simethicone 80 mg, Mylanta Gas Relief Formula®, Phazyme®). Specific examples of parietal cell activators include, but are not limited to, lecithin, choline, protein powders such as, protein hydrosylates, protein powders such as whey, milk and egg protein powders, chocolate, sodium bicarbonate, calcium (e.g., calcium carbonate, calcium gluconate, calcium hydroxide, calcium acetate and calcium glycerophosphate), peppermint oil, spearmint oil, coffee (even if decaffeinated), tea and colas, caffeine, theophylline, theobromine, and amino acids (particularly aromatic amino acids such as phenylalanine and tryptophan) and combinations thereof and the salts thereof.

The term “antacid (s)” a used herein, refers to any compound, which reacts with hydrochloric acid to form salt and water. Antacid agents are fully described in the following publications which are incorporated herein by reference in their entireties: G. B. 925,001, to Fielding et al., published on May 1, 1963, and Remington: The Science and Practice of Pharmacy, Lippincott Williams & Wilkins, Philadelphia, Easton, 21st Edition, Chapter 24, pp. 382 and Chapter 66, pp. 1294-1317 (Mack Publishing Company, 2005), both of which are incorporated herein by reference in their entireties.

Specific examples of antacids contemplated herein include, but are not limited to, aluminum carbonate, aluminum hydroxide, aluminum phosphate, aluminum hydroxy-carbonate, dihydroxy aluminum sodium carbonate, aluminum magnesium glycinate, dihydroxy aluminum amino acetate, dihydroxy aluminum aminoacetic acid, calcium carbonate, calcium phosphate, aluminum magnesium hydrated sulfates, magnesium aluminate, magnesium alumino silicates, magnesium carbonate, magnesium glycinate, magnesium hydroxide, magnesium oxide, magnesium trisilicate, sucrafalte, sodium bicarbonate, and mixtures thereof.

In formulating the compositions, the active substances, in the amounts described above, are compounded according to accepted pharmaceutical practice with pharmaceutically acceptable diluents, binders, vehicles, carriers, excipients, binders, disintegrating agents, lubricants, swelling agents, permeabilizing agents, plasticizers, solubilizing agents, wicking agents, cooling agents, preservatives, stabilizers, sweeteners, flavors, etc., in the particular type of unit dosage form.

Examples of excipients include acacia, alginic acid, croscarmellose, gelatin, gelatin hydrosylate, mannitol, plasdone, sodium starch glycolate, sorbitol, sucrose, and xylitol. For molded or compressed tablet formulations, suitable excipients that may be used include amorphous lactose, beta lactose, microcrystalline cellulose, croscarmellose sodium, dicalcium phosphate, carboxymethyl cellulose, hydroxypropyl cellulose, polyethylene gylcols, sodium lauryl sulfate, and the like.

Examples of stabilizers or preservatives include, for example parahydroxybenzoic acid alkyl esters, antioxidants, antifungal agents, and other stabilizers/preservatives known in the art.

Examples of coloring agents” include, for example, water soluble dye, Lake dye, ion oxide, natural colors, titanium oxide, and the like.

Examples of diluents or fillers include water-soluble and/or water-insoluble tableting fillers. The water-soluble diluent agent may be constituted from a polyol of less than 13 carbon atoms, in the form of directly compressible material (the mean particle size being between about 100 and about 500 microns), in the form of a powder (the mean particle size being less than about 100 microns) or a mixture thereof. Other examples of water soluble diluents include protein powders such as whey protein powder, milk protein powder and egg protein powder. The polyol is preferably chosen from the group comprising of mannitol, xylitol, sorbitol and maltitol. The water-insoluble diluent agent may be a cellulosic derivative preferably microcrystalline cellulose.

Examples of disintegrating agents include, but are not limited to, crosslinked sodium carboxymethylcellulose, crospovidone and their mixtures. A part of the disintegrating agent may be used for the preparation of PPI, cholinergic agonist, parietal activator and/or antacid granules.

Examples of lubricating agents include, but are not limited to, magnesium stearate, stearic acid and its pharmaceutically acceptable alkali metal salts, sodium stearylfumarate, Macrogol 6000, glyceryl behenate, talc, colloidal silicon dioxide, calcium stearate, sodium stearate, Cab-O-Sil, Syloid, sodium lauryl sulfate, sodium chloride, magnesium lauryl sulfate, talc and their mixtures. A part of the lubricant may be used as an internal solid lubricant, another part may be sprayed over the outer surface of the tablet.

Examples of swelling agents include, but are not limited to, starch, polymers such as microcrystalline cellulose, hydroxypropylmethyl cellulose, sodium carboxymethylcellulose and ethylcellulose, waxes such as bees wax, and natural materials such as gums and gelatins, or mixtures of any of the above.

Examples of permeabilizing agents include, but are not limited to, silica having a high affinity with aqueous solvents, such as SyloidD®, maltodextrines, beta-cyclodextrines and their mixtures. The permeabilizing agents enable creation of a hydrophilic network that enhances the penetration of the saliva and the disintegration of the tablet. A part of a permeabilizing agent may be used for the preparation of the PPI, cholinergic agonist, parietal cell activator and/or antacid granules.

Specific examples of plasticizers include, but are not limited to, benzyl benzoate, chlorobutanol, dibutyl sebacate, diethyl phthalate, glycerin, mineral oil and lanolin alcohols, petrolatum and lanolin alcohols, polyethylene glycol, propylene glycol, sorbitol, triacetin and triethyl citrate.

Additional illustrations of adjuvants which may be incorporated in the tablets are the following: a binder such as gum tragacanth, acacia, corn starch, potato starch, alginic acid, povidone, acacia, alginic acid, ethylcellulose, methylcellulose, microcrystalline cellulose, a derivatized cellulose, such as carboxymethyl cellulose, sodium carboxymethyl cellulose, hydroxyethyl cellulose, hydroxypropyl methylcellulose, and hydroxypropyl cellulose, dextrin, gelatin, glucose, guar gum, hydrogenated vegetable oil, type I, polyethylene glycol, lactose, compressible sugars, sorbitol, mannitol, dicalcium phosphate dihydrate, tricalcium phosphate, calcium sulfate dihydrate, maltodextrins, lactitol, magnesium carbonate, xylitol, magnesium aluminium silicate, maltodextrin, methylcellulose, hydroxypropylcellulose, polyethylene polyethylene oxide, polymethacrylates, plasdone, sodium alginate, starch, pregelatinized starch, zein or the like; a sweetening agent such as sucrose, potassium acesulfame, aspartame, lactose, dihydrochalcone neohesperidine, saccharin, sucralose, polyols such as xylitol and maltitol, sodium saccharide, Asulfame-K, Neotame®, glycyrrhizin, malt syrup and combinations thereof; a flavoring such as berry, orange, peppermint, oil of wintergreen, cherry citric acid, tartaric acid, menthol, lemon oil, citrus flavor, common salt, and other flavors known in the art.

The flavoring is advantageously chosen to give a combination of fast onset and long-lasting sweet taste and get a “round feeling” in the mouth with different textures or additives. Cooling agents can also be added in order to improve the mouth feeling and provide a synergy with flavors and sweetness. When the dosage unit form is a capsule, it may contain in addition to materials of the above type a liquid carrier such as a fatty oil. Various other materials may be present as coatings or to otherwise modify the physical form of the dosage unit. For instance, tablets or capsules may be coated with shellac, sugar or both.

Some of the active agents described above form commonly known pharmaceutically acceptable salts, such as alkali metal and other common basic salts or acid addition salts, etc. References to the base agents are therefore intended to include those common salts known to be substantially equivalent to the parent compound.

It should be understood that the phrase “pharmaceutically acceptable” is used adjectivally herein to mean that the modified noun is appropriate for use in a pharmaceutical product.

In carrying out the objective of the present invention, the proton pump inhibitors and/or cholinergic agonists may be formulated into modified release granules, modified release particles, modified release coated particles or modified release beads or pellets according to any method known to the art for the manufacture of pharmaceutical compositions for incorporation into a variety of oral dosage forms suitable for oral use, such as tablets, such as rapidly disintegrating tablets, compression coated tablets, enteric coated tablets, capsules, tablet-filled capsules, caplets, sachets for sprinkle administration, and the like. In addition, the proton pump inhibitors and the cholinergic agonists may be formulated into immediate release granules or immediate release coated or uncoated raw materials for incorporation into the oral dosage forms of the present invention.

Proton pump inhibitor dosage forms contemplated by the present invention include Prilosec®, Prevacid®, Nexium®, Protonix® and Aciphex® capsules, pellets and tablets and their respective strengths. Cholinergic agonist dosage forms for use in accordance with the present invention include the Urecholine®, Duvoid® and Salogen® tablets and their respective strengths, as reported in the FDA Orange Book, which is incorporated herein by reference in its entirety. These PPI dosage forms can be modified consistent with the present invention to include a cholinergic agonist during the formation of the PPI granules or during the manufacture of the tablet blends prior to compression into the tablets to formulate a pharmaceutical combination of the present invention in which the PPI and cholinergic agonist are in a immediate release, modified release or enteric coated form. Alternatively, the PPI tablets or pellets may be coated with a coating containing a cholinergic agonist in immediate release form to formulate a pharmaceutical combination of the present invention in which the PPI is in a modified release or enteric coated form and the cholinergic agonist is in an immediate release form.

The present invention also contemplates other combined dosage forms containing a proton pump inhibitor and a cholinergic agonist. For instance, such combined dosage forms include bilayer or multilayer tablets, caplets, capsules, tablet-filled capsules or sachets containing, for example, immediate release, modified release or enteric coated granules of a proton pump inhibitor and immediate release, modified release or enteric coated granules of a cholinergic agonist. Bilayer or multilayer tablets may be manufactured utilizing techniques well known in this art, such as by lightly prestamping a proton pump inhibitor layer containing immediate release, modified release or enteric coated PPI granules, adding a layer containing a cholinergic agonist either deficient in or containing a modified release agent or enteric coating, and compressing the combined powder to form the bilayer tablet. Optionally, the cholinergic agonist layer may further contain other agents, such as other parietal cell activators, as described and discussed above.

As indicated above, pharmaceutical medications and methods are disclosed for providing immediate, temporary and sustained relief from pain, discomfort or symptoms associated with gastrointestinal disorders, e.g., episodic heartburn, in subjects. The medications comprise a PPI, a cholinergic agonist and an antacid, and may be administered on an as-needed basis in liquid or solid dosage forms.

By the phrase “immediate, temporary and sustained relief,” it is used in a broad sense herein. More particularly, the term “immediate relief” means that relief obtained from pain, discomfort and/or symptoms associated with a GI disorder, such as episodic heartburn, which occurs within about 5-10 minutes following ingestion of the active ingredients or an antacid. “Temporary relief” on the other hand refers to relief from pain, discomfort and/or symptoms associated with episodic heartburn that lasts in duration on the order of between about 30 minutes and 90 minutes after ingestion of the active ingredients or an antacid. With respect to “sustained relief,” it refers to relief obtained from pain, discomfort and/or symptoms associated with episodic heartburn which lasts in duration for over about 4-6 hours following ingestion of the active ingredients or the PPI and cholinergic agonist. It should therefore be appreciated that by the term “immediate and sustained relief,” as used herein, it means immediate, temporary and sustained relief which starts within about 5-10 minutes following ingestion of the active ingredients and continues and remains constant for at least about 6-24 hours after ingestion of the active ingredients; the actual ingredients being an antacid, a PPI and a cholinergic agonist.

The pharmaceutical medications of the instant invention can be conveniently prepared from, for example, commercially available antacids, PPIs and cholinergic agonists and may be formulated into liquid or solid dosage forms or combinations thereof. For example, the pharmaceutical medications may be taken as a single unitary dose containing the antacid, PPI and cholinergic agonist in a liquid or solid dosage form. Likewise, the present invention contemplates taking the ingredients substantially together, but separately in the same or different dosage forms, such as taking the antacid as a liquid dose and the PPI and cholinergic agonist as solid doses or vice versa, or taking them separately as either solid or liquid doses.

When taking the active ingredients substantially together, but separately in same or different dosage forms, the order in which they are ingested is not critical. In other words, the antacid, PPI and cholinergic agonist may be ingested simultaneously, or the antacid may be ingested first followed by the PPI and cholinergic agonist, or the cholinergic agonist may be first ingested followed by the antacid and the PPI, or the PPI may be taken first followed by the cholinergic acid and antacid. It is preferable, however, to formulate the antacids, the PPI and cholinergic agonist into a single liquid or solid unitary dosage form that can be ingested as a single dose or as single liquid mixtures or solid dosage forms which can be co-ingested as single unitary dosages on an as-needed basis, i.e., at or after the onset of pain, discomfort and/or symptoms associated with GI disorders, such as episodic heartburn. When commercially available antacids are selected for use in accordance with the present invention, such as Maalox-Plus®, Mylanta®, Tums®, and Gelusil®, Rolaids®, etc., it is preferable to use the high potency, flavored (mint, cherry, lemon, etc.) liquid antacids, such as, for example, Maalox-Plus® and Mylanta-II®.

By the term “substantially together,” it is meant herein that when the active ingredients, i.e., an antacid, a PPI and a cholinergic agonist, are taken in separate dosage forms, they can be consumed either simultaneously or within a period of time such that the immediate, temporary and sustained relief obtained is constant and uninterrupted. For example, the active ingredients may be taken together or within a few seconds to a few minutes of one another. Nevertheless, it is preferable to ingest a single unitary dose that includes both active ingredients and is in liquid form.

Typical dosages include about 30 mls or 2 tablespoons of a high-potency antacid having an acid-neutralizing capacity equal to the present formulations of, for example, Maalox Plus®, Mylanta-II®. With respect to the PPI, the amount included in the single dosages is believed to be about 10 mg to about 80 mg. For example, a typical dosage amount for providing immediate and sustained relief from episodic heartburn in an adult is about 30 mls of a high potency flavored antacid or the equivalent thereof, about 10 mg to about 1200 mg of omeprazole and about 5 mg to about 50 mg of bethanecol to about 5 mg to about 7.5 mg of pilocarpine administered between about one and about four times per day. Notwithstanding, it should be appreciated that the oral medications of the instant invention are to be taken on an as-needed basis whenever pain or symptoms associated with episodic heartburn is experienced.

Antiflatulents may also be used in combination with the antacids, PPIs and cholinergic agonist in the present invention and include those antiflatulents which are conventionally used in the treatment of gastrointestinal dysfunction, such as, for example, simethicone. Antiflatulents may be used in the present invention in dosage amounts conventionally used in the treatment of gastrointestinal dysfunction.

The pharmaceutical compositions may be in a form suitable for oral use, for example, as tablets, lozenges, aqueous or oily suspensions, dispersible powders or granules, emulsions, hard or soft capsules, syrups or elixirs, as discussed herein. Compositions intended for oral use may be prepared according to any method known to the art for the manufacturer of pharmaceutical compositions and such compositions may contain one or more agents such as, for example, sweetening agents, flavoring agents, coloring agents and the like, in order to provide a pharmaceutically elegant and palatable preparation. Tablets contain the active ingredient in admixture with nontoxic pharmaceutically acceptable excipients that are suitable for manufacture of tablets. These excipients may be inert diluents, for example, calcium carbonate, sodium carbonate, lactose, calcium phosphate or sodium phosphate; granulating and disintegrating agents, for example, maize starch, or alginic acid; binding agents, for example, starch, gelatin or acacia, and lubricating agents, for example, magnesium stearate or stearic acid. The tablets may be uncoated or they may be coated by known techniques to delay disintegration and absorption in the gastrointestinal tract and thereby provide an even longer sustained action over a period of time.

Aqueous suspensions contain the active ingredients in admixture with excipients suitable for the manufacture of aqueous suspensions. Such excipients may be suitable suspending agents, for example, sodium carboxymethyl cellulose, methyl cellulose, hydroxy propyl methyl cellulose, sodium alginate, polyvinylpyrrolidone, gum tragacanth and gum acacia; dispersing or wetting agents may be any suitable naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol, for example, polyoxyethylene sorbitol monnoleate, or condensation product of ethylene oxide with partial esters derived from fatty acids and hexitol and anhydrides, for example, polyoxyethelyne sobirtan monooleate. The aqueous suspensions may also contain one or more suitable preservatives, for example, ethyl, or n-propyl, p-hydroxy benzoate, one or more suitable coloring agents, one or more suitable flavoring agents and one or more suitable sweetening agents, such as sucrose, saccharin, or sodium or calcium cyclamate.

Dispersible powders and granules suitable for preparation of an aqueous suspension by the addition of water provide the active ingredient in admixture with a dispersing or wetting agent, suspending agent and on or more preservatives. Suitable dispersing or wetting agents and suspending agents may be exemplified by those already mentioned above. Additional suitable excipients, for example, sweetening, flavoring and coloring agents, may also be present.

Syrups and elixirs containing the PPI and cholinergic agonist may be formulated with suitable sweetening agents, for example, glycerol, sorbitol, or sucrose. Such formulations may also contain suitable demulcents, preservatives and flavoring and coloring agents.

Examples of different dosage forms, formulations and compositions contemplated by the present invention will now be described.

### Modified Release PPI Compositions

An example of a pharmaceutical composition according to the present invention employs an effective gastric acid suppressing amount of a modified release proton pump inhibitor that has been further coated or blended with an effective parietal cell activation amount of a cholinergic agonist.

Included in a modified release composition according to the present invention is a swelling or modified release agent that is compounded with the PPI, such that when the composition is orally administered to the patient, the swelling agent will swell over time in the patient's gastrointestinal tract, and release the active PPI over a period of time. As is known in the art, such swelling agents and amounts thereof, may be preselected in order to control the time release of the active PPI ingredient. Because the amount of the swelling agent will vary depending upon the nature of the agent, the time release needs of the patient and the like, it is preferred to employ amounts of the agent which will accomplish the objects of the invention.

An exemplary and preferred swelling agent is hydroxy propyl methyl cellulose, in an amount ranging from about 5% to about 50% parts by weight per 100 parts by weight of tablet or formulation. A preferred example will ensure a time release over a period of from about 0.2 hours to about 3.0 hours, more preferably from about 0.2 to about 2.0 hours, and most preferably from about 0.2 to about 1.5 hours.

A binder may also be employed in the present compositions. While any known binding material is useful in the present invention such as those mentioned herein above, it is preferred to employ a material such as one or more of a group of polymers having the repeating unit of 1-ethenyl-2-pyrrolidinone. These polyvinyl pyrrolidinone polymers generally have molecular weights of between about 10,000 and 700,000, and are also known as povidone or PVP.

Amounts of the binder material will, of course, vary depending upon the nature of the binder and the amount of other ingredients of the composition. An exemplary amount of povidone in the present compositions would be from about 1% to about 5% by weight of povidone per 100 parts by weight of the total formulation.

Processing aids such as lubricants, including stearic acid, magnesium stearate, glyceryl behenate, talc and colloidal silicon dioxide, may also be employed, as is known in the art and discussed above. An exemplary amount of a lubricant, such as stearic acid, in the present compositions would be from about 0.5% to about 2.0% by weight per 100 parts by weight of tablet or formulation.

Also in accordance with the present invention, the modified release compositions containing the PPI are preferably coated with a cholinergic agonist for immediate release following oral administration. An exemplary coating in accordance with the present invention comprises a cholinergic agonist, a plasticizer, film forming and/or coating agent and a coloring agent. An exemplary amount of a plasticizer utilized in the coatings of the present invention would be from about 0.01% to about 5% by weight of the tablet.

Specific examples of film forming and/or coating agents include, but are not limited to, carboxymethylcellulose sodium, carnauba wax, cellulose acetate phthalate, cetyl alcohol, confectioner's sugar, ethylcellulose, gelatin, hydroxyethyl cellulose, hydroxy propyl cellulose, hydroxy propyl methyl cellulose, liquid glucose, maltodextrin, methyl cellulose, microcrystalline wax, polymethacrylates, polyvinyl alcohol, shellac, sucrose, talc, titanium dioxide and zein. An exemplary amount of a film forming/coating agent in the present coatings would be from about 0.01% to about 5% by weight of the tablet. Generally speaking to prepare a coating in accordance with the present invention, a cholinergic agonist is suspended or dissolved in an aqueous-solution of polyethlene glycol and hydroxy propyl methyl cellulose and then sprayed on the modified release tablets by a film-coating process to a thickness containing an effective parietal cell activating amount of a cholinergic agonist. Examples of suitable coating thicknesses in accordance with the present invention are from about 0.1 mm to about 2.0 mm or more.

Coated modified release PPI tablets of various sizes can be prepared, e.g., of about 250 mg to about 2000 mg in total weight, containing both of the active substances in the ranges described above, with the remainder being a pharmaceutically acceptable carrier of other materials according to accepted pharmaceutical practice. These coated tablets can, of course, be scored to provide for fractional doses. Gelatin capsules can be similarly formulated.

### Enteric Coated PPI Compositions

In a further embodiment, the pharmaceutical combination of the present invention may be enterically coated to delay disintegration and absorption in the gastrointestinal tract. For example, (1) modified or immediate release PPI granules or modified or immediate release cholinergic agonist granules may be individually enterically coated and compressed to form a tablet or a layer of a bilayer tablet, or (2) the tablet itself or a layer thereof may be coated with an enteric coating.

Enterically coated dosage forms do not necessarily dissolve or become absorbed by humans until they pass through the low pH environment of the stomach and pass into the relatively higher pH of the small intestine. Typical materials conventionally used as enteric coatings include, but are not limited to, cellulose acetate phthalate, polyvinylacetate phthalate, hydroxypropyl methylcellulose phthalate and methacrylic acid-methyl methacrylate copolymers. Such materials can be used individually or in combination. Additional formulating agents, such as plasticizers (e.g., one or more polyethylene glycols or propylene glycol) may be added to ensure physical strength and processability, e.g., to prevent cracking due to stress, low humidity or other factors.

Enterically coated proton pump inhibitors granules can be prepared in a fluid bed granulator by coating or agglomerating PPI powder with one or more enteric coating materials, such that microspheres or small particles of enterically coated proton pump inhibitors are formed. Alternatively, a whole tablet or capsule comprising a proton pump inhibitor and/or a cholinergic agonist can be coated with enteric coating materials. Alternatively, a whole layer comprising a proton pump inhibitor can be coated with enteric coating materials.

Typically, the enteric coating process comprises coating the dosage form with a plurality of layers, e.g., one or two layers or more, of enteric coating material, like a methacrylate polymer such as EUDRAGIT S-100, available from Rohm and Haas, preferably by dipping or spray coating the granules, beads, tablet, caplet, layer or capsule into a freshly prepared solution of the material for about five seconds. The solution of enteric coating material(s) may be prepared by dissolving an appropriate amount of material in, e.g., 100 ml of a 4:6 mixture of acetone and isopropyl alcohol. After each immersion, the coating is allowed to dry in air, e.g., for 30 minutes, prior to the next five-second immersion. A single coating is usually adequate to prevent the coated material from dissolving in the stomach. Alternatively, the granules, beads, tablets, caplets or capsules may be coated or spray-dried in standard coating machines such as those typically employed in the pharmaceutical industry.

According to one embodiment, the proton pump inhibiting agent is prepared in the form of enteric coating layered microgranules consisting of a core comprising the PPI optionally in mixture with an alkaline reacting compound. The core is covered by a separating layer and an enteric coating layer, and the enteric coated microgranules may optionally be over-coated with a barrier coating, such as for instance a methacrylic copolymer-based film, as further discussed below.

The particle size distribution of the enteric coating layered microgranules may be between about 100 microns to about 1200 microns, preferably between about 200 microns and about 500 microns, most preferably around 500 microns. Moreover, the barrier coating is preferably a methacrylic copolymer-based film. This barrier film is preferably obtained from a coating liquid of particles of the copolymers of which at least about 90% of the particles have a particle size of less than about 315 microns. The prepared coating liquid is either water-based or prepared with organic solvents, preferably a water-based dispersion due to environmental concerns. This coating liquid should also be able to be sprayed with conventional spray layering equipment.

The methacrylic copolymer-based barrier coating preferably comprises a butyl methacrylate/(2-dimethylaminoethyl)methacrylate/methyl methacrylate (1:2:1) copolymer. Eudragit® E-PO which is a pH-dependant polymer, is one example of a polymer suitable for use as barrier coating. A barrier coating comprising Eudragit® E-PO may be made mechanically flexible and, when applied in increasing amounts to enteric coating layered proton pump inhibitor microgranules, provide a corresponding increase in the delayed release (dissolution) of the barrier coating. Different times for the delayed dissolution of the barrier coating in a medium of alkaline pH may thus be obtained while maintaining the properties of the enteric coating of the PPI microgranules, i.e., good acid resistance and rapid dissolution in the buffer stage testing at about pH 6.8 of the USP monograph. Eudragit® E-PO is a methacrylate copolymer obtained from Eudragit® E 100 by milling, yielding a fine powder presentation. The barrier coating can also comprise a combination of methacrylic copolymers, as for example Eudragit® L 30 with Eudragit® FS 30D. Insoluble acrylic polymers, such as for example Eudragit® NE 30 D, Eudragit® RL30D and Eudragit® S30D may also be used alone, in combination or in mixture with pH-dependant polymers to form an efficient barrier coating. The amount of barrier coating may be between about 5% and about 60% of the weight of the enteric coating layered proton pump inhibitor microgranules. One example of a qualitative formula contemplated by the present invention that is based on Eudragit® E-PO contains enteric coated pellets equivalent to 20 mg to 80 mg of omeprazole/tablet, Eudragit® E-PO as barrier coating polymer, dibutylsebacate as plasticiser of the barrier coating, sodium laurylsulfate as an additive for dispersion of Eudragit® E-PO in aqueous solvent, and magnesium stearate as a lubricant and a mineral charge of coating film. The unit amount of such compound is calculated in order to obtain the different relative amount of Eudragit® E-PO in the barrier- and enteric-coated omeprazole pellets: —10% as the lowest quantity to provide a minimum delayed release time of approximately 10 minutes, —30% to provide an intermediate delayed release time of approximately 30 minutes, —60% as maximum value for a 60 minutes delayed release time.

Optionally, a barrier coating, if applied, may further comprise an opacifying agent, preferably titanium dioxide. An optional final polymeric coating, soluble in acidic condition, such as a hypromellose based film, may be applied over the methacrylic copolymer-based barrier coating.

According to another embodiment, the methacrylic copolymer-based barrier coating is obtained from a composition containing the following constituents: Eudragit® E-PO (methacrylic copolymer), dibutyl sebacate, sodium lauryl sulphate, magnesium stearate, titanium dioxide and purified water. Other enteric coated PPIs and formulations contemplated by the present invention are described in WO 03/007919 (PCT/SE02/01370), which is incorporated herein by reference in its entirety.

As with the modified release PPI compositions described above, the enteric coated compositions containing a PPI may be coated with a cholinergic agonist for immediate or modified release following oral administration, as described herein.

### Oral Liquids

Liquid oral pharmaceutical compositions of the present invention may be prepared by mixing omeprazole (Prilosec®) or other proton pump inhibitors with a solution including at least one cholinergic agonist and at least one buffering agent, with or without a parietal cell activator, as discussed above. The omeprazole or other proton pump inhibitor, which can be obtained from a capsule or tablet or obtained from the solution for parenteral administration, is mixed with a sodium bicarbonate solution to achieve a desired final omeprazole (or other PPI) concentration. As an example, the concentration of omeprazole in the solution can range from approximately 0.4 mg/ml to approximately 10 mg/ml or higher. For the omeprazole, the concentration in the solution may range from approximately 1 mg/ml to approximately 5.0 mg/ml or higher, with about 2.0 mg/ml to about 3.0 mg/ml being a standard concentration. For lansoprazole (Prevacid®), the concentration may range from about 0.3 mg/ml to about 10 mg/ml or higher, with a preferred concentration being between about 3 mg/ml and about 5 mg/ml. For bethanecol (Urecholine® and Duvoid®), the concentration may range from about 1 mg/ml to about 15 mg/ml or higher with the preferred concentration being between about 3 mg/ml and about 7 mg/ml. For pilocarpine (Pilopine® and Salogen®), the concentration may range from about 10 mg/ml to about 100 mg/ml or higher with the preferred concentration being between about 30 mg/ml and about 50 mg/ml.

Although sodium bicarbonate is a suitable buffering agent employed in the present invention to protect the PPI against acid degradation, many other weak and strong bases (and mixtures thereof) can be utilized. For the purposes of this application, “buffering agent” shall mean any pharmaceutically appropriate weak base or strong base (and mixtures thereof) that, when formulated or delivered with (e.g., before, during and/or after) the PPI, functions to substantially prevent or inhibit the acid degradation of the PPI by gastric acid sufficient to preserve the bioavailability of the PPI administered. The buffering agent is administered in an amount sufficient to substantially achieve the above functionality. Therefore, the buffering agent of the present invention, when in the presence of gastric acid, must only elevate the pH of the stomach sufficiently to achieve adequate bioavailability of the drug to effect therapeutic action.

Accordingly, examples of buffering agents include, but are not limited to, sodium bicarbonate, potassium bicarbonate, magnesium hydroxide, magnesium lactate, magnesium gluconate, aluminum hydroxide, aluminum hydroxide/sodium bicarbonate co-precipitate, a mixture of an amino acid and a buffer, a mixture of aluminum glycinate and a buffer, a mixture of an acid salt of an amino acid and a buffer, and a mixture of an alkali salt of an amino acid and a buffer. Additional buffering agents include sodium citrate, sodium tartarate, sodium acetate, sodium carbonate, sodium polyphosphate, potassium polyphosphate, sodium pyrophosphate, potassium pyrophosphate, disodium hydrogenphosphate, dipotassium hydrogenphosphate, trisodium phosphate, tripotassium phosphate, sodium acetate, potassium metaphosphate, magnesium oxide, magnesium hydroxide, magnesium carbonate, magnesium silicate, calcium acetate, calcium glycerophosphate, calcium chloride, calcium hydroxide, calcium lactate, calcium carbonate, calcium bicarbonate, and other calcium salts.

The pharmaceutically acceptable carrier of the oral liquid preferably comprises a bicarbonate salt of Group IA metal as buffering agent, and can be prepared by mixing the bicarbonate salt of the Group IA metal, preferably sodium bicarbonate, with water. The concentration of the bicarbonate salt of the Group IA metal in the composition generally ranges from about 5.0 percent to about 60.0 percent. The concentration of the bicarbonate salt of the Group IA metal may range from about 7.5 percent to about 10.0 percent. In one embodiment of the present invention, sodium bicarbonate is the preferred salt and is present in a concentration of about 8.4 percent.

The amount of sodium bicarbonate, e.g., about 8.4%, that may be used in the solution of the present invention is approximately 1 mEq (or mmole) sodium bicarbonate per 2 mg omeprazole, with a range of approximately 0.2 mEq (mmole) to 5 mEq (mmole) per 2 mg of omeprazole.

In another embodiment of the present invention, enterically-coated omeprazole particles are obtained from delayed release capsules (Prilosec.®). Alternatively, omeprazole powder can be used. The enterically coated omeprazole particles are mixed with a sodium bicarbonate (NaHCO₃) solution (about 8.4%), which dissolves the enteric coating and forms an omeprazole solution. The omeprazole solution has pharmacokinetic advantages over standard time-released omeprazole capsules, including: (a) more rapid drug absorbance time following administration for the omeprazole solution versus absorption following administration for the enteric-coated pellets; (b) the NaHCO₃ solution protects the omeprazole from acid degradation prior to absorption; (c) the NaHCO₃ acts as an antacid while the omeprazole is being absorbed; and (d) the solution can be administered through an existing indwelling tube without clogging, for example, nasogastric or other feeding tubes (jejunal or duodenal), including small bore needle catheter feeding tubes.

Additionally, various additives can be incorporated into the solution to enhance its stability, sterility and isotonicity. Further, antimicrobial preservatives, antioxidants, chelating agents, and additional buffers can be added, such as ambicin. Various antibacterial and antifungal agents such as, for example, parabens, chlorobutanol, phenol, sorbic acid, and the like may enhance prevention of the action of microorganisms.

It may also be desirable to include isotonic agents, for example, sugars, sodium chloride, and the like. Additionally, thickening agents such as methylcellulose are desirable to use in order to reduce the settling of the omeprazole or other PPIs and the cholinergic agonist from the suspension.

The liquid oral solution may further comprise flavoring agents (e.g., chocolate, root beer or watermelon) or other flavorings stable at pH 7 to 9, anti-foaming agents (e.g., simethicone 80 mg, Mylanta Gas Relief Formula®, Phazyme®) and parietal cell activators, as discussed above).

The present invention further includes a pharmaceutical composition that comprises a PPI, a cholinergic agonist and at least one buffering agent in a form convenient for storage, whereby when the composition is placed into an aqueous solution, the composition dissolves yielding a suspension suitable for enteral administration to a subject. The pharmaceutical composition may be in a solid form prior to dissolution or suspension in an aqueous solution.

The resultant PPI solution is believed to be stable at room temperature for several weeks and inhibits the growth of bacteria or fungi. By providing a pharmaceutical composition including a PPI, a cholinergic agonist and a buffer in a solid form, which can be later dissolved or suspended in a prescribed amount of aqueous solution to yield the desired concentration of a PPI, a cholinergic agonist and buffer, the cost of production, shipping, and storage are greatly reduced, and there is no need to refrigerate the solid form of the composition or the solution. Of course, it should be understood that an already prepared liquid PPI and/or cholinergic agonist may be substituted for the solid forms. Once mixed the resultant solution can then be used to provide dosages for a single patient over a course of time, or for several patients.

Examples of compositions and dosage forms that are contemplated by the present invention, include but are not limited to, those that are described in U.S. Pat. No. 6,645,988, U.S. Pat. No. 6,328,994, U.S. patent application Ser. No. 190,240 and Publication No. 20040006109, U.S. Patent Application No 260,132 and Publication No 20030144306, WO 2004/004719, WO 2004/004690, all of which are incorporated herein by reference in their entireties.

### Tablets and Other Solid Dosage Forms

As mentioned above, the formulations of the present invention can also be manufactured in concentrated forms, such as tablets, suspension tablets and effervescent tablets or powders, such that upon reaction with water or other diluent, the aqueous form of the present invention is produced for oral or enteral administration.

The present pharmaceutical tablets or other solid dosage forms disintegrate rapidly in aqueous media and form an aqueous solution of the PPI, cholinergic agonist and buffering agent with minimal shaking or agitation. Such tablets utilize commonly available materials and achieve these and other desirable objectives. The tablets or other solid dosage forms of this invention provide for precise dosing of a PPI and a cholinergic agonist that may be of low solubility in water. They are particularly useful for medicating children and the elderly and others in a way that is much more acceptable than swallowing or chewing a tablet. The tablets that are produced have low friability, making them easily transportable.

The term “suspension tablets” as used herein refers to compressed tablets which rapidly disintegrate after they are placed in water, and are readily dispersible to form a suspension containing a precise dosage of a PPI and a cholinergic agonist. The suspension tablets of this invention may comprise, in combination, a therapeutic amount of a PPI and a cholinergic agonist, a buffering agent, and a disintegrant. Examples of suspension tablets comprise
PPI—e.g., about 10 mg, 15 mg, 20 mg, 40 mg, 80 mg or 120 mg of omeprazole, esomeprazole, lansoprazole, rabeprazole and/or pantoprazole,
Cholinergic agonist—e.g., about 5 mg, 10 mg, 25 mg or 50 mg bethanecol and/or pilocarpine, and
Buffering Agent—e.g., about 1-80 mEq acid neutralizing capacity of sodium bicarbonate, calcium carbonate, calcium hydroxide, calcium lactate, calcium glycerophosphate, calcium acetate, magnesium carbonate, magnesium hydroxide, magnesium silicate, magnesium aluminate, aluminum hydroxide, aluminum magnesium hydroxide or magnesium oxide.

Of course, it should be understood that any formulation or dosage form of the present invention may comprise at the very least these combinations of a PPI and a cholinergic agonist.

Croscarmellose sodium is a known disintegrant for tablet formulations, and is available from FMC Corporation, Philadelphia, Pa. under the trademark Ac-Di-Sol.®. It is frequently blended in compressed tableting formulations either alone or in combination with microcrystalline cellulose to achieve rapid disintegration of the tablet.

Microcrystalline cellulose, alone or co-processed with other ingredients, is also a common additive for compressed tablets and is well known for its ability to improve compressibility of difficult to compress tablet materials. It is commercially available under the Avicel.®. Two different Avicel®. products are utilized, Avicel® PH which is microcrystalline cellulose, and Avicel® AC-815, a coprocessed spray dried residue of microcrystalline cellulose and a calcium, sodium alginate complex in which the calcium to sodium ratio is in the range of about 0.40:1 to about 2.5:1. While AC-815 is comprised of 85% microcrystalline cellulose (MCC) and 15% of a calcium, sodium alginate complex, for purposes of the present invention this ratio may be varied from about 75% MCC to 25% alginate up to about 95% MCC to 5% alginate. Depending on the particular formulation and active ingredient, these two components may be present in approximately equal amounts or in unequal amounts, and either may comprise from about 10% to about 50% by weight of the tablet.

The suspension tablet composition may, in addition to the ingredients described above, contain other ingredients often used in pharmaceutical tablets, including flavoring agents, sweetening agents, flow aids, lubricants or other common tablet adjuvants, as described above and as will be apparent to those skilled in the art. Other disintegrants, such as crospovidone, croscarmellose sodium and sodium starch glycolate may be employed as well as those mentioned before.

In addition to the suspension tablet, the solid formulation of the present invention can be in the form of a powder, a tablet, a capsule, or other suitable solid dosage form (e.g., a pelleted form or an effervescing tablet, troche or powder), which creates the inventive solution in the presence of diluent or upon ingestion. For example, the water in the stomach secretions or water that is used to swallow the solid dosage form can serve as the aqueous diluent.

Compressed tablets are solid dosage forms prepared by compacting a formulation containing an active ingredient and excipients selected to aid the processing and improve the properties of the product. The term “compressed tablet” generally refers to a plain, uncoated tablet for oral ingestion, prepared by a single compression or by pre-compaction tapping followed by a final compression.

Such solid forms can be manufactured as is well known in the art. Tablet forms can include, for example, one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmaceutically compatible carriers. The manufacturing processes may employ one, or a combination of, four established methods: (1) dry mixing; (2) direct compression; (3) milling; and (4) non-aqueous granulation. Lachman et al., The Theory and Practice of Industrial Pharmacy (1986), which is incorporated herein by reference in its entirety. Such tablets may also comprise film coatings, which preferably dissolve upon oral ingestion or upon contact with diluent, as discussed above.

Non-limiting examples of buffering agents which could be utilized in such tablets include sodium bicarbonate, alkali earth metal salts such as calcium carbonate, calcium hydroxide, calcium lactate, calcium glycerophosphate, calcium acetate, magnesium carbonate, magnesium hydroxide, magnesium silicate, magnesium aluminate, aluminum hydroxide or aluminum magnesium hydroxide. A particular alkali earth metal salt useful for making an antacid tablet is calcium carbonate.

An example of a low density alkali earth metal salt useful for making the granules according to the present invention is extra light calcium carbonate believed to be available from Specialty Minerals Inc., Adams, Me. The density of the extra light calcium carbonate, prior to being processed according to the present invention, is believed to be about 0.37 gm/ml.

The granules used to make the tablets according to one embodiment of the present invention are made by either spray drying or pre-compacting the raw materials. Prior to being processed into granules by either process, the density of the alkali earth metal salts useful in the present invention may range from about 0.3 gm/ml to about 0.55 gm/ml, preferably about 0.35 gm/ml to about 0.45 gm/ml, even more preferably about 0.37 gm/ml to about 0.42 gm/ml.

Additionally, the present invention can be manufactured by utilizing micronized compounds in place of the granules or powder. Micronization is the process by which solid drug particles are reduced in size. Since the dissolution rate is directly proportional to the surface area of the solid, and reducing the particle size increases the surface area, reducing the particle size increases the dissolution rate. Although micronization results in increased surface area possibly causing particle aggregation, which can negate the benefit of micronization and is an expensive manufacturing step, it does have the significant benefit of increasing the dissolution rate of relatively water insoluble drugs, such as a PPI.

The present invention also relates to administration kits to ease mixing and administration. A week or month's supply of powder or tablets, for example, can be packaged with a separate month's supply of diluent, and a re-usable plastic dosing cup. More specifically, the package could contain seven (7), fourteen (14) or thirty (30) suspension tablets containing about 10 mg, 15 mf, 20 mg, 40 mg or 80 mg of a PPI, about 5 mg, 10 mg, 25 mg or 50 mg of a cholinergic agonist, and ¼, ½ or 1 L or more of sodium bicarbonate about 8.4% solution, and a 30 ml dose cup. The user places the tablets in the empty dose cup, fills it to the 30 ml mark with the sodium bicarbonate, waits for it to dissolve (gentle stirring or agitation may be used), and then ingests the suspension. One skilled in the art will appreciate that such kits may contain many different variations of the above components. For example, if the tablets or powder are compounded to contain PPI, a cholinergic agonist and buffering agent, the diluent may be water, sodium bicarbonate, or other compatible diluent, and the dose cup can be larger than 30 ml in size. Also, such kits can be packaged in unit dose form, or as weekly, monthly, or yearly kits, etc.

The granulations used to form the tablets of the present invention may also be used to form rapidly disintegrating chewable tablets, lozenges, troches, or swallowable tablets.

Effervescent tablets and powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and tartaric acid. When the salts are added to water, the acids and the base react to liberate carbon dioxide gas, thereby causing effervescence.

The choice of ingredients for effervescent granules depends both upon the requirements of the manufacturing process and the necessity of making a preparation which dissolves readily in water. The two required ingredients are at least one acid and at least one base. The base releases carbon dioxide upon reaction with the acid. Examples of such acids include, but are not limited to, tartaric acid and citric acid. Preferably, the acid is a combination of both tartaric acid and citric acid. Examples of bases include, but are not limited to, sodium carbonate, potassium bicarbonate and sodium bicarbonate. Preferably, the base is sodium bicarbonate, and the effervescent combination has a pH of about 6.0 or higher.

Effervescent salts may include the following ingredients, which actually produce the effervescence: sodium bicarbonate, citric acid and tartaric acid. When added to water the acids and base react to liberate carbon dioxide, resulting in effervescence. It should be noted that any acid-base combination which results in the liberation of carbon dioxide could be used in place of the combination of sodium bicarbonate and citric and tartaric acids, as long as the ingredients were suitable for pharmaceutical use, and result in a pH of about 6.0 or higher.

It should be noted that it requires 3 molecules of NaHCO₃ (sodium bicarbonate) to neutralize 1 molecule of citric acid and 2 molecules of NaHCO₃ to neutralize 1 molecule of tartaric acid. One approximate ratio of ingredients is as follows citric acid:tartaric acid:sodium bicarbonate=1:2:3.44 (by weight). This ratio can be varied and continue to produce an effective release of carbon dioxide. For example, ratios of about 1:0:3 or 0:1:2 are also effective.

The method of preparation of the effervescent granules of the present invention employs three basic processes: wet and dry granulation, and fusion. The fusion method is used for the preparation of most commercial effervescent powders. It should be noted that although these methods are intended for the preparation of granules, the formulations of effervescent salts of the present invention could also be prepared as tablets, according to well known prior art technology for tablet preparation.

Wet granulation is the oldest method of granule preparation. The individual steps in the wet granulation process of tablet preparation include milling and sieving of the ingredients; dry powder mixing; wet massing; granulation; and final grinding.

Dry granulation involves compressing a powder mixture into a rough tablet or slug on a heavy-duty rotary tablet press. The slugs are then broken up into granular particles by a grinding operation, usually by passage through an oscillation granulator. The individual steps include mixing of the powders; compressing (slugging); and grinding (slug reduction or granulation). No wet binder or moisture is involved in any of the steps.

The fusion method is another method for preparing the granules of the present invention. In this method, the compressing (slugging) step of the dry granulation process is eliminated. Instead, the powders are heated in an oven or other suitable source of heat.

### Rapidly Disintegrating or Dissolving Nanoparticulate Compositions

The present invention also contemplates solid dose rapidly disintegrating or dissolving nanoparticulate compositions PPIs and cholinergic agonists. The rapidly disintegrating or dissolving solid oral dosage form has the advantage of combining rapid presentation of the PPIs and cholinergic agonists as a result of the rapid disintegration, and rapid dissolution of the PPIs and cholinergic agonists in the oral cavity as a result of the nanoparticulate size of the drugs.

It is believed that this combination of rapid disintegration and rapid dissolution reduces the delay in the onset of therapeutic action. Further, it is believed that the opportunity for buccal absorption of the PPIs and cholinergic agonists is enhanced with the present invention. Yet another advantage of nanoparticulate rapidly disintegrating or dissolving solid dose forms is that the use of nanoparticulate drug particles eliminates or minimizes the feeling of grittiness found with prior art fast melt formulations of poorly soluble drugs.

Rapidly disintegrating or dissolving dosage forms, also known as fast dissolve, fast or rapid melt, and quick disintegrating dosage forms, dissolve or disintegrate rapidly in the patient's mouth without chewing or the need for water within a short time frame. Because of their ease of administration, such compositions are particularly useful for the specific needs of pediatrics, geriatrics, and patients with dysphagia (difficulty swallowing). Rapidly dissolving dosage forms can be beneficial because of their ease of administration, convenience, and patient-friendly nature. It is estimated that a significant portion of the population finds it difficult to swallow tablets and hard gelatin capsules, particularly pediatric and geriatric patients. Rapidly disintegrating or dissolving dosage forms eliminate the need to swallow a tablet or capsule. Moreover, rapidly disintegrating or dissolving dosage forms do not require the addition of water or chewing.

Another advantage is believed to be that a reduction of the time lag between administration of a dose and the physical presentation of the active ingredient, e.g., PPIs and cholinergic agonists. This lag time is usually associated with the break up of the dosage form and the distribution of the active ingredients thereafter. A second advantage of these fast melt dosage forms is believed to be that the rapid presentation of the drug in the mouth upon administration may facilitate buccal absorption of the active ingredient, e.g., PPIs and cholinergic agonists, directly into the blood stream, thus reducing the first pass effect of the liver on the overall bioavailability of the PPIs and cholinergic agonists from a unit dose. This second advantage is also believed to be enhanced, as the nanoparticulate size of the PPIs and cholinergic agonists enables rapid dissolution in the oral cavity.

One example of a solid dose rapidly disintegrating formulation of the invention comprise a nanoparticulate PPI and a cholinergic agonist to be administered, having an effective average particle size prior to inclusion in the dosage form of less than about 2000 mm, at least one surface stabilizer adsorbed on the surface thereof, and at least one pharmaceutically acceptable water-soluble or water-dispersible excipient, which functions to rapidly disintegrate the matrix of the solid dose form upon contact with saliva, thereby presenting the nanoparticulate PPI and cholinergic agonist for absorption. The PPI and cholinergic agonist can be in a crystalline form, semi-crystalline form, amorphous form, or a combination thereof. Other examples of rapid or fast melt compositions contemplated by the present invention include, for example, those described in U.S. Pat. Nos. 6,375,982, 4,684,534, 4,609,543, 4,446,135, 4,327,077, 4,327,076, 5,464,632, 5,639,475, 5,709,886, 5,807,578, 5,807,577, 5,807,576, 5,635,210, 5,595,761, 5,587,180, 5,776,491 and 5,607,697, WO 98/46215, U.S. patent application Ser. No. 392383 and Publication No. 20030215502 and U.S. patent application Ser. No. 276400 and Publication No. 20040013613, all of which are incorporated herein by reference.

The effective average particle size of the PPI and cholinergic agonist prior to inclusion in the dosage form are believed to be less than about 1500 nm, less than about 1000 nm, less than about 600 nm, less than about 400 nm, less than about 300 nm, less than about 250 nm, less than about 100 nm, or less than about 50 nm. Nanoparticulate compositions were first described in the U.S. Pat. No. 5,145,684, which is incorporated herein by reference in its entirety herein throughout, the “'684 patent”).

A rapidly disintegrating nanoparticulate solid oral dosage form according to the invention has a disintegration time of less than about 3 minutes upon addition to an aqueous medium. More preferably, the fast melt nanoparticulate solid oral dosage form has a disintegration or dissolution time upon addition to an aqueous medium of less than about 2 minutes, less than about 90 seconds, less than about 60 seconds, less than about 45 seconds, less than about 30 seconds, less than about 20 seconds, less than about 15 seconds, less than about 10 seconds, or less than about 5 seconds.

The rapidly disintegrating or dissolving nanoparticulate dosage forms can have a relatively high degree of tensile strength. Tensile strength is determined by the hardness, size, and geometry of the solid dose. This is believed to be significant because if a solid does (i.e., a tablet) is too brittle, it will crumble or fragment. Such brittle tablets can also be difficult and expensive to package. Thus, the ideal rapidly disintegrating solid oral dose should have a degree of tensile strength to allow ease of packaging while also rapidly disintegrating upon administration. The rapidly disintegrating or dissolving solid dose nanoparticulate compositions can be formulated to mask the unpleasant taste of an active agent. Such taste masking can be accomplished, for example, by the addition of one or more sweet tasting excipients, such as those described above, by coating the PPI, cholinergic agonist and stabilizer with a sweet tasting excipient, and/or by coating a dosage form of PPI, cholinergic agonist, stabilizer, and excipients with a sweet tasting excipient.

The starting nanoparticulate composition (prior to formulation into a fast melt dosage form) comprises a PPI and a cholinergic agonist to be administered and at least one surface stabilizer adsorbed on the surface thereof.

The PPI and cholinergic agonist may be present in any amount which is sufficient to elicit a therapeutic effect and, where applicable, may be present either substantially in the form of one optically pure enantiomer or as a mixture, racemic or otherwise, of enantiomers, as discussed herein before.

Useful surface stabilizers, which are known in the art and described in the '684 patent, are believed to include those which physically adhere to the surface of the PPI and cholinergic agonist, but do not chemically bond to or interact with the PPI and cholinergic agonist. The surface stabilizer is adsorbed on the surface of the PPI and cholinergic agonist in an amount sufficient to maintain an effective average particle size of less than about 2000 nm for the active agent. Furthermore, the individually adsorbed molecules of the surface stabilizer should be essentially free of intermolecular cross-linkages. Two or more surface stabilizers can be employed in the compositions and methods of the invention.

Suitable surface stabilizers can preferably be selected from known organic and inorganic pharmaceutical excipients. Such excipients include various polymers, low molecular weight oligomers, natural products, and surfactants. Preferred surface stabilizers include nonionic and ionic surfactants.

Representative examples of surface stabilizers include gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens®. such as e.g. Tween 20® and Tween 80® (ICI Speciality Chemicals)); polyethylene glycols (e.g. Carbowaxs 3550.RTM. and 934® (Union Carbide)), polyoxyethylene stearates, colloidal silicon dioxide, phosphates, sodium dodecylsulfate, carboxymethylcellulose calcium, carboxymethylcellulose sodium, methylcellulose, hydroxyethylcellulose, hydroxypropylcellulose, hydroxypropylmethyl-cellulose phthalate, noncrystalline cellulose, magnesium aluminium silicate, triethanolamine, polyvinyl alcohol (PVA), polyvinylpyrrolidone (PVP), 4-(1,1,3,3-tetramethylbutyl)-phenol polymer with ethylene oxide and formaldehyde (also known as tyloxapol, superione, and triton), poloxamers (e.g., Pluronics F68® and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), dialkylesters of sodium sulfosuccinic acid (e.g., Aerosol OT®, which is a dioctyl ester of sodium sulfosuccinic acid (American Cyanamid)); Duponol P. RTM., which is a sodium lauryl sulfate (DuPont); Tritons X-200®, which is an alkyl aryl polyether sulfonate (Rohm and Haas); Crodestas F-100, which is a mixture of sucrose stearate and sucrose distearate (Croda Inc.); p-isononylphenoxypoly-(glycidol), also known as Olin-10G® or Surfactant 10-G® (Olin Chemicals, Stamford, Conn.); Crodestas SL-40® (Croda, Inc.); and SA9OHCO, which is C₁₈H₃₇CH.sub.2(CON(CH₃)—CH₂(CHOH)₄(CH₂OH)₂ (Eastman Kodak Co.); decanoyl-N-methylglucamide; n-decyl .beta.-D-glucopyranoside; n-decyl beta.-D-maltopyranoside; n-dodecyl beta.-D-glucopyranoside; n-dodecyl beta.-D-maltoside; heptanoyl-N-methylglucamide; n-heptyl-.beta.-D-glucopyranoside; n-heptyl beta.-D-thioglucoside; n-hexyl beta.-D-glucopyranoside; nonanoyl-N-methylglucamide; n-noyl beta.-D-glucopyranoside; octanoyl-N-methylglucamide; n-octyl-.beta.-D-glucopyranoside; octyl beta.-D-thioglucopyranoside; and the like.

Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), which specifically is incorporated herein by reference in its entirety.

As used herein, particle size is determined on the basis of the weight average particle size as measured by conventional particle size measuring techniques well known to those skilled in the art. Such techniques include, for example, sedimentation field flow fractionation, photon correlation spectroscopy, light scattering, and disk centrifugation.

By an “effective average particle size of less than about 2000 nm” it is meant that at least about 50% of the active agent particles have an average particle size of less than about 2000 nm when measured by the above techniques. Preferably, at least about 70% of the particles have an average particle size of less than the effective average, i.e., about 2000 nm, more preferably at least about 90% of the particles have an average particle size of less than the effective average. In one embodiment, the effective average particle size is less than about 1500 nm, less than about 1000 nm, less than about 600 nm, less than about 400 nm, less than about 300 nm, less than about 250 nm, less than about 100 nm, or less than about 50 nm.

The pharmaceutically acceptable water-soluble or water-dispersible excipient is typically a sugar, such as sucrose, maltose, lactose, glucose, or mannose; a sugar alcohol such as mannitol, sorbitol, xylitol, erythritol, lactitol, or maltitol; a starch or modified starch, such as corn starch, potato starch, or maize starch; a natural polymer or a synthetic derivative of a natural polymer, such as gelatin, carrageenin, an alginate, dextran, maltodextran, dextrates, dextrin, polydextrose, or tragacanth; a natural gum such as acacia, guar gum, or xanthan gum; a synthetic polymer, such as polyethylene glycol, polyvinylpyrrolidone, polyvinylalcohol, polyoxyethylene copolymers, polyoxypropylene copolymers, or polyethyleneoxide; or a mixture of any of these compounds. Other compounds believed to be useful include carbomers and cellulose-based polymers. The pharmaceutically acceptable water-soluble or water-dispersible excipient can be a direct compression or a non-direct compression disintegrant.

As with other pharmaceutical compositions according to the invention, these rapid melt compositions may also comprise one or more binding agents, filling agents, lubricating agents, suspending agents, sweeteners, flavoring agents, preservatives, buffers, wetting agents, disintegrants, effervescent agents, and other excipients, as described above and known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicifized microcrystalline cellulose (SMCC).

Suitable lubricants, including agents that act on the flowability of the powder to be compressed, are colloidal silicon dioxide, such as Aerosil.RTM. 200; talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet®, bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate, as discussed earlier herein. Suitable organic acids include, for example, citric, tartac, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the acid component of the effervescent couple may be present.

The relative amount of nanoparticulate composition in the rapidly disintegrating formulations of the invention can vary widely and can depend upon, for example, the PPI and cholinergic agonist selected for delivery, their melting points, water solubility, the surface tension of water solutions of the PPI and cholinergic agonist, etc. The PPI and cholinergic agonist or pharmaceutically acceptable salt thereof may be present in any amount which is sufficient to elicit a therapeutic effect and, where applicable, may be present either substantially in the form of one optically pure enantiomer or as a mixture, racemic or otherwise, of enantiomers.

It is believed that the nanoparticulate PPI/cholinergic agonist composition can be present in the rapidly disintegrating formulations in an amount of about 0.1% to about 99.9% (w/w), preferably about 5% to about 70% (w/w), and most preferably about 15% to about 40% (w/w), based on the total weight of the dry composition.

It is believed that the one or more pharmaceutically acceptable water-soluble or water-dispersible excipients can be present in an amount of about 99.9% to about 0.1% (w/w), preferably about 95% to about 30% (w/w), and most preferably about 85% to about 60% (w/w), by weight based on the total weight of the dry composition.

In another aspect, there is provided a method of preparing such rapidly disintegrating or dissolving nanoparticulate solid dose oral formulations. The method comprises: (1) forming a nanoparticulate composition comprising a PPI and a cholinergic agonist to be administered and at least one surface stabilizer; (2) adding one or more pharmaceutically acceptable water-soluble or water-dispersible excipients, and (3) forming a solid dose form of the composition for administration. It is thought that pharmaceutically acceptable excipients can also be added to the composition for administration. Methods of making nanoparticulate compositions, which can comprise mechanical grinding, precipitation, or any other suitable size reduction process, are known in the art and are described in, for example, the '684 patent.

Methods of making solid dose pharmaceutical formulations are known in the art, and such methods can be employed in the present invention. Exemplary rapidly disintegrating or dissolving solid dose formulations of the invention can be prepared by, for example, combining the one or more pharmaceutically acceptable water-soluble or water-dispersible excipients with a raw nanoparticulate dispersion obtained after size reduction of a PPI and a cholinergic agonist to be administered. The resultant composition can be formulated into tablets for oral administration. Alternatively, the nanoparticulate dispersion can be spray dried, followed by blending with one or more pharmaceutically acceptable water-soluble or water-dispersible excipients and tableting. The nanoparticulate dispersion and desired excipients can also be lyophilized to form a fast melt formulation, or the nanoparticulate dispersion can be granulated to form a powder, followed by tableting.

It is believed that solid dose forms of nanoparticulate dispersions can be prepared by drying the nanoparticulate formulation following size reduction. One drying method is spray drying. The spray drying process is used to obtain a nanoparticulate powder following the size reduction process used to transform the PPI and cholinergic agonist into nanoparticulate sized particles. Such a nanoparticulate powder may then be formulated into tablets for oral administration.

In an exemplary spray drying process, the nanoparticulate PPI/cholinergic agonist suspension is fed to an atomizer using a peristaltic pump and atomized into a fine spray of droplets. The spray is contacted with hot air in the drying chamber resulting in the evaporation of moisture from the droplets. The resulting spray is passed into a cyclone where the powder is separated and collected. The nanoparticulate dispersion may then be spray-dried in the presence or absence of excipients to give the spray-dried intermediate powder.

A rapidly disintegrating solid oral dosage form of the invention may also be prepared by lyophilizing a nanoparticulate dispersion of the PPI, cholinergic agonist and stabilizer. Suitable lyophilization conditions include, for example, those described in EP 0,363,365, U.S. Pat. No. 4,178,695, and U.S. Pat. No. 5,384,124, all of which are incorporated herein by reference in their entireties. Typically, the nanoparticulate dispersion is placed in a suitable vessel and frozen to a temperature of between about −5° C. to about −100° C. The frozen dispersion is then subjected to reduced pressure for a period of up to about 48 hours. The combination of parameters such as temperature, pressure, dispersion medium, and batch size will impact the time required for the lyophilization process. Under such conditions of reduced temperature and pressure, the frozen solvent is removed by sublimation yielding a solid, porous, rapidly disintegrating solid oral dosage form having the PPI and cholinergic agonist distributed throughout.

Alternatively, a rapidly disintegrating solid oral dosage form of the invention can be prepared by granulating in a fluidized bed an admixture comprising a nanoparticulate dispersion of the PPI and cholinergic agonist and at least one surface stabilizer with a solution of at least one pharmaceutically acceptable water-soluble or water-dispersible excipient, to form a granulate. This is followed by the granulate to form a solid oral dosage form.

Granulation of the nanoparticulate composition and at least one water-soluble or water-dispersible excipient can be accomplished using a fluid bed granulator or by using high shear granulation. Fluid bed drying can also be used in making a nanoparticulate dry powder for processing into a dosage formulation.

The rapidly disintegrating nanoparticulate solid formulations of the invention can be in the form of tablets for oral administration. Preparation of such tablets can be by pharmaceutical compression or molding techniques known in the art. The rapidly disintegrating nanoparticulate tablets as well as other tablets of the invention may take any appropriate shape, such as discoid, round, oval, oblong, cylindrical, triangular, hexagonal, and the like.

Powders for can be formulated into tablets by any method known in the art. Suitable methods include, but are not limited to, milling, fluid bed granulation, wet granulation, dry granulation, direct compression, spheronization, spray congealing, and spray-dying. Detailed descriptions of methods are provided in Remington's Pharmaceutical Sciences, Lippincott Williams & Wilkins, Philadelphia, 21st Edition, Chapter 45, pp. 888-928 (Mach Publishing Company, 2005), all of which is incorporated herein by reference in its entirety.

In an exemplary process, a rapidly disintegrating dosage form can be prepared by blending a nanoparticulate composition, comprising a PPI and a cholinergic agonist and at least one surface stabilizer, with at least one pharmaceutically acceptable water-soluble or water-dispersible excipient, and, optionally, other excipients to form a blend which is then directly compressed into tablets. For example, spray-died nanoparticulate powder can be blended with tablet excipients using a V-blenders® (blend Master Lab Blender, Patterson Kelley Co.) or high-shear mixer, followed by compression of the powder using, for example, an automated Carver press (Carver Laboratory Equipment), single station Korsch.RTM press, or a high-speed Fette® tablet press.

The tablets may be coated or uncoated. If coated they may be sugar-coated (to cover objectionable tastes or odors and to protect against oxidation) or film coated (a thin film of water soluble matter for similar purposes). Remington's Pharmaceutical Sciences, Lippincott Williams & Wilkins, Philadelphia, 21st Edition, Chapter 46, pp. 929-938 (Mach Publishing Company, 2005), all of which is incorporated herein by reference in its entirety.

The present invention provides a method of treating an animal, including a human, requiring the rapid availability of a PPI and a cholinergic agonist. The administered rapidly disintegrating or dissolving nanoparticulate compositions of the invention rapidly release an incorporated active agent resulting in fast onset of activity.

In general, the compositions of the invention will be administered orally to an animal subject in need thereof using a level of drug or active agent that is sufficient to provide the desired physiological effect. The animal subject, as discussed above, may be a domestic animal or pet, but preferably is a human subject.

As discussed herein above, the present invention also contemplates methods of doing business to treat or prevent a gastric acid disorder in an individual with a proton pump inhibitor, a cholinergic agonist and, optionally, an antacid, which include manufacture, distribution, repackaging, dispensing and/or instruction of these active ingredients individually or as unitary and single pharmaceutical compositions, in numerous dosage forms and strengths, so that once the proton pump inhibitor, the cholinergic agonist and, optionally, the antacid have been dispensed by a retailer, such as a pharmacy, to an individual, the individual may take the proton pump inhibitor, the cholinergic agonist and, optionally, an antacid at any time of the day or night without food or a meal or on an empty stomach, to treat or prevent the gastric acid disorder.

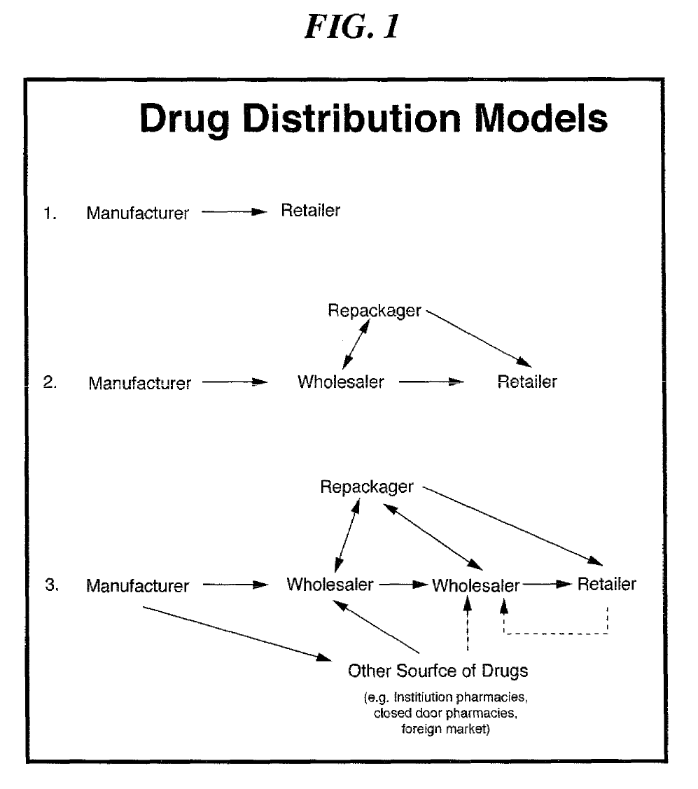

Turning now to FIG. 1, it depicts three exemplary models showing the manufacture and movement of drugs through a drug distribution system in accordance with the business methods to treat or prevent gastric acid disorders in accordance with the present invention. In the simplest situation, and in accordance with the business models of the present invention, a manufacturer, who manufactures a proton pump inhibitor, a cholinergic agonist and/or other drugs, namely, and antacid, sells or distributes either or both directly to a retailer. In a common business model as contemplated by the present invention, a manufacturer will sell or distribute the proton pump inhibitor, the cholinergic agonist and other drugs, such as an antacid, to a wholesaler, who then will sell or distribute them to a dispensing organization, such as retail chain stores, independent drug stores, and health care facilities. These drugs reach the ultimate consumer with a minimum number of transactions or physical shipments.

More complex models of distribution as contemplated by the present invention include those distribution models when a manufacturer offer price discounts the proton pump inhibitor, the cholinergic agonist and other drugs use in combination therewith, such as an antacid. Under this distribution model, a manufacturer will offer short-term sales for these individual drugs in order to reduce inventories or to meet quarterly sales targets. Large distributors, and especially secondary wholesalers, who are willing to risk substantial capital to acquire the discounted goods, will purchase these sale drugs in accordance with the business models of the present invention. These purchasers then turn the product over quickly by selling it to their networks of customers, which might include both larger and smaller distributors, and some drug dispensing organizations. In this model of drug dispersion, however, the sale drugs might change hands one or more times before reaching a drug dispenser (i.e., a retail pharmacy or a hospital).

It should be appreciated that the term “wholesaler”, as used herein generally refers to companies that engage in wholesale purchasing, distributing and reselling of pharmaceutical products. More specifically, the term Wholesale drug distributor shall mean any person or entity located in this state and engaged in wholesale drug distribution, including manufacturers, repackers, own-label distributors, jobbers, private-label distributors, brokers, warehouses including manufacturer and distributor warehouses, chain drug warehouses, and wholesale drug warehouses, independent wholesale drug traders, and retail pharmacies that conduct wholesale drug distribution.

While wholesalers also perform distribution functions, their activities do not always involve distribution in the sense of moving products closer to their eventual point of consumption. For example, a wholesaler could purchase an entire lot of distressed product and resell it, in its entirety, to another company, without “distributing” the product to smaller companies. The term “distribution” or “distributing” as used herein will refer to the physical activity of distributing the drugs among customers, namely, distributors, retailers, wholesalers, drug source entities, or repackagers.

Drug wholesalers serve as middlemen between drug manufacturers and prescription drug dispensers (i.e., retail outlets and institutions). Wholesalers generally provide a cost-effective means for the purchase, delivery, and sale of prescription drugs. They may improve purchasing economies and lower manufacturer costs by reducing the number of small volume sales by drug manufacturers. They also relieve retailers and institutions from the burden of dealing with each individual manufacturer for drug purchases.

Today, drug wholesalers consist of what has become known as the big five full-line wholesalers, i.e., McKesson HBOC, Inc., Bergen Brunswig Drug Company, Cardinal Health, Inc., AmeriSource Corporation, and Bindley Western Drug Company. Notwithstanding, wholesalers are generally classified into several categories based on their size, breadth of coverage and activity, and principal function. Thus, there are also regional wholesalers, and numerous smaller sub-regional/specialty wholesalers. The big five wholesalers, however, account for about 90% or more of the primary wholesale market. In addition, there are many smaller wholesalers who may have full or partial product lines and who may or may not sell nationally or regionally. Some of these wholesalers are “secondary wholesalers” who purchase selected drug products from wholesalers and they resell to other wholesalers, including large wholesalers, as well as pharmacies. In other words, they take advantage of manufacturers' sales on drugs to purchase discounted drug products and then resell these products throughout the distribution chain.

There are many reasons why sales from one wholesaler to another may benefit consumers. These may include: 1) taking advantage of price discounts available on certain legitimate drug products, (e.g., when a manufacturer or wholesaler has a temporary overstock or purchases excessive product on speculation that the manufacturer will raise prices), 2) low volume transactions (e.g., involving drugs that are used only occasionally in special populations), 3) quick turnaround (e.g., permitting a wholesaler or pharmacy to meet a temporary and unexpected increase in demand for a drug), or 4) sale to a remote area (e.g., sales to a small rural community).

Typically, major wholesalers have sophisticated ordering systems that allow customers to place and confirm orders electronically and to determine the availability and prices of wholesalers' stock. Wholesalers' inventory management systems help customers minimize carrying costs while maintaining adequate supplies to meet patients' needs. In most cases, wholesalers can also provide products within 24 hours. In addition to the delivery of drugs, wholesalers also provide a broad range of value-added services to pharmaceutical manufacturers, dispensers, and other customers, such as pharmacy benefit management companies (PBMs), clinical research organizations (CROs), group purchasing organizations (GPOs), and integrated delivery networks (IDNs). The major supplemental services offered by wholesalers include the following:
Private label/Control label programs-Number of wholesalers offer packaging and labeling operations in accordance with current Good Manufacturing Practices (CGMPs). The services offered typically include package configuration and product label design, filling and capping, labeling, and printing of bar coded product identification stickers.
Voluntary and/or co-op advertising programs—The cooperative advertising program is one in which the wholesaler provides marketing materials (i.e., store displays, flyers, etc.) to and reimburses the retail pharmacy for part or all of the retail pharmacy's advertising expenditures on selected products purchased from the wholesaler.
Special handling services for vaccines, frozen products, and orphan drugs.
Generic source programs—The program enables a wholesaler to combine the purchase volumes of its customers and negotiate prices with generic manufacturers. This results in competitive pricing of generic pharmaceuticals for the customers of the wholesaler.
Pharmacy computer systems—The pharmacy computer system facilitates the processing of prescriptions, drug interactions monitoring and claims processing.
Third-party claims processing—The claims processing system, which is integrated into the pharmacy computer system, facilitates real-time review and adjudication of prescriptions by third-party payers (i.e., health insurance companies). The system allows the pharmacist to establish patient eligibility, perform prospective drug utilization review (DUR), and notify the patient of any formulary requirements or prior authorization restrictions.
Retail-zone pricing systems—The products are delivered to the retail pharmacy with price labels already affixed to the individual containers so that the products can be immediately shelved.
Point-of-sale (POS) systems—The information technology (IT) system allows pharmacies to manage their inventory and ensure drug pricing accuracy. Typically, the POS systems feature bar code scanning and electronic credit card processing capabilities, which promote faster checkout at the cash register. The system also tracks product movement, identifying best and worst sellers, and facilitates better utilization of product shelf space. The system can generate a multitude of customized business management reports, including hourly product sales, monthly profit trends, and various cashier activities.

It is therefore believed that manufacturers sell a portion of their output directly to dispensing organizations, such as large retail pharmacy chains or healthcare organizations. In the past decade, institutional consumers of pharmaceutical drugs, such as hospitals and retail pharmacy chains, as well as independent retail pharmacies, have significantly decreased the percentage of pharmaceuticals purchased directly from the manufacturer. For these institutions, the value-added services of the distributor are more valuable than the price savings from dealing directly with the manufacturer. Conversely, mail order and internet pharmacies are believed to have increased the volume of pharmaceuticals they purchase directly from manufacturers. Mail-order and internet pharmacies are often used to dispense maintenance drugs regularly used by patients over an extended period of time or elective drugs which are not covered by the third party plans. Some large dispensing companies, especially chain drug stores, perform self-warehousing wherein they assume the task of distribution itself. Instead of relying upon an outside distributor, these retailers buy directly from the manufacturer; store the drugs in one or more of their own warehouses; and deliver the drugs to their retail stores as needed. Retail chains with four or more stores (including chain drug stores, mass merchandisers, and food stores) have increased the percentage of drugs they now self-warehouse. Thus, retail chains self-warehouse a majority of purchases made either directly from manufacturers or through wholesalers.

The second model of drug distribution characterizes the movement of the large bulk of pharmaceutical products. Most drug shipments move from the drug manufacturer to several large wholesalers (i.e., the Big Five and regional wholesalers) and then on to dispensers (i.e., health care organizations, institutional pharmacies, retail pharmacies, etc.). For these drugs, the number of transactions and the times that the drug product is handled and physically moved is the minimum necessary to reach an eventual consumer. Specifically, perhaps 2 transactions (manufacturer to wholesale distributor to pharmacy chain or other dispenser) are made before the product is consumed.

Additional tiers of distribution exist for drugs that are shipped to some of the smaller drug dispensers. As has been noted, the Big Five and even regional wholesalers often have volume requirements that exclude some small dispensers from using their services. In the case of physicians' offices or small healthcare facilities, their demand for drugs is also somewhat limited and/or specialized so that they do not require the services of a full-line distributor. Thus, a small wholesaler might report that his customer base consists of several hundred physicians' offices, selected Federal health facilities, selected health care clinics, and miscellaneous other dispensers. For this case, the number of drug transactions made from manufacturer to dispenser might be three or four (full-line to regional to small sub-regional to perhaps smaller wholesaler).

Discounted drugs are sometimes sold in substantial volumes and, in order to absorb the supply, dispersed widely throughout the distribution network. In these cases, the number of transactions made before the drug product reaches a dispenser can be quite large. Discounted products are also often sold to secondary wholesalers, although the Big Five or regional wholesalers also participate in such sales. The secondary wholesalers are notable, however, for their willingness to absorb the risk of large purchases of discounted products. In addition, discounted drugs are sometimes sold in substantial volumes and, in order to absorb the supply, dispersed widely throughout the distribution network. In these cases, the number of transactions made before the drug product reaches a dispenser can be quite large. Discounted products are often sold to secondary wholesalers, although the Big Five or regional wholesalers also participate in such sales. The secondary wholesalers are notable, however, for their willingness to absorb the risk of large purchases of discounted products.

Representatives of secondary wholesalers described a considerably lengthy set of transactions for many of the drug products they handle. First, while manufacturers sell the bulk of their output to the Big Five wholesalers, they sometimes wish to sell additional products separately from these relationships. As noted earlier, manufacturers will often announce short-term sales of products for various reasons, such as to meet quarterly sales goals, or to reduce inventory before a price increase. Wholesalers might also hold drug sales to eliminate slow-moving inventory.

Such discounted drugs are then purchased by wholesalers, with many purchases by wholesalers other than the Big Five. In making these sometimes large purchases of sale merchandise, the wholesalers may incur a substantial capital investment and, less significantly, also use warehouse space to hold the drugs. Furthermore, many of these wholesalers do not have a normal or routine distribution channel that can absorb the discounted product quickly. The wholesalers are interested, therefore, in turning over products quickly and can do so by passing on a portion of the original discount to other wholesalers or drug purchasers. Thus, the original purchaser makes a large capital investment and attempts to recoup it as quickly as possible by selling portions of the sale product, at a still somewhat discounted price, to other wholesalers.

The second tier of wholesalers are believed to largely be in the same position as the original purchaser, although they are handling small volumes of sale products. Nevertheless, they may make relatively large capital investments and wish to turn over the discounted product as quickly as possible. In this fashion, the sale product is distributed rapidly and with broad dispersion, throughout the drug distribution industry. This second tier might include any drug wholesale organization, including the Big Five, regional, mail order, or other organizations.

The breadth of dispersion is indicated by the number of transactions that might occur before the sale product reaches the dispenser. According to one secondary wholesaler, it is believed to be not uncommon for his company to be among the third tier of distributors to purchase some of the sale product. Further, this executive judged it likely that the product would trade hands two or three more times before reaching the eventual drug dispenser. Thus, from 5 to perhaps 7 transactions involving the sale product are commonplace.

The business methods of the present invention also include manufacturing the proton pump inhibitors, cholinergic agonists and other drugs, such as and antacid, for optional use in combination therewith in accordance with this invention. By the term “manufacture(s)” or “manufacturing”, these terms as used herein generally refer to a process of preparing, propagating, formulating, synthesizing, isolating, purifying, making, mixing, blending, compounding, processing, packaging, repackaging, labeling, relabeling, testing, or quality control of the drug or drug product.

The business methods of the present invention also include repackaging the proton pump inhibitors, cholinergic agonists and other drugs, such as and antacid, for optional use in combination therewith in accordance with this invention. The terms “repackage(s)”, “repackaged” and “repackaging” are used interchangeably herein and mean the act of repackaging, relabeling and/or private labeling a quantity of the drug or drug product from a manufacturer's original container into an unit dose packaging or a multiple dose container for further distribution or later resale or relabeling and/or private labeling the manufacturer's original container housing a quantity of the drug or drug product for further distribution or later resale.

The term “retailer(s)”, is broadly used herein and means any person, entity, business, dealer and the like that is carrying on the retail business of sale of drugs to customers, such as pharmacies, department stores, grocery stores, super-markets, hyper-markets and the like. The term “pharmacy(ies)”, is used herein broadly and is intended to refer to any domestic or foreign pharmacy and includes community, independent, chain, mail-order, internet and retail pharmacies. The term “institutional pharmacy(ies)” is also broadly used herein and means any hospital, convalescent home, nursing home, extended care facility, mental institution, rehabilitation center, retardation center, correctional facility, hospice, out-patient surgery facility and the like. By the term, “closed-door pharmacy(ies)”, it is meant to refer to those pharmacies that buy drugs through intermediary purchasing groups at greatly reduced prices and are generally, but not necessarily, pharmacies that provide drugs to people in institutional pharmacies, such as nursing homes and hospitals.

Thus, and in accordance with the present invention, there can be one or more distributors, wholesalers, drug source entities or repackagers, who handle the drug(s) or drug products of the present invention one or more times before they reach the retailer, as shown in FIG. 1.

The following examples are provided solely to illustrate representative embodiments of the present invention. Accordingly, it should be understood, that the invention is not to be limited to the specific conditions or details described in these or any other examples discussed herein or construed as limiting the scope of the present invention in any way. Throughout this specification and these examples, any and all references to publicly available documents are specifically incorporated herein by reference in their entireties, even when not specifically so stated.

### Chewable and Bioerodable Tablets

The term “bioerodable” as used herein means that the component, carrier, or formulation erodes, over time, in biological media such as bodily fluids and anatomical structures comprising or bathed by body fluids. Examples of bodily fluids include blood, plasma, saliva, tears, lymph, urine, etc. Examples of anatomical structures comprising or bathed by bodily fluids include the oral cavity, the nasal cavity, the genitourinary tract, the respiratory tract, the gastrointestinal tract, etc. Such erosion in bodily fluids may be due to factors such as dissolution, dispersion, friction, gravity, etc. The terms water-erodable and bioerodible are used interchangeably.

In one example of a chewable tablet in accordance with the present invention is a multi-layered oral pharmaceutical dosage form that comprises at least one proton pump inhibitor layer, at least one cholinergic agonist layer and at least one antacid layer. In one embodiment, the antacid layer and the entire dosage form is free of sodium bicarbonate and any other effervescent materials. Also, the entire dosage form is free of any enteric coatings. The dosage form may be in the form of a multi-layered compressed tablet or caplet or a multi-layered filled gelatin capsule. In another embodiment, the dosage form is chewable or rapidly disintegrating.

The pharmaceutically acceptable excipients such as binders, fillers, lubricants, glidants, disintegrants and taste masking agents which are combined with the proton pump inhibitor, cholinergic agonist and antacid are described herein above and commonly known in the art. Many of these pharmaceutically acceptable excipients are described in the Remington, the Science and Practice of Pharmacy, Lippincott Williams & Wilkins, Philadelphia, 20th Edition, Chapter 45, pp. 858-893 (Mack Publishing Company, 2003) and the United States Pharmacopeia (USP 26), which are incorporated herein by reference in their entireties.

If a binder is used in the present invention, it may be any pharmaceutically acceptable non-toxic pharmaceutically acceptable binder, as described hereinbefore. The binder may be a water-soluble polymer of the group consisting of polyvinyl alcohol, polyvinylpyrrolidone, methylcellulose, hydroxypropyl cellulose, hydroxymethyl cellulose, hydroxy propyl methyl cellulose, hydroxyethyl methyl cellulose, gelatin, pectin, carrageenan, compressible sugars, sodium carboxymethyl cellulose, liquid glucose, alginates and gums and the like. The binder may also be a water insoluble binder such as ethylcellulose, acrylic or methacrylic polymers or copolymers, tragacanth, starch and pregelatinized starch and the like.

When a filler is used in the present invention, it may be any pharmaceutically acceptable, non-toxic pharmaceutically acceptable binder, as described hereinbefore. Common fillers are sugars such as lactose, dextrose, sucrose, maltose, mannitol, sorbitol, dibasic calcium phosphate and various starches or microcrystalline cellulose.

Examples of disintegrants that can be used in the present invention are those as described and above, including cornstarch, croscarmelose sodium, crospovidone (polyplasdone XL-10), sodium starch glycolate (EXPLOTAB or PRIMOJEL) or any combination of the foregoing. A preferred disintegrant is believed to be crospovidone or sodium starch glycolate.

Taste masking agents should be included for the chewable and rapidly disintegrating dosage forms of the present invention and include artificial sweeteners such as aspartame, saccharin, dipotassium glycyrrhizinate, stevia, thaumatin, as discussed above, and flavorants such as citric acid, peppermint oil, wintergreen oil, menthol, lemon, lime, orange grape, cherry and vanilla extract, also as discussed above. Additional taste masking agents are described in U.S. Pat. No. 6,027,746 and Vol. 1, pages 306-309 of Pharmaceutical Dosage Forms (Tablets) by Lieberman and Lachman, © 1982, which are incorporated herein by reference in their entireties. In one embodiment of the present invention, the taste masking agent comprises a mixture of artificial sweeteners and flavorants such as aspartame and berry or grape extract.

An alkaline agent may be necessary to stabilize the proton pump inhibitor during manufacture and storage of these or other dosage forms of the present invention. The alkaline agent can be any type of alkaline agent such as amino acids such as lysine, arginine, omitine, histidine, organic buffering compounds such as tromethamine, N-amino sugars, such as meglumine, eglumine, glucosamine, heterocyclic amine derivatives such as piperazine, alkali salts of citric acid, tartaric acid, caproic acid or fatty acids, alkali metal phosphates, silicates, hydroxides or carbonates, organic amines such as ethylamine, alkaline ammonium salts and combinations of the foregoing. Additional examples of alkaline agents can be found in U.S. Pat. No. 6,013,281, which is incorporated herein by reference in its entirety. The preferred alkaline agents are amino acids such as arginine, lysine or meglumine.

The present invention may also comprise, as discussed above, conventional processing aids such as tablet lubricants (magnesium stearate, sodium stearate), glidants (colloidal silicon dioxide) and wetting agents or stabilizers and surfactants (sodium lauryl sulfate, polysorbates). The processing aids are generally added to the dosage formulation in small amounts (less than 5 weight percent of the total weight of the formulation) and do not materially affect the properties of the final dosage formulation. Some of the aforementioned excipients can perform more than one function in the formulation. For example, sucrose and lactose can serve as fillers and sweeteners and microcrystalline cellulose can serve as a filler and a disintegrant depending upon the amount and manner used. The multi-function excipients are known to those skilled in the art.

The combination may comprise components in many different dosage strengths. Some examples of dosage strengths are herein provided, the strengths are meant by way of example and are in no way intended to be limiting or encompassing.

The antacid should be sufficient to neutralize the acid in the stomach and allow the proton pump inhibitors to be absorbed in the stomach and/or pass through the stomach relatively intact. Proton pump inhibitors are acid labile and therefore the antacid in the composition must be present in a sufficient amount to neutralize the acid in the stomach in order to protect the combination product. The neutralization of the stomach acid will also provide the added benefit of immediate relief for a patient until the proton pump inhibitor can begin to inhibit acid secretion.

As mentioned above the present invention can be prepared by any number of conventional dosage forming techniques known to those skilled in the art such as granulation, direct compression and/or capsule filling. In one embodiment of the present invention, the antacid, the cholinergic agonist and the proton pump inhibitor are separately granulated. The antacid granules will comprise at least the antacid and a binder. The cholinergic agonist granules will comprise at least the cholinergic agonist and a binder. The proton pump inhibitor granules will comprise at least the proton pump inhibitor, a binder and an alkaline agent, preferably an alkaline amino acid. The granules may also comprise a filler, a disintegrant, a glidant, a lubricant and a taste masking agent, as discussed previously. The granules can be made by wet or dry techniques commonly employed in the art. Slugging may also be employed to make the granules. In one embodiment of the present invention, the antacid granules, cholinergic agonist granules and the proton pump inhibitor granules are prepared by a wet granulation technique. In another embodiment, the antacid granules, cholinergic agonist granules and the proton pump inhibitor granules are made by dry granulation techniques, such as roller compaction. In a further embodiment, the antacid granules are made by roller compaction, the cholinergic agonist granules are made by wet granulation or by roller compaction, and the proton pump inhibitor granules are made by wet granulation.

Because many of the proton pump inhibitors such as omeprazole elicit a bitter taste that is difficult to mask simply by the addition of sweeteners and flavoring agents, it may be necessary to coat or encapsulate the proton pump inhibitors with a film-forming polymer or a wax material, as described herein before, especially if the dosage form of the present invention will be chewable or rapidly dissolving in the mouth. One particularly acceptable approach for coating the proton pump inhibitor involves melt granulation.

In melt granulation, a congealable solid, preferably a wax such as glyceryl monostearate or castor oil, is employed to coat or embed the proton pump inhibitor or cholinergic agonist and thereby mask the bitter taste. The congealable solid must be non-toxic, stable with a low melting point and no interaction with the drug that will affect its bioavailability. The congealable solid is heated until it melts. The cholinergic agonist or proton pump inhibitor and other excipients such as an alkaline material and a plasticizer are dispersed into melted material preferably by using a high-shear granulator with a temperature bath or control element that will prevent the congealable material from prematurely cooling and solidifying. After the cholinergic agonist or proton pump and other excipients are dispersed in the melted congealable material the dispersion is allowed to cool and coated cholinergic agonist or proton pump inhibitor granules are formed.

If a polymer coating of the proton pump inhibitor or cholinergic agonist is selected, a fluidized bed or pan coater may be used to apply a polymer dispersion or solution onto a mixture of the proton pump inhibitor or cholinergic agonist and selected excipients such as an alkaline material. The polymers used to coat and taste mask the proton pump inhibitor or cholinergic agonist can be film forming water soluble or water insoluble polymers such as ethylcellulose, hydroxypropyl methylcellulose, hydroxypropyl cellulose or combinations of the foregoing. It is also possible to use a combination of congealable material and polymer to coat the proton pump inhibitor or cholinergic agonist. In one embodiment of the present invention, no acidic film forming polymers such as enteric polymer should be used. In an alternate embodiment enteric film forming polymers can be used.

Once the antacid granules, the cholinergic agonist granules and the proton pump inhibitor granules are prepared, they are then further mixed with additional excipients such as a taste masking agent, a glidant and a lubricant to form an antacid laying mixture, a cholinergic agonist layering mixture and a proton pump layering mixture. The layering mixtures may also be mixed with additional fillers, binders and disintegrants. Depending upon the ingredients selected for the dosage formulation, the prior formation of antacid granules, cholinergic agonist granules and proton pump inhibitor granules may not be necessary. If the materials selected for use in the antacid layering mixture, the cholinergic agonist layering mixture and the proton pump inhibitor layering mixture allow sufficient flow of the mixtures into a tablet die or capsule without the need for a granulation step, the mixtures can be fed directly into a tablet press or capsule filing machine for the formation of the final dosage form.

After the antacid layering mixture, the cholinergic agonist layering mixture and the proton pump inhibitor layering mixture have been prepared, with or with out the granulation step, the layering mixtures are then formed into the final dosage form. In one embodiment, a predetermined amount of cholinergic agonist layering mixture is fed into a tablet press to form the cholinergic agonist layer, a predetermined amount of the proton pump inhibitor layering mixture is fed into the tablet press to form the proton pump inhibitor layer and then the antacid layering mixture is fed into the tablet press to form the antacid layer of the multi-layer tablet. It should be appreciated that the order in which the proton pump inhibitor layer, the cholinergic agonist layer and the antacid layer are fed into the tablet press can be reversed or changed. Additional antacid layers, cholinergic agonist layers and proton pump inhibitor layers can also be fed into the tablet press. In one embodiment, the proton pump inhibitor layer is sandwiched between two antacid layers that contain the same or different antacids and two cholinergic agonist layers that contain the same or different cholinergic agonist.

If a capsule is the final dosage form, a predetermined amount of cholinergic agonist layering mixture is fed into one third of a capsule. A predetermined amount of the proton pump inhibiting layering mixture is then fed into another third of a capsule. Once the proton pump inhibiting layering mixture is in the capsule, a predetermined amount of the antacid layering mixture is added to the capsule and forms an antacid layer on top of the proton pump inhibitor layer, which is on top of the cholinergic agonist layer. Once both the proton pump inhibitor layer and the antacid layer are in the capsule, the capsule is sealed. Again, the order in which the proton pump inhibitor, cholinergic and antacid layers are placed in the capsule can be reversed or changed, as well as the inclusion of additional layers. In an alternate capsule embodiment, a predetermined amount of a proton pump inhibitor layering mixture is placed into a small capsule and sealed. The small capsule is then placed into a larger capsule with predetermined amounts of an antacid layering mixture and a cholinergic antagonist layering mixture and the larger capsule scaled to form the multi-layer dosage formulation of the present invention. Again, the order in which the proton pump inhibitor, cholinergic agonist and antacid layering mixtures are placed into the capsules can be reversed without departing from the scope of the present invention.

If a capsule is used in the final dosage form and the dosage form is designed to rapidly dissolve in the mouth, the capsule selected should be rapidly disintegrating. Such rapidly disintegrating capsules are commercially available from CAPSUGEL of Morris Plains, N.J. under the trade name NPcaps®.

The phrase “predetermined amount” used above means an amount of layering mixture that is calculated to provide a therapeutic amount of the proton pump inhibitor (e.g., about 5-80 mg), a therapeutic amount of a cholinergic agonist (e.g., about 5 mg to 50 mg) and/or a therapeutic amount of antacid activity (e.g., about 1-80 mEq of acid neutralizing capacity).

If granules are employed in the present invention, the granules may comprise the following:

Proton Pump Inhibitor Granules: Proton Pump Inhibitor about 5-60%, Alkaline Material about 5-60%, Filler about 10-90%, Binder about 0-50%, Disintegrant about 0-60%, Lubricant about 0-10%, Glidant about 0-10%;

Antacid Granules Antacid about 30-99%, Binder about 0.1-40%, Filler about 0-60%, Disintegrant 0-60%;

Cholinergic Agonist Granules: Cholinergic Agonist about 5-60%, Binder about 0.1-40%, Filler about 10-90%, Disintegrant about 0-60%, Lubricant about 0-10%, Glidant about 0-10%.

The granules are further processed into distinct layering mixtures for tableting or capsules as follows.

Proton pump inhibitor layering mixture comprises proton pump inhibitor granules about 40-99%, about 50-95% taste masking agent, about 0-40% lubricant, about 0-10% glidant. The taste masking agent preferably is a combination of a 0.1-99% sweetener and 0.1 to 99% favoring agent.

Cholinergic agonist layering mixture comprises about 40-99% cholinergic agonist granules, about 0-40% taste masking agent, about 0-40% lubricant, about 0-10% glidant. The taste masking agent preferably is a combination of about 0.1-99% sweetener and about 0.1-99% favoring agent.

Antacid layering mixture comprises about 40-99% antacid granules, about 0-40% taste masking agent, about 0-10% lubricant, about 0-10% glidant. The taste masking agent preferably is a combination of about 0.1-99% sweetener and about 0.1-99% favoring agent.

The layering mixtures are individually processed on a tablet press to produce a multi-layered (i.e., trilayer) chewable tablet, or rapidly disintegrating tablets. The layering mixtures may also be individually processed into capsules or tablet-filled capsules. Whether the final dosage form is a tablet or capsule, the antacid layer should comprise 40-95% of the final tablet weight, preferably, 50-85% and most preferably 60-80% and the proton pump inhibitor layer and the cholinergic agonist layer should comprises 5-60% of the final tablet weight, preferably, 15-50% and most preferably 20-40%. As mentioned above, the layering mixtures may not need the prior formation of granules. If the granules are not employed, the layering mixtures may comprise above mentioned granule excipients in similar amounts only in a non-granule form.

In another example of an oral chewable pharmaceutical tablet in accordance with the present invention, a multiparticulate tablet, which disintegrates in the mouth and provides good mouth feel is disclosed. In accordance with this embodiment, the multiparticulate tablet contains: i) a proton pump inhibitor in the form of enteric coated microgranules, which enteric coated granules are over-coated with at least one barrier coating, such as (for instance) a methacrylic copolymer-based protective film; ii) at least one cholinergic agonist in the form of granules; at least one antacid in the form of granules, for instance based on CaCO₃ and/or Mg(OH)₂ and/or Al(OH)₃; and, iii) a mixture of excipients comprising for example, a disintegrating agent, a diluent, a lubricant, and optionally a swelling agent, a permeabilizing agent, sweeteners, flavorings and colors, as discussed above. Furthermore, the present invention is directed to processes for the manufacture of the tablet and its use in the treatment of gastrointestinal disorders.

It is believed that such multiparticulate tablets will maintain stability of the enteric coating film within the oral disintegratable tablet containing the antacid agent together with an enteric coated proton pump inhibitor microgranules and the cholinergic agonist granules during storage and use. To this end, a barrier coating, as described herein above, is applied to protect the enteric coating from dissolution and/or disintegration in the mouth and/or stomach before the PPI microgranules are transported into the small intestine. It is further believed that the tablet according to the present invention will show satisfactory enteric properties of enteric microgranules, and provide a quick dissolution of the cholinergic agonist in the buccal cavity and/or gastrointestinal tract, and the proton pump inhibitor in the small intestine.

As further indicated herein above, the PPIs may be used in the form of its racemate or a single enantiomer, in the non-salt form or in the form of an alkaline salt of the racemate or one of its single enantiomers.

According to one embodiment, the PPIs are prepared, as discussed above under the section entitled Enteric Coated PPI Compositions, in the form of enteric coating layered microgranules consisting of a core comprising the said agent optionally in mixture with an alkaline reacting compound. The core is covered by a separating layer and an enteric coating layer, and the enteric coated microgranules being over-coated with the barrier coating, such as for instance a methacrylic copolymer-based film.

With respect to the antacid, while any suitable antacid is contemplated, as discussed above, the classical powder grades of antacid agents show bad properties, and bad organoleptic properties especially regarding mouth feeling and taste. Therefore, the antacid agent is preferably used in the form of granules. The antacid may, for example, be obtained by dry granulation of CaCO₃ and/or Mg(OH)₂ and/or Al(OH)₃ with mannitol, followed by wet granulation using a solution of xylitol and/or sorbitol. Antacid granules may optionally include a disintegrating agent and/or a permeabilization agent.

The antacid granules according to the invention present particle size distribution between about 150 μm and 710 μM, preferably between 355 μM and 710 μM, such that at least 50%, preferably at least 70% of the granules have a particle size ranging between about μM 150 and 710 μM and less than 20% of the granules have a particle size less than 150 μM. The particle sizes are measured according to conventional methods, preferably by sieving.

The tablets of the present invention also comprise a mixture of excipients as discussed previously herein. For example, a diluent agent may be selected from water-soluble and/or water-insoluble tableting filler. The water-soluble diluent agent is constituted from a polyol of less than 13 carbon atoms, in the form of directly compressible material (the mean particle size being between 100 and 500 microns), in the form of a powder (the mean particle size being less than 100 microns) or a mixture thereof. The polyol is preferably chosen from the group comprising of mannitol, xylitol, sorbitol and maltitol. The water-insoluble diluent agent is a cellulosic derivative preferably microcrystalline cellulose. A disintegrating agent is chosen from the group consisting of crosslinked sodium carboxymethylcellulose, crospovidone and their mixtures. A part of the disintegrating agent is used for the preparation of antacid granules. The lubricant agent is chosen from the group consisting of magnesium stearate, sodium stearylfumarate, stearic acid, Macrogol 6000 and their mixtures. While one part of the lubricant is used as an internal solid lubricant, another part is sprayed over the outer surface of the tablet. A swelling agent is chosen from the group consisting of starch, modified starch or microcrystalline cellulose.

The permeabilizing agent is chosen from the group consisting of silica having a high affinity with aqueous solvents, such as SYLOID®, maltodextrines, beta-cyclodextrines and their mixtures. The permeabilizing agent enables creation of a hydrophilic network that enhances the penetration of the saliva and the disintegration of the tablet. A part of permeabilizing agent is advantageously used for the preparation of antacid granules. A sweetener can be chosen in the group consisting of aspartame, potassium acesulfame, sodium saccharinate, dihydrochalcone neohesperidine and their mixtures. A flavoring is advantageously chosen to give a combination of fast onset and long-lasting sweet taste and get a “round feeling” in the mouth with different textures or additives. It should be noted that a combination of potassium acesulfame with aspartame may be preferred as a sweetener agent in such formulations. Cooling agents may also be added in order to improve the mouth feeling and provide a synergy with flavors and sweetness.

According to a preferred embodiment, a microparticulate tablet has the following composition:

i) barrier coated PPI microgranules, e.g., omeprazole (a) enteric coating layered omeprazole magnesium microgranules, (b) Eudragit® E PO (methacrylic copolymer), (c) dibutyl sebacate, (d) sodium lauryl sulphate, (e) magnesium stearate, (f) purified water, and (g) optionally titanium dioxide, hypromellose, talcum;

ii) cholinergic granules, e.g., bethanechol or pilocarpine;

iii) antacid granules (a) CaCO₃, (b) Mg(OH)₂, (c) Mannitol, (d) Sorbitol, (e) Purified water, and (D optionally crospovidone and silica; and

iv) excipients for formulation of the tablet (a) mannitol or microcrystalline cellulose, (b) crospovidone or croscarmellose, (c) aspartame, (d) flavorings, (e) silica, (f) magnesium stearate.

Water is used as a solvent and removed during the coating and the granulation processes.

In one aspect of the invention, the tablet of the invention is a bioerodible multiparticulate tablet that disintegrates in contact with the saliva, without chewing, in less than 60 seconds, preferably in less than 40 seconds.

According to one embodiment, the bioerodible tablet has the following composition:

i) barrier coated PPI microgranules, e.g. omeprazole (a) enteric coating layered PPI, e.g., omeprazole magnesium microgranules (b) Eudragit® EPO (methacrylic copolymer) (c) dibutyl sebacate (d) sodium lauryl sulphate, (e) magnesium stearate (f) purified water, and (c) optionally titanium dioxide, hypromellose, talcum,

ii) cholinergic granules, e.g., bethanechol or pilocarpine,

iii) Antacid granules (a) CaCO₃, (b) Mg(OH)₂, (c) mannitol, (d) sorbitol, (e) purified water, and (f) optionally crospovidone and silica,

iv) excipients for formulation of the tablet (a) mannitol, (b) crospovidone, (d) aspartame, (e) flavorings, (f) silica, (g) magnesium stearate and (h) optionally cooling agents.

According to another embodiment, the bioerodible tablet has the following composition:

i) barrier coated PPI microgranules, e.g., omeprazole (a) enteric coating layered PPI, e.g., omeprazole magnesium microgranules, (b) Eudragit® EPO (methacrylic copolymer), (c) dibutyl sebacate, (d) sodium lauryl sulphate, (e) magnesium stearate, (i) purified water, and (g) optionally titanium dioxide, hypromellose and talcum,

ii) cholinergic granules, e.g., bethanechol or pilocarpine,

iii) antacid granules (a) CaCO₃, (b) Mg(OH)₂, (c) mannitol, (d) sorbitol, (e) purified water, and (f) optionally crospovidone and silica, and

iv) excipients for formulation of the tablet (a) microcrystalline cellulose, (b) crospovidone, (c) aspartame, (d) flavorings, (e) silica, (f) magnesium stearate, and (g) optionally cooling agents.

In another aspect of the invention, the invention is a chewable multiparticulate tablet.

According to another embodiment, a chewable tablet has the following composition:

i) barrier coated PPI, e.g., omeprazole microgranules (a) enteric coating layered PPI, e.g., omeprazole magnesium microgranules, (b) Eudragit® E PO (methacrylic copolymer), (c) dibutyl sebacate, (d) sodium lauryl sulphate, (e) magnesium stearate, (f) purified water, and (g) optionally titanium dioxide, hypromellose and talcum,

ii) cholinergic granules, e.g., bethanechol or pilocarpine,

iii) antacid granules (a) CaCO₃, b) Mg (OH)₂, (e) mannitol, (d) sorbitol, (e) purified water, and (f) optionally crospovidone and silica, and

iv) excipients for formulation of the tablet (a) microcrystalline cellulose, (b) croscarmellose, (c) aspartame, (d) flavorings, (e) silica, (f) magnesium stearate, and (g) optionally cooling agents.

According to another embodiment, the tablet of the invention, either bioerodible or chewable, has the following composition:

i) barrier coated omeprazole microgranules (a) enteric coating layered omeprazole microgranules ca 100 mg/equivalent to about 20 mg of omeprazole or an amount equivalent to about 80 mg of omeprazole, (b) Eudragit® E PO about 10-60 mg, (c) dibutyl sebacate about 1-10 mg, (d) sodium lauryl sulphate about 0.5-5 mg, (e) magnesium stearate about 2. 5-15 mg, (f) purified water,

ii) cholinergic granules, e.g., bethanechol or pilocarpine (a) bethanecol about 5 mg-50 mg or (b) pilocarpine about 5 mg to about 7.5 mg,

iii) antacid granules (a) CaCO₃ about 350-900 mg, (b) Mg(OH)₂ about 100-250 mg, (c) mannitol about 70-330 mg, (d) sorbitol about 30-90 mg, (e) crospovidone about 0-50 mg, (f) silica about 0-10 mg, and (g) purified water, and

iv) excipients for formulation of the tablet (a) diluent agent about 200-600 mg, (b) disintegrating agent about 50-300 mg, (c) aspartame about 10-40 mg, (d) flavorings about 10-30 mg, (e) silica about 5-15 mg, (f) magnesium stearate about 5-30 mg.

Water is used as solvent and removed during the coating and the granulation processes.

The microparticulate tablets according to the present invention may show an acid neutralizing capacity higher than about 10 mEq/tablet and after administration to subjects a rapid initial rise in gastric pH. In carrying out the objectives of the present invention, it is believed that the acid neutralizing capacity should be at least between about 10 and 25 mEq/tablet or higher. The enteric coating of the PPI microgranules is believed to comply with the requirements of the USP for enteric coated particles. The release of the PPI in the buffer stage at about pH 6.8 is believed to be not less than about 80% released in about 30 minutes. While the tablet is preferably round with a diameter of less than about 20 mm, the tablets may be of any suitable alternative shape, such as oval-shaped.

While the microparticulate tablets according to the present invention should have a hardness of not less than about 15 N, it is believed that such tablets should have a hardness of between about 20-70 N, when measured with the test method of the European Pharmacopeia (2.9.8) or equivalent.

The present invention also refers to the use of a tablet as described above for the manufacture of a medicament for the treatment of gastrointestinal disorders.

The tablet is administered one to several times a day, preferably once or twice daily. The typical daily dose of the active substances varies and will depend on various factors such as the individual requirements of the subject's and disease. In general, each tablet will comprise about 10-80 mg or more of a PPI, about 5-50 mg or more of a cholinergic agonist and about 200-1500 mg or more of an antacid agent. Preferably, each tablet will comprise about 10-40 mg of a PPI, about 5-25 mg of a cholinergic agonist and about 750-1000 mg of an antacid agents.

The present invention also provides methods for treating a subject in need of therapy for gastrointestinal disorders and/or preventing a gastrointestinal disorder in a subject. One embodiment of the method is as follows:

A method for treating or preventing gastrointestinal disorders comprising the steps of (a) combining a combination or single dosage form comprising a proton pump inhibitor in one distinct layer and an aluminum, magnesium or calcium antacid salt in another distinct layer into a chewable or rapidly dispersible dosage form comprising at least two layers, and (b) providing said chewable or rapidly dispersible dosage form to a patient in need of therapy for gastrointestinal disorders.

### Buccal Tablets

The bioavailability of a proton pump inhibitor after oral administration may be diminished due to premature activation upon exposure to the acidic conditions of the stomach and by degradation via hepatic fist pass metabolism. Transmucosal delivery of proton pump inhibitors provides an alternative route of administration that avoids these gastric and hepatic degradative processes, thereby rapidly increasing plasma levels of these drugs. The present invention provides pharmaceutical compositions of proton pump inhibitors for transmucosal delivery and cholinergic agonist for oral and transmucosal delivery.

A “unidirectional film” is designed to allow for substantially one-sided delivery of a proton pump inhibitor across the oral mucosa. It substantially prevents delivery of a proton pump inhibitor across the film.

The term “water impermeable layer” as used in this invention includes any film, coating or other substrate that substantially prevents delivery of PPI across such layer.

A term “multiple compressed tablet”, as used herein, is a tablet prepared by subjecting the fill material to more than a single compression.

Examples of “bioadhesive polymers”, as used herein, include, for example, alkyl celluloses, polysaccharides, polypeptides, synthetic polymers and mixtures thereof.

“Synthetic polymers” that may be used as bioadhesive polymers include, for example, vinyl and acrylic derivatives of carbomer, polycarbophil, polyethylene glycol, polyethylene oxide, polymethacrylates, polyvinyl alcohol, polyvinylpyrrolidone, and the like.

“Alkyl celluloses” that may be used as bioadhesive polymers include, hydroxyethyl cellulose, hydroxypropyl cellulose, hydroxypropylmethyl cellulose, methyl cellulose, sodium carboxymethyl cellulose, calcium carboxymethyl cellulose, and the like.

“Polysaccharides” that may be used as bioadhesive polymers include, for example, acacia, agar, alginic acid and salts of alginic acid, carageenan, dextran, guar gum, karaya gum, pectin, tragacanth, xanthan gum, and the like.

“Polypeptides” that may be uses as bioadhesive polymers include, for example, casein, gelatin, protamine sulfate, and the like.

Examples of “permeation enhancers” suitable for use include medium chain triglycerides; bile salts; anionic surfactants such as docusate sodium and sodium lauryl sulfate; cationic surfactants such as benzalkonium chloride, benzethonium chloride, and cetrimide; non-ionic surfactants such as glyceryl monooleate, polyoxyethylene sorbitan fatty acid esters, polyvinyl alcohol, and sorbitan esters; alcohol(s); isopropyl myristate; oleic acid; and the like.

Examples of “solubility enhancers” suitable for use include buffers, cosolvents, surfactants, and complexants such as polyamidoamine starburst dendrimers and cyclodextrins.

“Rapidly dispersing agents” suitable for use herein include, for example, wicking agents (agents that transport moisture into the interior of a dosage form so that the dosage form can dissolve from the inside as well as from the outside), non-effervescent disintegrants, and effervescent disintegrants.

The term “wicking agents” as used herein includes various non-effervescent disintegration agents such as microcrystalline cellulose; croscarmellose sodium; crosslinked polyvinylpyrrolidone; starches such as corn and potato starches, and modified starches; alginates; gums such as agar, arabic, guar, locust bean, karaya, pectin, and tragacanth, Carbopol®, hydroxyalkyl cellulose, hydroxypropylmethyl cellulose and the like. Wicking agents also include effervescent disintegration agents including compounds that evolve gas. The effervescent agents typically evolve gas by means of chemical reactions that occur upon exposure of the effervescent disintegration agent to saliva. The gas generating reaction is usually the result of a reaction between a soluble acid source and an alkaline metal carbonate or carbonate source that generates carbon dioxide gas upon contact with the water in saliva. The acid sources that may be used in the effervescent agent are any that are safe for human consumption, for example, food acids, and hydrite antacids such as citric, tartaric, malic, fumaric, adipic, succinic acid, and the like. Carbonate sources include dry solid carbonate and bicarbonate salts such as sodium bicarbonate, sodium carbonate, potassium bicarbonate, potassium carbonate, calcium carbonate, magnesium carbonate, and the like.

The pharmaceutical composition of the present invention may be formed as a partitioned tablet, e.g., a bi-layered tablet or a multiple compressed tablet, that is made by compressing a dosage form including a proton pump inhibitor and a cholinergic agonist around a compressed antacid core, or a bi-layer unidirectional film, patch or tablet. Other oral solid dosage forms, such as single compressed tablets or molded tablets may also be used.

In use, the pharmaceutical composition may be applied to the intraoral mucosa, e.g., the buccal sublingual, gingival mucosa, or the palate, for application and absorption across the palate, buccal, sublingual, or gingival mucosa. In one embodiment, the coating or layer of non-enteric-coated proton pump inhibitor and cholinergic agonist are thought to disperse, and the proton pump inhibitor and cholinergic agonist are believed to then be absorbed into the bloodstream or the cholinergic acid swallowed and absorbed from the small intestine or gut. In other embodiments, the inner layer of the bi-layer unidirectional film or tablet contain a proton pump inhibitor and cholinergic agonist, which are believed to be absorbed across the intraoral mucosa and into the bloodstream. The proton pump inhibitor then suppresses acid production at the gastric proton pumps. The cholinergic agonist activated the parietal cells to induce rapid onset of PPI action without a food effect. In a further embodiment, a resultant core containing an antacid or layer containing an antacid is then chewed or swallowed to provide immediate heartburn relief.

In one embodiment of the invention, an antacid is contained in a core surrounded by an outer layer containing a PPI and a cholinergic agonist or individual layers containing a PPI and a cholinergic agonist, respectively. When individual layers are utilized, it is believed to be preferable to position the PPI layer directly over the antacid core and the cholinergic layer on top of the PPI layer.

The outer layer or layers around the antacid core is designed to deliver a therapeutically effective amount of a PPI and a cholinergic agonist by absorption through the oral mucosa. The remaining antacid core is then left intact until chewed or swallowed.

The amounts of PPI and cholinergic agonist included in the formulation may be any amount that is therapeutically effective, i.e., an effective gastric acid suppressing amount and a parietal cell activation amount, respectively. For example, the amount of PPI included in these and other formulations of the present invention may be between about 1-1000 mg as discussed herein before. In some embodiments of the present invention, the amount of PPI in the formulation is between about 5-150 mg, about 10-80 mg, or about 10-40 mg. For veterinary applications, the amount of PPI in the formulation may be that amount sufficient to provide from about 1-10 mg or about 2-5 mg of PPI per kg of body weight. Thus, a formulation intended for administration to a horse may contain, for example, from about 0.5 gm to about 10 gm, about 0.5 gm to about 5 gm, or from about 0.5 to about 3 gm. of PPI. With respect to the cholinergic agonist, the amount in the formulation is between about 0.1 mg-500 mg or more as also discussed above. In some embodiments of the present invention, the amount of cholinergic agonist in the formulation is between about 1-250 mg, about 2.5 mg-100 mg, about 5-50 mg, or about 10 mg-50 mg. Alternatively, the formulations may contain a cholinergic agonist in an amount of about 10 μg/kg to about 1000 μg/kg.

The PPI and cholinergic agonist may be in the form of a powder, micronized powder, microspheres, microgranules, granules or other solid forms.

Additionally, the rapidly dispersing PPI and cholinergic layer or layers around the inner core containing an antacid may contain one or more of the following: a rapidly dispersing agent, a second pharmaceutical, an excipient, a flavorant, a stabilizer, a coloring agent a binder, a filler, a diluent or other component related to formulation, as previously discussed.

Depending on the particular formulation and application, the amount of antacid in the pharmaceutical composition will vary. In one embodiment, the amount of antacid incorporated into the core may range from about 1-80 mEq acid neutralizing capacity (ANC) or more. In another embodiment the amount of antacid present in the core may range from about 3-60 mEq ANC. In veterinary applications, the amount of antacid may range from about 1-1000 mEq ANC, about 1-500 mEq ANC, or about 1-100 mEq ANC.

In contrast to most commercial formulations of PPIs that use an antacid or buffering agent to stabilize the PPI, one embodiment of the present invention contains a pharmaceutical composition that includes an antacid to provide relief from symptoms of gastrointestinal disorders, e.g., episodic heartburn, after an effective gastric acid suppressing amount of the PPI and a parietal cell activation amount of the cholinergic agonist have been administered. Although an antacid is typically used in the core, other pharmaceutically active agents may be substituted in its place, and the antacid is administered concomitantly or substantially together with such buccal composition. In one embodiment, the antacid core is formulated as a chewable tablet.

In another embodiment, the core containing an antacid and the layer or layers containing a PPI and a cholinergic agonist can be separated by a film or coating to provide a tactile sense that the PPI and cholinergic agonist have been dissolved and that the antacid is ready to be chewed or swallowed. The film/coating may comprise, for example, a sugar coat polymeric film, or any other tablet coating known in the art.

In addition to the above, the core containing an antacid or layer containing an antacid may contain one or more of the following: a rapidly dispersing agent, a second pharmaceutical, an excipient, a flavorant, a stabilizer, a coloring agent, a binder, a filler, a diluent or other component related to formulation.

In another embodiment of the invention, a bi-layer unidirectional buccal film may be comprised of a unidirectional outer layer and a bioadhesive inner layer that contains a PPI and a cholinergic agonist.

An outer layer may be made of a pharmaceutically accepted polymeric material which is water impermeable and does not swell in contact with moisture, such as polyethylene, polyurethane, Mylar® and the like.

The outer layer may also contain an absorbable gelatin film (Gelfilm®, Pharmacia Upjohn) as a flexible bioerodible backer layer.

Additionally, the outer layer may be coated with a waxy material to form a thin film. The waxy material may be used to prevent the PPI and cholinergic agonist from being released into the oral cavity, which results in the unidirectional release of the drug into the oral mucosa. Pharmaceutical grade wax such as Carnauba wax, Bees wax, Shea Butter, Candelilla, Glyceryl Behenate, and Carnauba derivatives may be used to impart this water impermeability in the outer layer. In one embodiment, a low melting wax may be chosen to avoid high temperature processing conditions, since most PPI's are thermally unstable. In another embodiment, the waxy material may be Carnauba wax.

Additionally, the outer layer may contain one or more of the following: an excipient, a flavorant, a stabilizer, a coloring agent, or other excipient related to formulation, as discussed above.

The inner layer of the bi-layer film includes at least one bioadhesive polymer, a PPI and a cholinergic agonist. The PPI is incorporated into the inner layer by either a pre-load or a post-load process. In one embodiment, permeation enhancers and/or solubility enhancers may be employed to assist the rate of transmucosal delivery. The solubility of a PPI and a cholinergic agonist may possibly be improved, if necessary, by possible complexation with cyclodextrin (alpha-, beta-, gamma-, or substituted cyclodextrin). Complexation may be possible either as a discrete step prior to the formulation or during the drug loading step.

The amount of PPI and cholinergic agonist included in the formulation may be any amount that is therapeutically effective, as discussed above.

The PPI and cholinergic agonist may be in the form of a powder, micronized powder, microspheres, microgranules, granules or other solid forms, as previously indicated herein.

Additionally, the inner layer may contain one or more of the following: a rapidly dispersing agent, a bioadhesive, a second pharmaceutical, an excipient, a flavorant, a stabilizer, a coloring agent, or other component related to formulation.

In a further embodiment of the invention, the bi-layer unidirectional buccal tablet contains a proton pump inhibitor and a cholinergic agonist in the inner layer and an outer layer comprising a waxy material which prevents the PPI and cholinergic agonist from being released into the oral cavity, resulting in the unidirectional release of the PPI and the cholinergic agonist into the oral mucosa.

The waxy material present in the outer layer of the bi-layer unidirectional tablet may be a pharmaceutical grade wax. Examples of pharmaceutical grade waxes suitable for the present invention include Carnauba wax, Bees wax, Shea Butter, Candelilla, Glyceryl Behenate, and Carnauba derivatives. In one embodiment, the waxy material may be glyceryl behenate (Compitrol 888, Gattefosse).

In a further embodiment, the waxy layer may aid in the compressibility of the outer layer in addition to providing water impermeability. The waxy layer may protect the PPI from the slightly acidic environment of the mouth, thereby possibly eliminating the need for an alkaline component in the formulation of the inner layer.

Additionally, the outer layer may contain one or more of the following: an excipient, a flavorant, a stabilizer, a coloring agent, a binder, a filler, a diluent or other component related to formulation, as discussed above.

The inner layer may include at least one bioadhesive polymer, a PPI and a cholinergic agonist. The amount of PPI included in the formulation may be any amount that is therapeutically effective, as discussed before.

Again, the PPI and cholinergic agonist may be in the form of a powder, micronized powder, microspheres, microgranules, granules, or other solid forms.

In one embodiment of the invention, the inner layer also includes an antacid. The antacid may protect the PPI from degradation in the acidic environment of saliva or maintain product shelf-life of the pharmaceutical composition. Thus, both the amount of antacid and the antacid itself will be determined from the objective of its use. For example, less antacid may be necessary if the purpose is to maintain shelf life than if the purpose is to maintain stability of the PPI in saliva.

In another embodiment, magnesium carbonate is used. Magnesium carbonate may act as both an antacid and a binder. For pharmaceutical compositions applied directly to the buccal mucosa, it may be desirable to use a lesser amount of antacid, e.g., less than about 1 mEq ANC, less than about 0.5 mEq ANC, or less than about 0.1 mEq ANC, to keep the size of the dosage form manageable with respect to mucosal adhesiveness and mobility.

In another embodiment, hydroxypropyl cellulose (HPC) or hydroxylpropyl methylcellulose (HPMC) is used as a bioadhesive component. Depending upon which HPC or HPMC is selected, the disintegration time can be adjusted, which may increase or decrease the time available for delivery by keeping the tablet from collapsing for a selected period of time.

In a further embodiment, the bitter taste often associated with a PPI such as omeprazole, may be masked by the addition of a flavorant or taste masking agent. For example, direct compression grade xylitol (Xylitab 100 by Roquet) may impart a pleasing taste and mouth feel for the application duration.

In another embodiment, the inner layer contains a lubricant, for example, stearic acid or magnesium stearate.

In yet another embodiment of the invention, the antacid may be provided as a layer adjacent to the PPI and cholinergic agonist layer, e.g., as with a film, or simply administered concomitantly or substantially together with the buccal medicament.

Additionally, the inner layer may contain one or more of the following: a rapidly dispersing agent such as a wicking agent, a bioadhesive, a second pharmaceutical, an excipient, a flavorant, a stabilizer, a coloring agent, a binder, a filler, a diluent or other component related to formulation, as discussed above.

The pharmaceutical compositions of the present invention may be formulated as partitioned tablets, films, or any other solid, semi-solid, gel, or paste oral dosage form known in the art. For example, the pharmaceutical composition can be a molded or compressed tablet which may include one or more binder, diluent, adhesive, wicking agent, absorption enhancer such as a permeability enhancer and/or a solubility enhancer, lubricant, flavorant, or coloring agent, as previously described herein.

In one embodiment the pharmaceutical composition is formed by selecting a PPI dosage form and a cholinergic agonist dosage form and compressing the PPI and cholinergic agonist dosages around the core containing an antacid. In another embodiment, the PPI and cholinergic agonist are in the dosage form of a micronized powder.

In a further embodiment, a layered tablet or film is formed by configuring the layered tablet or film to have an inner layer to be in contact with the oral mucosal surface and an outer layer surface to allow for substantially one-sided delivery of the PPI and the cholinergic agonist across the oral mucosa.

In other embodiments of the present invention, the pharmaceutical composition may possibly be prepared by techniques widely known in the art such as wet or dry granulation, direct compression, or molding.

In contrast to various PPI formulations currently in commercial use, the pharmaceutical compositions embodied in the present invention provide the option of on-demand usage by the subject, because the pharmaceutical compositions of this invention may be taken on an empty stomach, without a meal or after a meal, or whenever needed (prn) to induce rapid onset of PPI action. In addition, the pharmaceutical compositions of the present invention may contain an antacid. For example, the pharmaceutical composition can be placed on an oral mucosal surface such as the sublingual mucosa, buccal mucosa, gingiva, or palate where the PPI and cholinergic agonist are absorbed.

In another embodiment, the PPI may be absorbed through the oral mucosa into the bloodstream. In further embodiments, an effective gastric acid suppressing amount of the PPI and a parietal cell activation amount of the cholinergic agonist are possibly absorbed within about 60 minutes, within about 30 minutes, within about 15 minutes or within about 10 minutes after placing it on the oral mucosa.

In another embodiment, the PPI and cholinergic agonist are absorbed leaving a core containing an antacid or a layer containing an antacid each of which may provide GI disorder relief, such as heartburn relief, when the patient chews or swallows the core containing the antacid or the layer containing the antacid.

In various embodiments, the pharmaceutical composition may be used for the treatment or prevention of gastrointestinal disorders. Treatment of these conditions and/or symptoms of these conditions may be accomplished by administering to a subject a safe and effective amount of the pharmaceutical composition according to the present invention.

In another embodiment according to the present invention, PPI buccal adhesive tablets to attach in the buccal cavity, such as the cheek, are disclosed.

In one example of a buccal adhesive tablet in accordance with the present invention is a buccal adhesive tablet that comprises a proton pump inhibitor, a cholinergic agonist, a bioadhesive additive and a saliva stabilizer to stabilize or protect the PPI from saliva. In this embodiment, the buccal adhesive tablet comprises sodium alginate and HPMC as the bioadhesive additive, magnesium oxide as the salvia stabilizer and croscarmellose as the releasing agent for the PPI and the cholinergic agonist.

The buccal adhesive tablets discussed herein may be prepared by compressing all of the ingredients together using, for example, a Erweka tablet machine (Frankfurt, Germany).

Qualitatively speaking, the buccal adhesive formulations may comprise

| | | |
|---|---|---|
| (a) | PPI | about 10 mg-100 mg or more |
| (b) | cholinergic agonist | about 5 mg-100 mg or more |
| (c) | adhesive | about 20 mg-100 mg or more |
| (d) | stabilizer | about 25 mg-100 mg or more and/or |
| (e) | releasing agent | about 5 mg-25 mg or more |

Examples of buccal adhesive tablets that are believed to be suitable comprise the following, plus other pharmaceutically acceptable excipients, such as flavorings, taste masking agents and the like as described above:

| | | | | |
|---|---|---|---|---|
| (a) | PPI - omeprazole | 10 mg | 20 mg | 40 mg |
| (b) | cholinergic agonist - bethanecol | 25 mg | 25 mg | 25 mg |
| (c) | sodium alginate | 28 mg | 28 mg | 28 mg |
| (d) | HPMC | 2 mg | 2 mg | 2 mg |
| (e) | magnesium oxide | 50 mg | 50 mg | 50 mg |
| (f) | croscarmellose | 10 mg | 10 mg | 10 mg |
| (a) | PPI - omeprazole | 10 mg | 20 mg | 40 mg |
| (b) | cholinergic agonist - bethanecol | 25 mg | 25 mg | 25 mg |
| (c) | sodium alginate | 25 mg | 25 mg | 25 mg |
| (d) | HPMC | 5 mg | 5 mg | 5 mg |
| (e) | magnesium oxide | 60 mg | 60 mg | 60 mg |
| (f) | croscarmellose | 20 mg | 20 mg | 20 mg |

### EXAMPLE 1

Immediate release tablets formed containing PPI and anticholinergics can be prepared by a variety of techniques know to those skilled in the art. In one such class of formulation, tablets formed by direct compression are formulated containing a PPI and an anti-cholinergic agent can be formulated by direct compression in tablets for coincident release. Tablets can be prepared from the ingredients below and milled granulations using a Manesty Betapress (Thomas Engineering, Inc., Hoffman Estates, Ill.). A typical tablet can be formulated using the components in Table 1.

TABLE 1

| | Quantity per | Quantity per |
|---|---|---|
| Ingredients | tablet (mg) | 10,000 tablets (gm) |
| omeprazole | 40 | 400 |
| bethanecol | 50 | 500 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of omeprazole in the form of salts, is the preferred PPI and would contain one half the quantity of racemic omeprazole in a typical tablet.

### EXAMPLE 2

Another formulation containing PPI and anticholinergics in an immediate release tablet can be formed using the techniques outlined in Example 1. Tablets formed by direct compression using a esomeprazole are formulated using the ratios in Table 2 below. Tablets can be prepared from the ingredients below and milled granulations using a Manesty Betapress (Thomas Engineering, Inc., Hoffman Estates, Ill.).

TABLE 2

| | Quantity per | Quantity per |
|---|---|---|
| Ingredients | tablet (mg) | 10,000 tablets (gm) |
| esomeprazole | 40 | 400 |
| bethanecol | 50 | 500 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of esomeprazole in the form of salts, is the preferred PPI and would contain one half the quantity of racemic esomeprazole in a typical tablet.

### EXAMPLE 3

Another formulation containing PPI and anticholinergics in an immediate release tablet can be formed using the techniques outlined in Example 1. Tablets formed by direct compression using rabeprazole are formulated using the ratios in Table 3 below. Tablets can be prepared from the ingredients below and milled granulations using a Manesty Betapress (Thomas Engineering, Inc., Hoffman Estates, Ill.).

TABLE 3

| | Quantity per | Quantity per |
|---|---|---|
| Ingredients | tablet (mg) | 10,000 tablets (gm) |
| rabeprazole | 20 | 200 |
| bethanecol | 50 | 500 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of rabeprazole in the form of salts, is the preferred PPI and would contain one half the quantity of racemic rabeprazole in a typical tablet.

### EXAMPLE 4

Another formulation containing PPI and anticholinergics in an immediate release tablet can be formed using the techniques outlined in Example 1. Tablets formed by direct compression using pantoprazole are formulated using the ratios in Table 4 below. Tablets can be prepared from the ingredients below and milled granulations using a Manesty Betapress (Thomas Engineering, Inc., Hoffman Estates, Ill.).

TABLE 4

| | Quantity per | Quantity per |
|---|---|---|
| Ingredients | tablet (mg) | 10,000 tablets (gm) |
| pantoprazole | 40 | 400 |
| bethanecol | 50 | 500 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of pantoprazole in the form of salts, is the preferred PPI and would contain one half the quantity of racemic pantoprazole in a typical tablet.

### EXAMPLE 5

Another formulation containing PPI and anticholinergics in an immediate release tablet can be formed using the techniques outlined in Example 1. Tablets formed by direct compression using lansoprazole are formulated using the ratios in Table 5 below. Tablets can be prepared from the ingredients below and milled granulations using a Manesty Betapress (Thomas Engineering, Inc., Hoffman Estates, Ill.).

TABLE 5

| | Quantity | Quantity per |
|---|---|---|
| | per tablet | 10,000 tablets |
| Ingredients | (mg) | (gm) |
| lansoprazole | 15 | 150 |
| bethanecol | 50 | 500 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of lansoprazole in the form of salts, is the preferred PPI and would contain one half the quantity of racemic lansoprazole in a typical tablet.

### EXAMPLE 6

Immediate release tablets formed containing PPI and anticholinergics can be prepared by a variety of techniques know to those skilled in the art. In one such class of formulation, tablets formed by direct compression are formulated containing a PPI and an anti-cholinergic agent can be formulated by direct compression in tablets for coincident release. A typical tablet can be formulated using the components in Table 6. Tablets can be prepared from the ingredients below and milled granulations using a Manesty Betapress (Thomas Engineering, Inc., Hoffman Estates, Ill.).

TABLE 6

| | Quantity | Quantity per |
|---|---|---|
| | per tablet | 10,000 tablets |
| Ingredients | (mg) | (gm) |
| omeprazole | 40 | 400 |
| pilocarpine | 7.5 | 75 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of omeprazole in the form of salts, is the preferred PPI and would contain one half the quantity of racemic omeprazole in a typical tablet.

### EXAMPLE 7

Another formulation containing PPI and anticholinergics in an immediate release tablet can be formed using the techniques outlined in Example 1. Tablets formed by direct compression using esomeprazole and pilocarpine are formulated using the ratios in Table 7 below. Tablets can be prepared from the ingredients below and milled granulations using a Manesty Betapress (Thomas Engineering, Inc., Hoffman Estates, Ill.).

TABLE 7

| | Quantity | Quantity per |
|---|---|---|
| | per tablet | 10,000 tablets |
| Ingredients | (mg) | (gm) |
| esomeprazole | 40 | 400 |
| pilocarpine | 7.5 | 75 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of esomeprazole in the form of salts, is the preferred PPI and would contain one half the quantity of racemic esomeprazole in a typical tablet.

### EXAMPLE 8

Another formulation containing PPI and anticholinergics in an immediate release tablet can be formed using the techniques outlined in Example 1. Tablets formed by direct compression using rabeprazole and pilocarpine are formulated using the ratios in Table 8 below. Tablets can be prepared from the ingredients below and milled granulations using a Manesty Betapress (Thomas Engineering, Inc., Hoffman Estates, Ill.).

TABLE 8

| | Quantity | Quantity per |
|---|---|---|
| | per tablet | 10,000 tablets |
| Ingredients | (mg) | (gm) |
| rabeprazole | 20 | 200 |
| pilocarpine | 7.5 | 75 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of rabeprazole in the form of salts, is the preferred PPI and would contain one half the quantity of racemic rabeprazole in a typical tablet.

### EXAMPLE 9

Another formulation containing PPI and anticholinergics in an immediate release tablet can be formed using the techniques outlined in Example 1. Tablets formed by direct compression using pantoprazole and pilocarpine are formulated using the ratios in Table 9 below. Tablets can be prepared from the ingredients below and milled granulations using a Manesty Betapress (Thomas Engineering, Inc., Hoffman Estates, Ill.).

TABLE 9

| | Quantity | Quantity per |
|---|---|---|
| | per tablet | 10,000 tablets |
| Ingredients | (mg) | (gm) |
| pantoprazole | 40 | 400 |
| pilocarpine | 7.5 | 75 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of pantoprazole in the form of salts, is the preferred PPI and would contain one half the quantity of racemic pantoprazole in a typical tablet.

### EXAMPLE 10

Another formulation containing PPI and anticholinergics in an immediate release tablet can be formed using the techniques outlined in Example 1. Tablets formed by direct compression using lansoprazole and pilocarpine are formulated using the ratios in Table 10 below. Tablets can be prepared from the ingredients below and milled granulations using a Manesty Betapress (Thomas Engineering, Inc., Hoffman Estates, Ill.).

TABLE 10

| | Quantity | Quantity per |
|---|---|---|
| | per tablet | 10,000 tablets |
| Ingredients | (mg) | (gm) |
| lansoprazole | 15 | 150 |
| pilocarpine | 7.5 | 75 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

It should be noted that the (s)-isomer of lansoprazole in the form of salts, is the preferred PPI and would contain one half the quantity of racemic lansoprazole in a typical tablet.

### EXAMPLE 11

Tablets may also be formulated using a variety of additional excipients and combinations of sugars which can include buffers aimed at maintaining the stability of the PPI which tend to be acid instable. One such formulation containing PPI, anticholilnergic and a phosphate buffer such as Na₂HPO₄ is described in Table 11. Tablets can be prepared from the ingredients below and milled granulations using a Manesty Betapress (Thomas Engineering, Inc., Hoffman Estates, Ill.).

TABLE 11

| | Quantity | Quantity per |
|---|---|---|
| | per tablet | 10,000 tablets |
| Ingredients | (mg) | (gm) |
| lansoprazole | 15 | 150 |
| bethanecol | 50 | 500 |
| Na₂HPO₄ | 30 | 300 |
| Ludipress (BASF, Lactose, | 153.75 | 1537.5 |
| povidone, crospovidone) | | |
| Magnesium stearate | 1.25 | 12.5 |

It is understood by those skilled in the art that additional examples can be formulated using the various combination of PPI's and anti-cholinergics and buffers outlined in examples 1 through 11 in addition to other PPI's and anti-cholinergics not mentioned in these examples.

### EXAMPLE 12

Tablets such as those described in examples 1 through 11 can be enteric coated using a variety of materials such as Eudragit (Rohm and Haas) or Kollicoat MAE 30 DP and 100 DP (BASF) methacrylic acid polymers and copolymers. These enteric coatings are designed to protect, retard or delay the release of the active PPI and/or anticholinergic.

A basic formulation for enteric coating of tablets using Eudragit L 30 D-55 and Kollicoat is shown in Table 12A and 12B.

TABLE 12A

| Ingredient | Quantity (grams) |
|---|---|
| Eudragit L 30 D-55 | 1670 |
| Water | 3335 |
| Triethyl citrate | 50 |
| Talc | 830 |
| Titanium dioxide/pigments | 500 |
| Sodium carboxymethylcellulose | 35 |
| Polyethylene glycol 6000 | 80 |
| Water | 3500 |

EUDRAGIT® L 30 D-55 is an aqueous dispersion of an anionic polymethacrylate. It is insoluble in acid media, but dissolves above pH 5.5.

The preparation of the enteric coating often requires the addition of a plasticizer (10%) such as triethyl citrate and 25 to 100% talc. The amount of polymer used for enteric coatings can be varied based on the polymer used. A typical amount used ranges from 1 to 8 mg/cm² per tablet surface. This amount can be increased or decreased depending on the release properties needed for the tablet being coated. Thicker coatings may increase the disintegration time in the intestinal fluid. Pigments such as TiO₂ or other color lakes maybe added to increase the permeability of the coatings.

TABLE 12B

| | Coating | Coating |
|---|---|---|
| | solution I. | solution II. |
| Ingredient | Weight (grams) | Weight (grams) |
| Kollicoat MAE 30DP | 50 | — |
| Kollicoat MAE 100DP | — | 15 |
| Triethyl citrate | 2.25 | 2.25 |
| Water | 32.25 | 67.25 |
| Sicovit Red 30 | 0.5 | 0.5 |
| Pigment | | |
| (BASF) | | |
| Titanium Dioxide | 0.5 | 0.5 |
| Talc | 4.0 | 4.0 |
| Water | 10.5 | 10.5 |

The Eudragit or Kollicoat solutions are spray coated onto the material to be enteric coated using conventional equipment such as coating pans, perforated pans, or fluid bed coaters. Coating pans are generally used for tablets such as an Accela Cota 24 (Manesty) equipped with a Schlick 930/7-1-S35 spray gun. Glatt fluid bed coaters can be used for coating small particles or microspheres.

### EXAMPLE 13

Capsules containing a PPI and an anticholinergic agent are prepared using the excipients that allow the formulation of free flowing powders to be contained within the capsule dosage form. Maltodextrin carbohydrates (M500, M510,_, Grain Processing Corporation), microcrystalline cellulose are effective carriers and bulking agents for filling hard gelatin or hydroxypropyl methylcellulose and the like. The capsule can be formulated containing 40 mg of pantoprazole, 50 mg bethanecol and a bulking agent such as 310 mg maltodextrin. Other PPI's or cholinergics can be substituted in such a capsule formulation by those skilled in the art.

### EXAMPLE 14

Enteric coated capsules containing a PPI and an anticholinergic agent are prepared using the excipients that allow the formulation of free flowing powders to be contained within the capsule dosage form. Coating formulations such as those described in Example 12 can be used to delay the release of the PPI and the anticholinergic in the gastrointestinal tract. Maltodextrin carbohydrates (M500, M510, Grain Processing Corporation), or microcrystalline cellulose are effective carriers and bulking agents for filling hard gelatin or hydroxypropyl methylcellulose and the like. The capsule can be formulated containing 40 mg of omeprazole, 50 mg bethanecol and a bulking agent such as 310 mg maltodextrin. Other PPIs or anticholinergics can be substituted in such a capsule formulation by those skilled in the art. Alternatively, the PPI and cholinergic agonist can each be formed into enteric coated beads and capsule filled so that the PPI is released from its beads at a pH of about 5 or greater and preferably at a pH of between about 6 and about 7, and the cholinergic agonist is released from its beads at between about 10 minutes to about 60 minutes, and preferably between about 15 minutes and about 30 minutes, after PPI release.

### EXAMPLE 15

The combination of a PPI and an anticholinergic can also be formulated in a syrup. Such a syrup vehicle for the combination of 40 mg of omeprazole, 7.5 mg pilocarpine can be dissolved in 10 mL of aqueous solution. A solution of suitably flavored volatile oil, (vanillin for example) is diluted in glycerin. Two mL of vanillin oil is diluted with 6 mL of ethyl alcohol which is then added to 500 mL of glycerin. After complete dissolution the vanillin, the solution is filled to 1000 mL final volume. A solution or suspension of 40 mg of omeprazole and 7.5 mg pilocarpine is combined with 10 mL of syrup in order to form a 20 mL liquid dosage form of the PPI and anticholinergic. Addition of maltodextrins cane be used to vary osmolarity, sweetness and viscosity. Other PPI's or anticholinergics can be substituted in such a liquid formulation by those skilled in the art.

### EXAMPLE 16

Table 13 shows a representative formulation comprised of a 3 layer tablet containing a PPI such as omeprazole in its core. The tablet is then coated in the second layer with an acid protective enteric layer and finally a third layer containing the anticholinergic such as pilocarpine for immediate release.

TABLE 13

| | Quantity | Quantity per |
|---|---|---|
| | per tablet | 10,000 tablets |
| Ingredients | (mg) | (gm) |
| omeprazole | 40 | 400 |
| Compressible Sugar, NF | 54 | 540 |
| (Di-Pac ®, NU-TAB ® | | |
| 4001, SugarTAB ®) | | |
| Sterotex NF lubricant | 4.0 | 40 |
| SYLOID 244FP silica | 2.0 | 20 |
| dispersant | | |

The second layer enteric coating is formulated using a formula outlined in Table 14. The second layer should be applied in the range of 4 to 8 mg/cm² per tablet surface.

TABLE 14

| Ingredient | Quantity (grams) |
|---|---|
| Eudragit L 30 D-55 | 1670 |
| Water | 3335 |
| Triethyl citrate | 50 |
| Talc | 830 |
| Titanium dioxide/pigments | 500 |
| Sodium carboxymethylcellulose | 35 |
| Polyethylene glycol 6000 | 80 |
| Water | 3500 |

The Eudragit solution is spray coated onto the material to be enteric coated using conventional equipment such as coating pans, perforated pans, or fluid bed coaters. Coating pans are generally used for tablets such as an Accela Cota 24 (Manesty) equipped with a Schlick 930/7-1-S35 spray gun. Glatt fluid bed coaters can be used for coating small particles or microspheres. A final layer is then spray coated at 1 mg/cm² per tablet surface containing the anticholinergic agent by dissolving pilocarpine within the coating solution to ensure rapid release of the pilocarpine using the formula shown in Table 15.

TABLE 15

| Ingredient | Quantity (grams) |
|---|---|
| Eudragit L 30 D-55 | 1595 |
| Water | 3335 |
| Pilocarpine | 150 |
| Triethyl citrate | 50 |
| Talc | 755 |
| Titanium dioxide/pigments | 500 |
| Sodium carboxymethylcellulose | 35 |
| Polyethylene glycol 6000 | 80 |
| Water | 3500 |

Any PPI can be incorporated into the core tablet and any anticholinergic can be incorporated into the third coating for immediate release. The anticholinergic can also be incorporated in to the core with the PPI in either a second protected layer or the outer immediate release layer, as depicted in Example 20.

### EXAMPLE 17

Another useful formulation containing a combination of a PPI and an anticholinergic is formulated by enterically coating microparticulate forms of the PPI and/or the anticholinergic and then incorporating the formulation into a tablet, a chewable table, a capsule or even an liquid suspension.

Particles of rabeprazole in the range of 300 to 1.200 microns are prepared by crystallization and micronization. Table 16 shows a Eudragit L 30 D-55 formulation designed to coat 1000 grams of rabeprazole.

TABLE 16

| Ingredient | Quantity (grams) |
|---|---|
| Eudragit L 30 D-55 | 265 |
| Triethyl citrate | 8 |
| Talc | 40 |
| Antifoam emulsion | 1 |
| Water | 186 |

The crystals, pellets or granules of rabeprazole is coated in a fluid bed coater with granulator (Uni-Glatt).

The coated microparticles containing 1000 grams of rabeprazole are combined with 2500 grams of bethanecol. The formulation is can then be formulated into oral tablet capsule or liquid oral formulation. One formulation of such a tablet is shown in Table 17.

TABLE 17

| | Quantity | Quantity |
|---|---|---|
| | per tablet | per |
| Ingredients | (mg) | 7100 (gm) |
| Eudragit Coated | 20 active rabeprazole | 600 |
| rabeprazole | (67 mg of | |
| | Eudragit coated | |
| | rabeprazole) | |
| bethanecol | 50 | 500 |
| Compressible Sugar, NF | 54 | 5400 |
| (Di-Pac ®, NU-TAB ® | | |
| 4001, SugarTAB ®) | | |
| Sterotex NF lubricant | 4.0 | 400 |
| SYLOID 244FP silica | 2.0 | 200 |
| dispersant | | |

It should be noted that the (s)-isomer of rabeprazole in the form of salts, is the preferred PPI and would contain one half the quantity of racemic rabeprazole in a typical tablet.

### EXAMPLE 18

The Eudragit coated PPI described in example 17 and an anticholinergic can also be incorporated into a chewable antacid tablet form. Such a formulation is described in Table 18.

TABLE 18

| | | Per |
|---|---|---|
| Ingredient | Per tablet (mg) | 10,000 (gm) |
| Magnesium Trisilicate | 500 | 5000 |
| Aluminum hydroxide | 250 | 2500 |
| Mannitol | 300 | 3000 |
| Splenda sweetener | 1 | 20 |
| Magnesium stearate | 10 | 100 |
| Corn starch | 10 | 100 |
| Eudragit coated | 67 (contains | 670 |
| rabeprazole | 20 mg active) | |

### EXAMPLE 19

### Animal Study

### PPI+Cholinergic Stimulation

Rats are fasted overnight.

Rats are anesthetized and small laparotomy and gastrotomy are performed.

Tygon tubing placed through gastrotomy and into proximal duodenum; pylorus ligated

Tubing is placed through a small gastrotomy into the most dependent portion of the stomach; esophagogastric junction is ligated carefully to avoid vagal damage and to prevent aspiration

Gastrotomy and abdomen is closed around tubing

Stomach is gently rinsed with about 0.9% NaCl at about pH 7.

Gastric contents are collected by gravity for about 30 min, at which time is rinsed with about 5 ml of NaCl and collected; 30-min contents is titrated with about 0.01 NaOH using radiometer automatic titrator to determine basal acid output (“BAO”)

Rats (3 per group) are randomized to 1 of 3 treatments:
A. Control—intraduodenal (ID) HCO₃;
B. Carbachol about 15 μg/kg IP+ID HCO₃; and
C. ID omeprazole (about 20 mg/kg) is dissolved in HCO₃+carbachol about 15 μg/kg IP

Gastric contents are collected by gravity for about 30 min, at which time stomachs are rinsed with about 5 ml of NaCl and collected; contents are titrated with 0.01 M NaOH using a Radiometer pH meter; about 30-min collection is repeated ×6.

The results are shown in FIG. 1, wherein carbachol rapidly stimulates acid secretion, which reaches a maximum level at about 90 min following administration and gradually diminishes thereafter. When carbachol and omeprazole are administered concomitantly, an increase in acid secretion is detected at about 30 min. By about 60 min, however, acid secretion in this group of animals is indistinguishable from basal acid output, indicating rapid onset of action of the PPI by the use of cholinergic stimulation.

While the present invention has been described in the context of numerous embodiments and example, it will be readily apparent to those skilled in the art that other modifications and variations can be made therein without departing from the spirit or scope of the present invention. Accordingly, it is not intended that the present invention be limited to the specifics of the foregoing description of the exemplary embodiments and example, but rather as being limited only by the scope of the invention as defined in the claims appended hereto.

### EXAMPLE 20

Example PPI— Cholinergic Targeted and Sequential Release Tablet

One embodiment of the present invention, a pharmaceutical composition is formed by forming a core containing “a solid or granular or crystalline form of the cholinergic such as carbachol, bethanechol or pilocarpine. This core can be a polymeric matrix formulated from the bethanechol or pilocarpine within acceptable enteric polymer coatings such as cellulose acetate phthalate, cellulose acetate trimellitate, hydroxypropylmethylcellulose phthalate, copolymers of methacrylic acid and ethyl acrylate (Eudragit), hydroxypropylmethylcellulose acetate succinate and polyvinyl acetate phthalate. The core is then coated with a fast dissolving coating containing the PPI. A typical fast dissolving polymeric enteric polymer is cellulose acetate phthalate. In this way the PPI will dissolved first, followed by the delayed release of the cholinergic core or core matrix. The fast dissolving coating containing the PPI can then be coated with a coating that release the PPI at a pH of at least about pH 5 and more preferably at between a pH of about 6 and about 7.

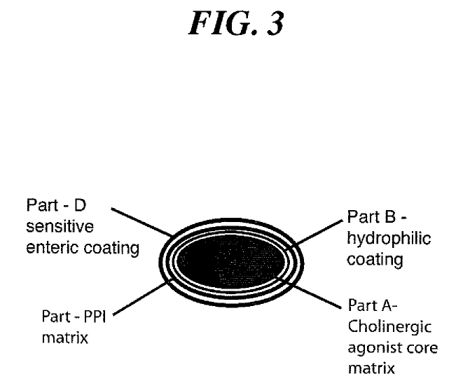

In another embodiment, a cholinergic agonist such as pilocarpine, carbachol, bethanecol chloride etc, is combined with a suitable filler such as lactose, sucrose, cellulose or similar binding substances and formed into a tablet core. (Part A). As depicted in FIG. 3, this core material is next coated with a hydrophilic polymer such as polyethylene glycol (PEG), polyvinylpyrrolidone (PVP), poloxamer etc., which will allow the dissolution of the cholinergic core over a 10 to 20 minute period. (Part B). It should be appreciated that Part B, the hydrophilic coating of the cholinergic agonist may or may not be necessary to control the dissolution kinetics of the cholinergic agonists. A proton pump inhibitor such as omeprazole, lansoprazole etc, is then formulated into a matrix coating which is deposited upon Part B, using a rapid dissolve filler matrix material composed of lactose, sucrose, cellulose or similar material (Part C). The PPI matrix (Part C.) is then finally coated with an enteric coating designed to release the PPI at a pH above 5.5, targeting the duodenum (Part D). Such pH sensitive enteric polymer coatings include cellulose acetate phthalate, cellulose acetate trimellitate, hydroxylpropylmethyl cellulose phthalate and the methacrylic copolymers. A particularly good pH sensitive methacrylic polymer coating is Eudragit (DeGussa, Düsseldorf, Germany) L100-55.

Carbachol, pilocarpine or bethanechol granules can be formulated in a manner that will accelerate the release of the cholinergic. Methods of preparing such granules include spray drying the pilocarpine or bethanechol from a suitable solvent into a non-solvent which will form high surface granules. These granules can be collected and dried and formulated into a suitable tablet.

Alternatively, high surface area nanoparticles of these cholinergics can be formed by homogenization. A suspension of the compound is formulated in a non-solvent and then exposed to high shear mixing using devices such as a microfluidizer, a mechanical high shear device or an ultrasonic homogenizer. These nanoparticles can then be incorporated into a suitable tablet.

Nanoparticles can also be formed by dissolving the cholinergic in an solvent and precipitating the compound in the presence of a non-solvent. Fast dissolving nanoparticles are optimally formed at less than 1000 nm in particle size diameter, and more preferably less than 500 nm in diameter and most preferably less than 100 nm in diameter.

1-168. (canceled)

169. An orally administrable pharmaceutical composition for a patient in need of proton pump inhibitor treatment to treat or prevent a gastric acid disorder, said pharmaceutical composition comprising
(a) an effective gastric acid suppressing amount of a proton pump inhibitor, and
(b) an effective parietal cell activation amount of a cholinergic agonist, wherein the proton pump inhibitor is released from said pharmaceutical composition at a pH of about 5 or higher following oral administration and the cholinergic agonist is released from said pharmaceutical composition within between about 10 minutes and 60 minutes after release of the proton pump inhibitor from said pharmaceutical composition.

170. An orally administrable pharmaceutical composition of claim 169, wherein the cholinergic agonist is released from said pharmaceutical composition within between about 15 minutes and 45 minutes after release of the proton pump inhibitor from said pharmaceutical composition.

171. An orally administrable pharmaceutical composition of claim 169, wherein the cholinergic agonist is released from said pharmaceutical composition within between about 20 minutes and 40 minutes after release of the proton pump inhibitor from said pharmaceutical composition.

172. An orally administrable pharmaceutical composition of claim 169, wherein the cholinergic agonist is released from said pharmaceutical composition within about 30 minutes after release of the proton pump inhibitor from said pharmaceutical composition.

173. An orally administrable pharmaceutical composition of claims 169-172, wherein said pharmaceutical composition further includes an antacid.

174. An orally administrable pharmaceutical composition of claims 169-173, wherein the proton pump inhibitor is selected from the group consisting of dontoprazole, esomeprazole, habeprazole, hydroxyomeprazole, lansoprazole, leminoprazole, pantoprazole, pariprazole, perprazole, (s-omeprazole magnesium) omeprazole, omneirazole, rabeprazole, ransoprazole, tenooprazole, TU-199 and mixtures thereof in neutral form, as well as the pharmaceutically acceptable salt, prodrug, derivative, R- or S-enantiomer, isomer, free base, anhydrate, hydrate, solvate, polymorph or combinations thereof.

175. An orally administrable pharmaceutical composition of claim 169-173, wherein the proton pump inhibitor is selected from the group consisting of omeprazole, lansoprazole, rabeprazole, pantoprazole and esomeprazole.

176. An orally administrable pharmaceutical composition of claims 169-173, wherein the proton pump inhibitor is selected from the group consisting of omeprazole, lansoprazole, and esomeprazole.

177. An orally administrable pharmaceutical composition of claims 169-173, wherein the proton pump inhibitor is omeprazole.

178. An orally administrable pharmaceutical composition of claims 169-173, wherein the proton pump inhibitor is lansoprazole.

179. An orally administrable pharmaceutical composition of claims 169-173, wherein the proton pump inhibitor is esomeprazole.

180. An orally administrable pharmaceutical composition of claims 169-179, wherein said cholinergic agonist is selected from the group consisting of methacholine, carbachol, bethanecol, arecholine, aceclidine, acetylcholine, pilocarpine, muscarine, acyclidine, oxotremorine, physostigmine, neostigmine, edrophonium, pyridostigmine, demecarium, ambenonium, cis-2-methyl-5-trimethylammoniummethyl-1,3-oxathiolane iodide (OXA-22), McN-A-343, CL-1017, RS-86 (2-ethyl-8-methyl-2,8-diazospiro-4,5-decan-1,3-dianhydrobromide), AF102B [±cis-2-methyl-spiro(1,3-oxathiolane-5,3′) quinuclidine], azaspirodecanes (2-methyl-1,3-dioxaazaspiro[4,5]-decanes), tetrahydroaminoacridine, HP 029, galanthamine, 9-Amino-1,2,3,4-tetrahydroaminoacridine (THA), linopirdine [DuP 996; 3,3-bis(4-pyrindinylmethyl)-1-phenylindolin-2-one], HP 749 [N-(n-propyl)-N-(4-pyridinyl)-1H-indol-1-amine], dexpanthenol; echothiophate iodide, isofluorophate, cis-dioxolane,(+)-, (4-hydroxy-2-butynyl)-1-trimethylammonium m-chlorocarbonilate chloride, talsaclidine, cevimeline, McN-A343, nicotine, S(−)-, isofluorophate, demecarium and echothiophate and mixtures thereof in neutral form, as well as the pharmaceutically acceptable salt, prodrug, R- or S-enantiomer, isomer, free base, anhydrate, hydrate, solvate, polymorph or combinations thereof.

181. An orally administrable pharmaceutical composition of claims 169-179, wherein said cholinergic agonist is selected from the group consisting of bethanecol, pilocarpine and carbachol.

182. An orally administrable pharmaceutical composition of claims 169-179, wherein said cholinergic agonist is bethanecol.

183. An orally administrable pharmaceutical composition of claims 169-179, wherein said cholinergic agonist is pilocarpine.

184. An orally administrable pharmaceutical composition of claims 169-179, wherein said cholinergic agonist is carbachol.

185. An orally administrable pharmaceutical composition of claims 169-184, wherein said pharmaceutical composition is packaged in a container with printed labeling advising that said pharmaceutical composition can be taken at anytime during the day or night without food or in a fasted state.

186. An orally administrable pharmaceutical composition of claims 169-185, wherein said pharmaceutical composition is packaged in a container with printed labeling advising that administration of said pharmaceutical composition results in an increase in at least one of C(max) and AUC(last) of the proton pump inhibitor at the time of parietal cell activation, as compared to administration of the proton pump inhibitor alone in a fasted state.

187-306. (canceled)
